(19) United States
(12) Patent Application Publication
(10) Pub. No.: US 2017/0276611 A1
(43) Pub. Date: Sep. 28, 2017

(54) CHALCOGENOPYRYLIUM DYES, COMPOSITIONS COMPRISING SAME, COMPOSITE NANOPARTICLES COMPRISING SAME, AND METHODS OF MAKING AND USING THE SAME

(71) Applicants: The Research Foundation for The State University of New York, Buffalo, NY (US); The University of Strathclyde, Glasgow (GB)

(72) Inventors: Michael DETTY, Rochester, NY (US); Matthew Allen BEDICS, Amherst, NY (US); Graham DUNCAN, Glasgow (GB); Karen Jane FAULDS, Glasgow (GB); Hayleign KEARNS, Glasgow (GB)

(21) Appl. No.: 15/528,607

(22) PCT Filed: Nov. 20, 2015
(86) PCT No.: PCT/US15/61791

§ 371 Date: May 22, 2017

Related U.S. Application Data
(60) Provisional application No. 62/082,554, filed on Nov. 20, 2014.

(51) Int. Cl. G01N 21/65; C09B 23/10; A61B 5/00; A61K 49/00; G01N 33/574; G01N 33/58; C09B 57/00; C09B 23/06

(52) U.S. Cl. CPC G01N 21/658; C09B 57/00; C09B 23/105; C09B 23/107; C09B 23/06; A61K 49/00; G01N 33/574; G01N 33/587; G01N 33/583; A61B 5/0075; G01N 2458/00

(57) ABSTRACT

The present disclosure provides chalcogenopyrylium compounds, composite nanostructures comprising the chalcogenopyrylium compounds, and methods of using the compounds and/or composite nanostructures. For example, composite nanostructures comprising the chalcogenopyrylium compounds are used in imaging applications. The present disclosure provides chalcogenopyrylium compounds having the following structure where each E is, at each occurrence in the compound, independently charged or neutral and is independently selected from S, Se, 0, or Te, wherein at least one E is S or Se; each R1 is, at each occurrence in the compound, independently selected from the group consisting of —H, Ci-s alkyl group, halo group, —CN, aryl group, and heteroaryl group and adjacent R1 groups can combine to form C5ss aryl groups, each R2 is, at each occurrence in the compound.

### CROSS-REFERENCE TO RELATED APPLICATIONS

This application claims priority to U.S. Patent Application No. 62/082,554, filed Nov. 20, 2014, the disclosure of which is hereby incorporated by reference.

### STATEMENT REGARDING FEDERALLY SPONSORED RESEARCH

This invention was made with government support under contract no. CHE-1151379 awarded by the National Science Foundation. The government has certain rights in the invention.

### FIELD OF THE DISCLOSURE

This disclosure relates generally to the field of chalcogenopyrylium compounds and surface-enhanced Raman spectroscopy and chalcogenopyrylium compositions used with surface-enhanced Raman spectroscopy.

### BACKGROUND

Surface-enhanced Raman scattering (SERS) has been utilized as a sensitive analytical tool in the study of biological systems. The combination of a metallic nanoparticle and an organic dye as a reporter molecule provide SERS nanotags that can be used to detect target molecules using laser Raman spectroscopy or SERS microscopy. This spectroscopic technique not only has high sensitivity (10⁻⁹ M-10⁻¹² M limits of detectability), but also the potential for multiplexing capabilities due to the unique vibrational structure of adsorbed molecules on the metallic nanoparticle. For most medical applications, the 785-nm laser has been used to excite SERS nanotags and, while systematic investigation of SERS reporter molecules has been limited, SERS reporters for this wavelength have been designed and utilized. Orders-of-magnitude higher sensitivities (10⁻¹²-10⁻¹⁴ M) can be achieved utilizing Raman reporters that are in resonance with the incident laser, thereby producing surface-enhanced resonance Raman scattering (SERRS) nanoprobes. [Note: SERS and SERRS are used interchangeably from this point forward.] The optical absorptance of human tissue is minimal in the 600-800-nm window and increases at longer wavelengths due to absorption by water. While the 785-nm laser operates within this window, the depth of penetration of infrared light increases at longer wavelengths due to decreased scattering, reaching a minimum near 1300 nm. The superior penetration depth of 1300-nm light vs. 800-nm light has been documented, but Raman scattering at 1300 nm is so weak that it may be impossible to use. Therefore, to exploit the advantages of the unique vibrational signatures produced by Raman scattering, surface enhancement of the signal must be used to operate at this longer wavelength of excitation. Lasers emitting in the 1500-nm to 1600-nm range are invisible to the human eye and exposure of the eye to these wavelengths is not damaging.

The region from 1000 nm to 1300 nm is of particular interest and is compatible with commercial laser excitation sources operating at 1064 and 1280 nm. SERS nanotags operating at 1064-nm have been described using crystal violet, rhodamine 6G, methylene blue, and 9-aminoacridine as reporter molecules. A direct comparison of the 1064-nm (Ti:sapphire) and 1280-nm (Cr:forsterite) lasers showed that the 1280-nm laser excitation gave reduced sample burning, limited photobleaching, reduced background fluorescence/autofluorescence, and greater penetration depth into biological tissues. The 1280-nm laser has been utilized in both optical coherence tomography and fluorescence microscopy, to take advantage of the superior penetration of 1280-nm light in turbid media such as tissue and blood. To date, there appear to be no SERS nanotags compatible with a 1280 nm excitation laser. Thus, there is great need to provide 1280-nm SERS nanotags in order to harness the significant benefits of operating at this wavelength of excitation. One possible application of SERS nanotags operating at this wavelength is human biomedical imaging of SERS nanotags targeted to specific sites such as tumors.

### BRIEF SUMMARY OF THE DISCLOSURE

The present disclosure provides chalcogenopyrylium compounds having the following structure:

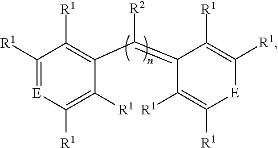

where each E is, at each occurrence in the compound, independently charged or neutral and is independently selected from S, Se, O, or Te, wherein at least one E is S or Se; each R¹ is, at each occurrence in the compound, independently selected from the group consisting of —H, C₁₋₈ alkyl group, halo group, —CN, aryl group, and heteroaryl group and adjacent R¹ groups can combine to form C₅₋₈ aryl groups, each R² is, at each occurrence in the compound, independently selected from the group consisting of —H, C₁₋₈ alkyl group, halo group, —CN, and aryl group, R² groups beta to each other can combine to form C₅₋₈ cycloalkyl groups, C₅₋₈ aryl groups or C₅₋₈ heteroaryl groups, and n is an odd number from 1 to 7; and Z is optionally present and is a counter ion.

In an embodiment, the compound does not have the following structures:

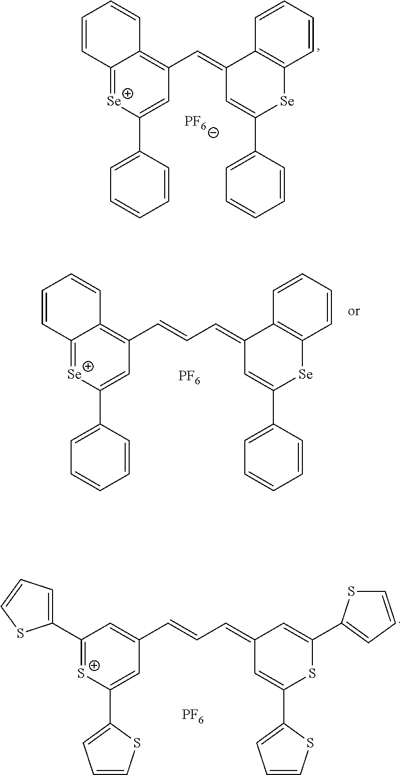

In an embodiment, the compound does not have the following structure:

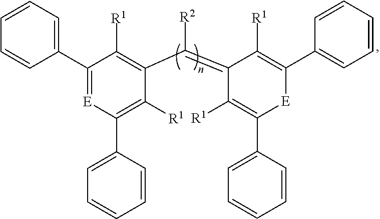

where E is S or Se. For example, the compound does not have the following structure:

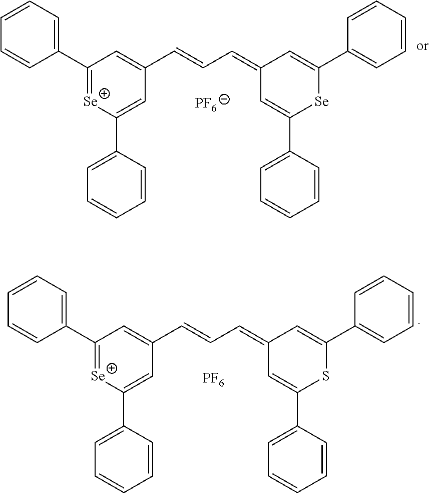

In various examples, the compounds have one of the following structures:

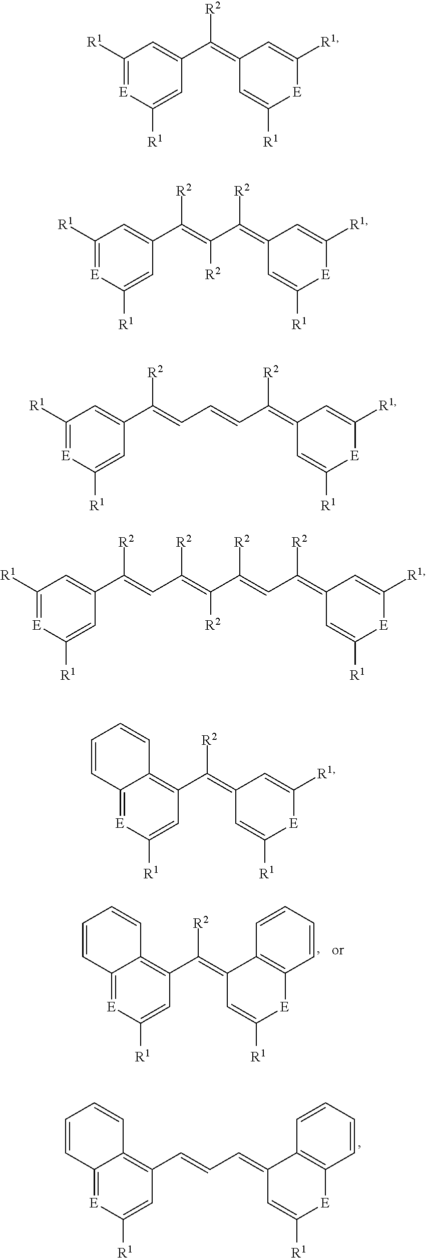

where R¹, R², and E are as defined herein.

In various examples, the compounds have one of the following structures:

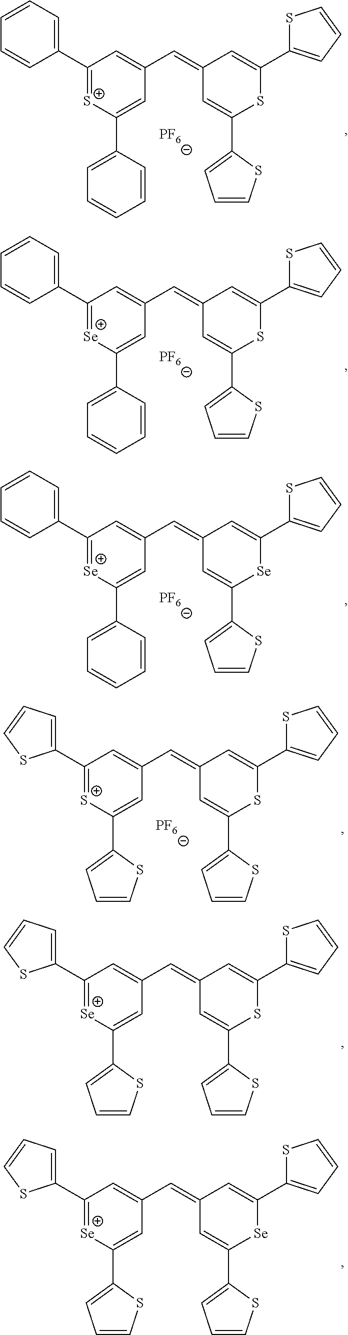

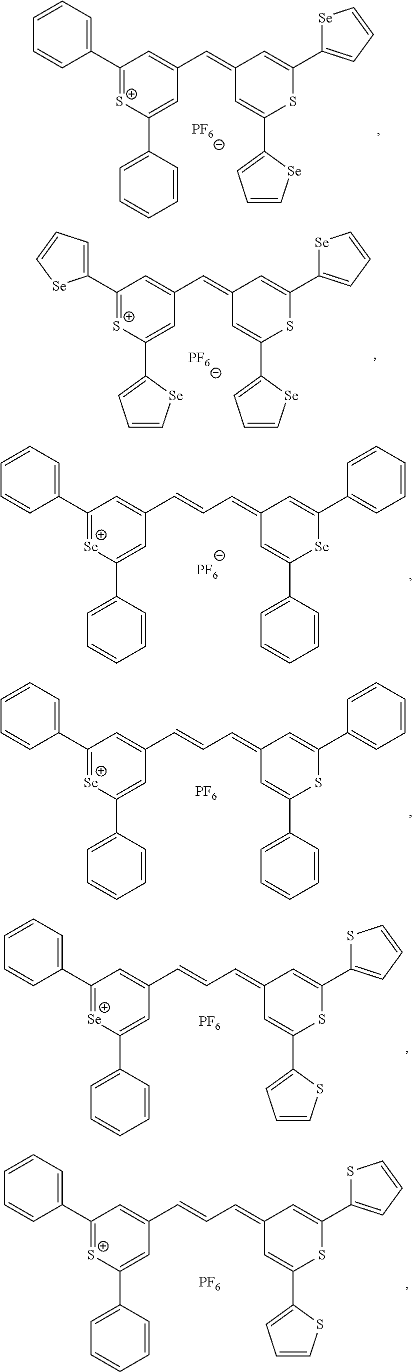

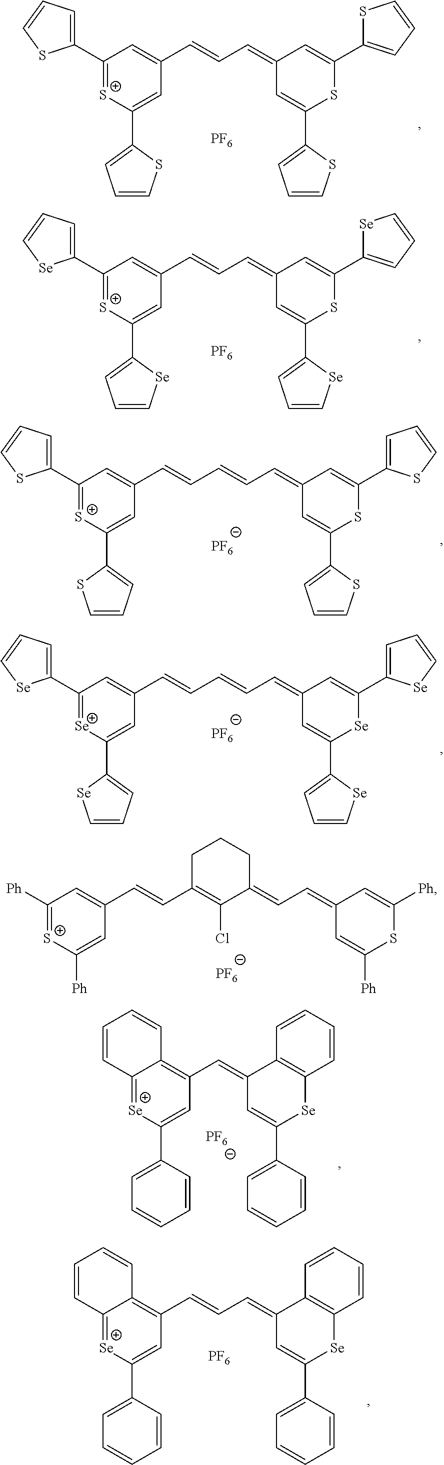

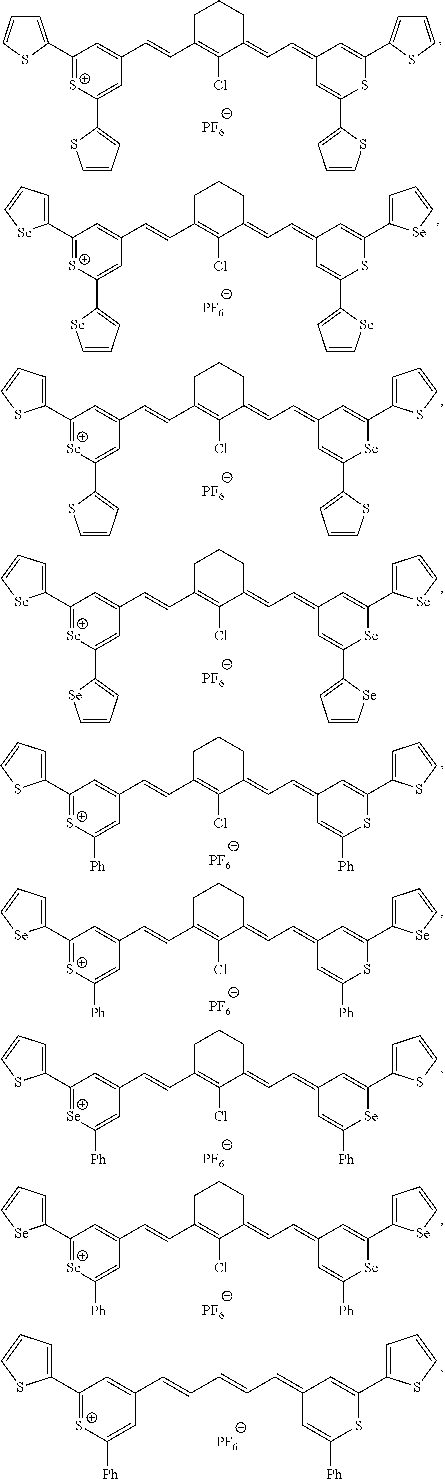

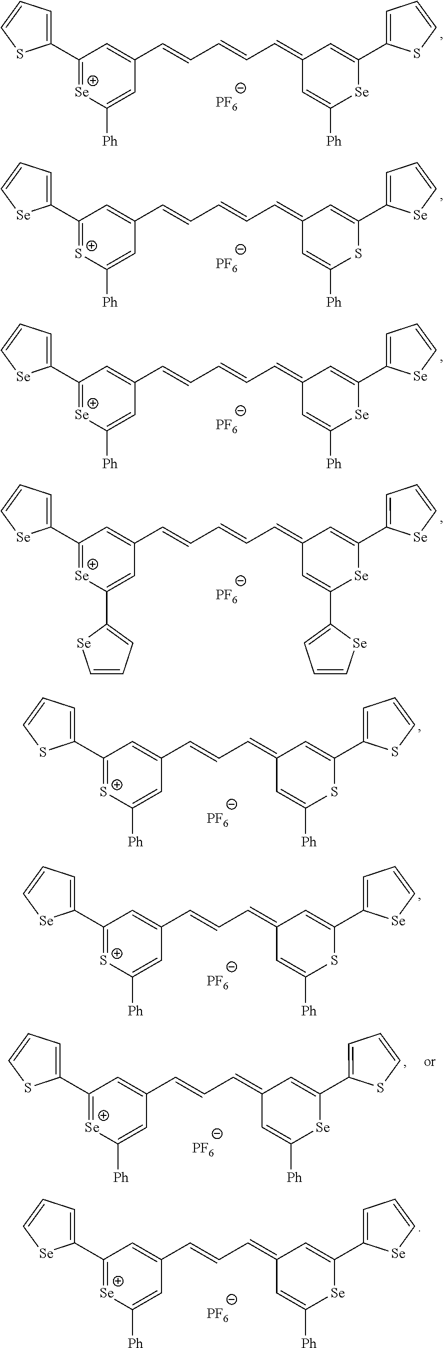

The present disclosure also provides composite nanostructures. The composite nanostructures can comprises: a core comprising a nanomaterial; one or more reporter molecules having the structure as described herein, wherein each of the reporter molecules is independently, at each occurrence in the composite nanostructure, directly covalently bound to the core or covalently bound to the core via a linking group to the core; and optionally, an encapsulating material that at least partially encapsulates the core and the one or more reporter molecules. For example, the core comprises a metal nanomaterial. For example, the core is a hollow gold nanoshell. The nanomaterial can be a nanoparticle and the nanoparticle size is 15 to 300 nm. The nanostructure morphology can be selected from the group consisting of sphere, rod, star, raspberry, and hollow shell. The encapsulating material can be an inorganic material, polyethylene glycol (PEG), or organic polymer.

The composite nanostructure can further comprise one or more targeting moieties bound (e.g., covalently or non-covalently bound) to the core or bound (e.g., covalently or non-covalently bound) to the core via a linking group. The encapsulating material, if present, at least partially encapsulates the core, the one or more reporter molecules. The one or more targeting moieties, if present, are directly bound (e.g., covalently or non-covalently bound) or bound (e.g., covalently or non-covalently bound) via a linking group to an outer surface of the encapsulating material. A targeting moiety is any moiety that specifically interacts with (e.g., binds) a target molecule. Examples of targeting moieties include, but are not limited to, antibodies, aptamers, synthetic receptors, DNA sequences, proteins, peptides, and the like. Examples of suitable conjugation methods and linkers are known in the art.

The present disclosure also provides methods of making composite nanostructures. For example, a method of making a composite nanostructure comprises binding one or more reporter molecules of the present invention to a core, and optionally, encapsulating the core and reporter molecule within an encapsulating material.

The present disclosure also provides methods of using the chalcogenopyrylium compounds or composite nanoparticles comprising the chalcogenopyrylium compounds. For example, a method for detecting one or more target molecules in a sample comprises: contacting an individual with one or more composite nanostructures; obtaining surface-enhanced Raman spectroscopy data (e.g., a surface-enhanced Raman spectrum) of a portion of the individual after contact of the portion of the individual with the one or more said composite nanostructures, wherein observation of surface-enhanced Raman spectroscopy data (e.g., a surface-enhanced Raman spectrum) attributable (e.g., specifically attributable) to a particular composite nanostructure of the one or more composite nanostructures indicates the presence of the target molecule in the portion of the individual corresponding to the targeting moiety of the particular nanostructure. The method may further comprise obtaining surface-enhanced Raman spectroscopy data (e.g., a surface-enhanced Raman spectrum) of one or more additional portions of the individual after contact of the one or more additional portions of the individual with the one or more of the composite nanostructures. The method may further comprise generating an image of at least a portion of the individual using the surface-enhanced Raman spectroscopy data (e.g., a surface-enhanced Raman spectrum) from the portion and, optionally, additional portions of the individual.

### BRIEF DESCRIPTION OF THE FIGURES

FIG. 1. X-ray crystal structure of dye 14 viewed from a) the top and b) from the side (rotated 90° from a)).

FIG. 2. The impact of 2-thienyl substituents on the intensity of SERS signals from dyes 9-13 on gold nanoparticles with 785-nm excitation. Gold nanoparticles were prepared via the addition of 7.5 ml 1% (w/v) sodium citrate to 1.0 L boiling 0.25 mM HAuCl₄.

FIG. 3. A comparison of the relative SERS intensity of aggregated and unaggregated dye 9-HGN assemblies with 1064-nm excitation.

FIGS. 4A through 4D. SERS spectra and structures of dyes 1-14 analyzed with HGNs (SPR recorded at 690 nm) and KCl. A laser excitation of 1064 nm and an exposure time of 5 seconds were employed in this analysis. All spectra have been background corrected.

FIG. 5. Structures of dyes 15-17. SERS spectra and structures of dye 15 and dye 16 analyzed with HGNs (SPR recorded at 690 nm) and KCl. A laser excitation of 1064 nm and an exposure time of 5 seconds were employed in this analysis. All spectra have been background corrected.

FIGS. 6A through 6C. SERS particle dilution study for dyes 9, 11-13 and the commercial dyes BPE and AZPY with HGNs and KCl over the concentration range 1.3 nM to 1 pM. The peak height at 1590 cm⁻¹ was analyzed by subtracting the background ‘HGN only’ signal from each data point. Error bars represent one standard deviation resulting from 3 replicate samples and 5 scans of each using an excitation wavelength of 1064 nm and an exposure time of 5 seconds.

FIG. 7. SERS spectra and structures of dyes 1-14 analyzed with HGNs (SPR recorded at 720 nm) and KCl. A laser excitation of 1280 nm and an exposure time of 7 seconds were employed in this analysis, with the exception of dye 14 which had an acquisition time of 3 seconds. All spectra have been background corrected.

FIG. 8. SERS particle dilution study for dye 13 with HGNs and KCl over the concentration range 1.3 nM to 80 pM. The peak height at 1590 cm⁻¹ was analyzed by subtracting the background ‘HGN only’ signal from each data point. Error bars represent one standard deviation resulting from 3 replicate samples and 5 scans of each using an excitation wavelength of 1280 nm and an exposure time of 7 seconds. The LOD was calculated to be 11.5 pM.

FIG. 9. Unaggregated SERS spectra of dyes 13 and 14 analysed with HGNs (SPR recorded at 720 nm) and deionised water. A laser excitation of 1280 nm and an exposure time of 7 seconds were employed in this analysis. All spectra have been background corrected.

FIGS. 10A through 10C. Comparison of the SERS response with 1280-nm excitation for: (A) dye 14 on hollow gold nanoshells (HGN), solid gold (AuNP) and solid silver (AgNP) nanoparticles. An exposure time of 1 second (for dye 14 on HGN) and 7 seconds (for dye 14 on AuNP and AgNP) were employed in this analysis; (B) dye 13 on HGN, AuNP and AgNP. An exposure time of 3 seconds (for dye 13 on HGN) and 7 seconds (for dye 13 on AuNP and AgNP) were employed in this analysis; and (C) dye 8 on HGN, AuNP and AgNP. An exposure time of 3 seconds (for dye 8 on HGN) and 7 seconds (for dye 8 on AuNP and AgNP) were employed in this analysis.

FIG. 11. A comparison of the SERS response with 1280-nm excitation for dye 12 on solid gold (AuNP) and silver nanoparticles (AgNP), not HGNs, and the SERS response of the commercially available dyes BPE (bis(4-pyridyl)ethylene) and AZPY (4,4′-azopyridine) on hollow gold nanoshells (HGN).

FIG. 12. Construction of nanoparticle assembly for use in biological imaging applications.

FIG. 13. A comparison of the relative SERS intensity of aggregated and unaggregated dye 20-HGN assemblies with 1064-nm excitation.

FIG. 14. Reagents and conditions: (a) (PhSe)₂ (0.5 equiv), NaBH₄ (2.0 equiv), THF, EtOH, 10 min; (b) 3 M KOH (aq), reflux, 15 h (h=hour(s)); (c) P₂O₅, CH₃SO₃H, 65° C., 5 min; (d) MeMgBr (3.0 equiv), THF, rt, 30 min; (e) 10% HPF₆ (aq), rt, 30 min; (f) Ac₂O, 105° C., 5 min (min=minute(s)).

FIG. 15. Reagents Conditions (a) N,N-dimethylthioformamide (3.0 equiv), Ac₂O, 1 h, 95° C.; (d) satd. NaHCO₃, CH₃CN, 45 min, 40-80° C.; (e) Ac₂O, 5-10 min, 105° C.

FIG. 16. Synthesis and structure of the SERRS-reporters and SERRS-nanoprobe. (A) Reaction scheme for the synthesis of pyrylium-based SERRS-reporters (1a-3). (B) A 60-nm gold core encapsulated in a 15 nm thick chalcogenopyrylium dye containing silica shell. The structure, yields, and optical properties of the different chalcogenopyrylium-based Raman reporters are shown in the table.

FIG. 17. The effect of the counterion on colloidal stability. (A) The effect of the counterion (Z) on SERRS intensity (785-nm, 50 μW/cm², 1.0-s acquisition time, 5× objective). (B) Effect of counterion on the colloidal stability of CP-dye 1a-based SERRS-nanoprobes (n=3, error bars represent standard deviations).

FIG. 18. The SERRS-intensity as a function of dye affinity for the gold surface. (A) Molecular structures of the adsorbed CP-dyes (1a-3) arranged by increased number of 2-thienyl substituents. (B) SERRS spectra of the CP-based SERRS-nanoprobes. The SERRS spectra were baseline corrected to allow proper comparison. Insert: intensity of the 1600 cm⁻¹ peak (n=3; error bars represent standard deviations, *P<0.05; an unpaired Student's t-test was performed). C) Colloidal stability of the CP-based SERRS-nanoprobes as determined by LSPR measurements (n=3; error bars represent standard deviations).

FIG. 19. Comparison of the SERRS-signal intensity of the optimized CP-dye 3 versus a widely used resonant dye IR792. (A) Structure of the resonant dye IR792 and chalcogenopyrylium dye 3. (B) SERRS intensity of an equimolar amount of an IR792-based SERRS-nanoprobe and a 3-based SERRS-nanoprobe that were synthesized of an equimolar amount of the dyes. (C) Limits of detection of the IR792-(cyan) and 3 [symbol] (red) based SERRS-nanoprobes were performed in triplicate and determined to be 1.0 fM and 100 attomolar, respectively.

FIG. 20. Comparison between EGFR-targeted IR792- or 3-based SERRS-nanoprobes in an A431 tumor xenograft. Female nude mice (n=5) bearing A431 xenograft tumors were injected intravenously via tail vein with an equimolar amount of EGFR-antibody (cetuximab)-conjugated IR792- and CP 3-based SERRS-nanoprobes (15 fmol/g per probe; total injected dose: 30 fmol/g). After 18 hours, the tumors were imaged in situ by Raman (10 mW/cm², 1.5 s acquisition time, 5× objective). The chalcogenopyrylium dye 3-based SERRS-nanoprobe (red) provided ˜3× more contrast than the IR792-based SERRS-nanoprobe (cyan) (22.442 cps/cm² versus 7.313 cps/cm², respectively). All scale bars represent 2.0 mm.

FIG. 21. Immunohistochemistry and ex-vivo Raman imaging of the A431 tumor. The excised tumor was scanned by Raman imaging (10 mW/cm², 1.5 s acquisition time, 5× objective) and subsequently fixed in 4% paraformaldehyde and processed for H&E staining and anti-EGFR immunohistochemistry. With the exception of a hypointense Raman region in the center of the tumor, the tumor homogenously expressed EGFR and the EGFR-targeted SERRS-nanoprobes had accumulated throughout the tumor. The hypointense Raman area corresponds to a highly necrotic region within the tumor, which explains the lack of SERRS-nanoprobe accumulation and decreased Raman signal. All scale bars represent 1.0 mm.

FIG. 22. SERS spectrum and structure of dye 14 analyzed with HGNs (SPR recorded at 720 nm) and KCl. A laser excitation of 1280 nm and an exposure time of 3 seconds were employed in this analysis. The spectrum has been background corrected.

FIG. 23. SERS particle dilution study for dye 14 with HGNs and KCl over the concentration range 1.93 nM to 6 pM. The limit of detection was calculated to be 1.47 pM. The peak height at 1590 cm⁻¹ was analysed by subtracting the background ‘HGN only’ signal from each data point. Error bars represent one standard deviation resulting from 3 replicate samples and 5 scans of each using an excitation wavelength of 1280 nm and an exposure time of 7 seconds.

FIG. 24. PCA scores plot discriminating between each of the 14 chalcogenopyrylium dyes and grouping them according to their structures and SERS spectra. The red cluster contains the trimethine dyes 9-14 which produce the best SERS signals, blue cluster highlights the monomethine dyes (1-3,5,7-8) which work well as reporters for SERS and the green clustering contains the two dyes which didn't produce any signal with HGNs (dyes 4 and 6).

### DETAILED DESCRIPTION OF THE DISCLOSURE

As used herein, the term “alkyl group”, unless otherwise stated, refers to branched or unbranched hydrocarbons. Examples of such alkyl groups include methyl groups, ethyl groups, propyl groups, butyl groups, isopropyl groups, tert-butyl groups, and the like. For example, the alkyl group can be a C₁-C₈ alkyl group including all integer numbers of carbons and ranges of numbers of carbons there between. The alkyl group can be unsubstituted or substituted with various substituents.

As used herein, the term “aryl group”, unless otherwise stated, refers to a C₅-C₈ aromatic carbocyclic group including all integer numbers of carbons and ranges of numbers of carbons there between. The aryl group can be unsubstituted or substituted with various substituents (e.g., as described herein) which may be the same or different. A non-limiting example of a suitable aryl group include phenyl.

As used herein, the term “halo group”, unless otherwise state, means fluoro, chloro, bromo, or iodo group. As used herein, the term “halide”, unless otherwise state, means fluoride, chloride, bromide, or iodide.

As used herein, the term “heteroaryl group”, unless otherwise stated, refers to a C₅-C₈ monocyclic or fused bicyclic ring system, including all integer numbers of carbons and ranges of numbers of carbons there between, wherein 1-8 of the ring atoms are selected from the group consisting of S, Se, O, P, B, and N. The heteroaryl group can be unsubstituted or substituted with various substituents (e.g., as described herein) which may be the same or different. Examples of heteroaryl groups include, benzofuranyl, thienyl, furyl, pyridyl, oxazolyl, quinolyl, thiophenyl, selenophenyl, isoquinolyl, indolyl, triazinyl, triazolyl, isothiazolyl, isoxazolyl, imidazolyl, benzothiazolyl, pyrazinyl, pyrimidinyl, thiazolyl, and thiadiazolyl groups.

As used herein, the term “cycloalkyl group”, unless otherwise stated, refers to a C₅-C₈ cyclic aliphatic group, including all integer numbers of carbons and ranges of numbers of carbons there between. Examples of cycloalkyl groups include cyclohexyl, cyclohexenyl, and cyclopentyl groups. The cycloalkyl group can be unsubstituted or substituted with various substituents.

It is an object of the present disclosure to provide surface-enhanced Raman spectroscopic (SERS) or surface-enhanced resonance Raman scattering (SERRS) active composite nanostructures, methods of fabricating these nanostructures, and methods of using these nanostructures. It is to be understood that references to SERS in this application include SERRS.

The surface-enhanced Raman spectroscopic (SERS) active composite nanostructures are comprised of a core attached (e.g., covalently or non-covalently) to at least one reporter molecule, and, optionally, an encapsulating material (i.e. a shell). The reporter molecule(s) is (are) bonded to the core directly or via a coupling agent. The reporter molecule(s) is (are) selected from the chalcogenopyrylium dyes described herein. In some embodiments, at least two distinct reporter molecules may be bonded to the core, thus allowing for detection of more than one SERS signal. The encapsulating material is disposed over the core and the reporter molecule. The reporter molecule, whether or not encapsulated, has a measurable surface-enhanced Raman spectroscopic signature. Although not intending to be bound by theory, the core optically enhances the SERS spectrum, while the reporter molecule provides a distinct spectroscopic SERS signature. Although optional, disposing the encapsulant material over the core and reporter molecule does not substantially impact the spectroscopic SERS signature of the reporter molecule, while protecting the core and reporter molecules. A preferred size range for nanoparticles is 50-100 nm, but particles in the range of 40-300 nm are also useful.

The core can be made of plasmonic materials that have a resonance in the range of 400 nm to 2000 nm, including all nm values and ranges therebetween. In an example, the plasmonic materials have a resonance in the range of 780 nm to 1600 nm. In an example, the plasmonic materials have a resonance in the range of 1000 nm to 1600 nm (e.g., 1064 nm or 1280 nm). The core can be made of nanomaterials such as, but not limited to, metals. In some embodiments, the core can be a metallic core. In particular, the core can be made of noble metals such as, but not limited to, gold, silver, copper, and combinations thereof. In other embodiments, the core can be metal-coated silica particles such as gold-coated silica particles. Suitable morphologies for such materials include, but are not limited to, spheres, rods, stars, raspberries, and hollow shells. In an embodiment, the core can be a gold core. In some examples, the core is a hollow gold nanoshell. The core can be a nanomaterial, such as, for example, a nanoparticle, and the core can have a size (e.g., longest dimension), which can be measured by electron microscopy, of 15 nm to 300 nm, including all nm values and ranges therebetween. For example, the core has a size of 40 nm to 100 nm.

Suitable encapsulating materials, if used, include but are not limited to, silica-based materials such as xerogels from tetraalkoxy silanes or organically modified xerogels from organotrialkoxy silanes and tetraalkoxy silanes; polyethylene glycol (PEG) such as PEG 500; and organic polymers such as, but not limited to, polyvinylethylene (PVE) and polyvinylpropylene (PVP). The encapsulating materials can be inorganic materials including, but not limited to, SiO₂ or MnO₂.

The surface-enhanced Raman spectroscopic (SERS) active composite nanostructures of the present disclosure may further comprise a coupling agent, wherein the coupling agent is bonded to the core and reporter molecule. An example of a suitable coupling agent is thiol PEG with carboxylate terminal groups.

The surface-enhanced Raman spectroscopic (SERS) active composite nanostructures of the present disclosure can be incorporated into (e.g., used in) systems such as, for example, anti-counterfeit systems, covert tagging systems, cytometry systems (e.g., a flow cytometry system), chemical array systems, biomolecule array systems, biosensing systems, bioimaging systems, biolabeling systems, high-speed screening systems, gene expression systems, protein expression systems, medical diagnostic systems, diagnostic libraries, and microfluidic systems.

It is also an object of the present disclosure to provide chalcogenopyrylium compounds. The chalcogenopyrylium compounds can be dyes that can be used as reporter molecules for surface-enhanced Raman scattering (SERS) attached to nanoparticles such as noble metal nanoparticles, for example, those comprised of gold, silver, copper or combinations thereof. It is an advantage that SERS active composite nanostructures comprising the SERS reporters of this disclosure work with excitation from light sources emitting in the near infrared region of 1000 to 1600 nm. For example, SERS reporters of the present disclosure bound to noble metal nanoparticles such as hollow gold nanoparticles work with excitation from light sources emitting in the near infrared region of 1000 to 1600 nm, for example, both/either 1064-nm and/or 1280-nm lasers.

The present disclosure also provides novel chalcogenopyrylium compositions of matter as SERS reporters attached to nanoparticles such as noble metal nanoparticles (e.g., those comprised of gold, silver, copper or combinations thereof). It is an advantage that SERS active composite nanostructures comprising novel chalcogenopyrylium compositions of matter of this disclosure work with excitation from light sources emitting in the near infrared region of 1000 to 1600 nm. For example, novel chalcogenopyrylium compositions of matter of this disclosure bound to noble metal nanoparticles such as hollow gold nanoparticles work with excitation from light sources emitting in the near infrared region of 1000 to 1600 nm, for example, both/either 1064-nm and/or 1280-nm lasers.

The chalcogenopyryliums of the present disclosure can be defined by the following generic structures:

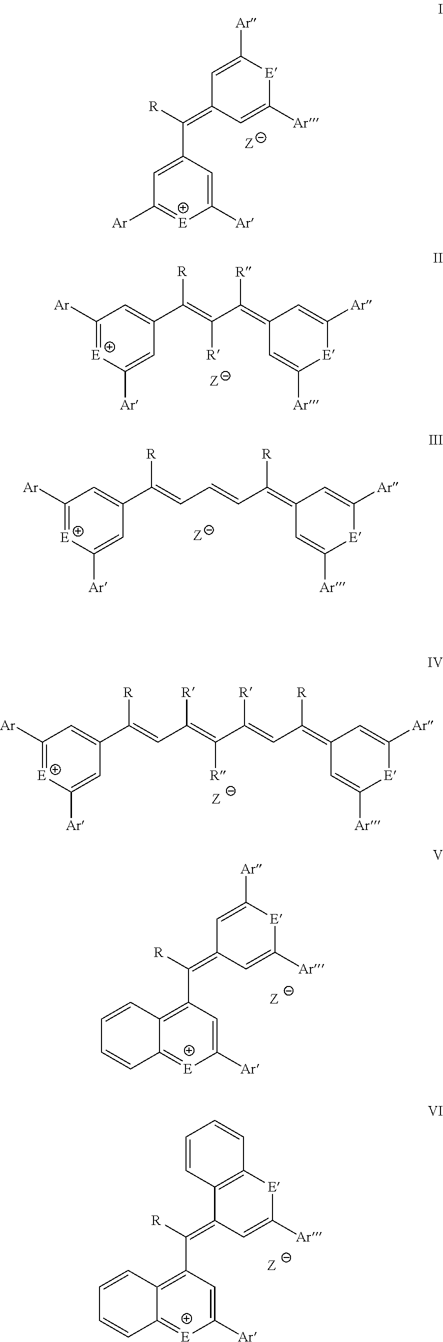

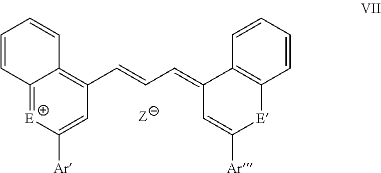

In one embodiment, the present disclosure provides thiopyrylium dyes and selenopyrylium dyes of general structures I-VII (shown above) as SERS reporters attached to nanoparticles, for example, those comprised of gold (e.g. hollow gold nanoshells), silver, copper or combinations thereof, and methods of using these compositions for SERS and/or SERSS spectroscopy with incident light from 780 nm to 1300 nm wherein E and E′ are independently selected from the chalcogen atoms 0, S, Se, and Te wherein at least one of E or E′ is S or Se; Ar, Ar′, Ar″, and Ar′″ are independently selected from the group consisting of phenyl (substituted or unsubstituted), 2-thienyl (substituted or unsubstituted), 3-thienyl (substituted or unsubstituted), 2-selenophenyl (substituted or unsubstituted), and 3-selenophenyl (substituted or unsubstituted); and the counter ion Z is an anion. In other embodiments of the composition and method, the counter ion Z is selected from the group consisting of PF₆, BF₄, Cl, Br, CF₃CO₂, and CF₃SO₃.

In another embodiment, the present disclosure provides thiopyrylium dyes and selenopyrylium dyes of general structures I-VII as SERS reporters attached to noble metal nanoparticles such as those comprised of gold (e.g. hollow gold nanoparticles), silver, copper, or combinations thereof and methods of using these compositions for SERS and/or SERSS spectroscopy with incident light from a 1280-nm laser wherein E and E′ are independently selected from the chalcogen atoms 0, S, Se, and Te wherein at least one of E or E′ is S or Se; Ar, Ar′, Ar″, and Ar′″ are independently selected the group consisting of phenyl (substituted or unsubstituted), 2-thienyl (substituted or unsubstituted), 3-thienyl (substituted or unsubstituted), 2-selenophenyl (substituted or unsubstituted), and 3-selenophenyl (substituted or unsubstituted); and the counter ion Z is an anion selected from the group consisting of PF₆, BF₄, Cl, Br, CF₃CO₂, and CF₃SO₃.

In another embodiment, the present disclosure provides thiopyrylium dyes and selenopyrylium dyes of general structures I-III and V-VII as SERS reporters attached to nanoparticles, for example, those comprised of gold (e.g. hollow gold nanoshells), silver, copper or combinations thereof, and methods of using these compositions for SERS and/or SERSS spectroscopy with incident light from 780 nm to 1600 nm wherein E and E′ are independently selected from the chalcogen atoms 0, S, Se, and Te wherein at least one of E or E′ is S or Se; Ar, Ar′, Ar″, and Ar′″ are independently selected from the group consisting of phenyl (substituted or unsubstituted), 2-thienyl (substituted or unsubstituted), 3-thienyl (substituted or unsubstituted), 2-selenophenyl (substituted or unsubstituted), 3-selenophenyl (substituted or unsubstituted); R, R′, and R″ are independently selected from the group consisting of H, C₁₋₈ alkyl (straight chain or branched), halides and pseudohalides; and the counter ion Z is an anion selected from Z═PF₆, BF₄, Cl, Br, CF₃CO₂, and CF₃SO₃. In one embodiment, R, R′, and R″ are independently selected from the group consisting of H, C₁₋₈ alkyl (straight chain or branched), Cl, Br and CN.

In another embodiment, the present disclosure provides thiopyrylium dyes and selenopyrylium dyes of general structures I-III and V-VII as SERS reporters attached to noble metal nanoparticles such as those comprised of gold (e.g. hollow gold nanoparticles), silver, copper, or combinations thereof and methods of using these compositions for SERS and/or SERSS spectroscopy with incident light from a 1280-nm laser wherein E and E′ are independently selected from the chalcogen atoms 0, S, Se, and Te wherein at least one of E or E′ is S or Se; Ar, Ar′, Ar″, and Ar′″ are independently selected from the group consisting of phenyl (substituted or unsubstituted), 2-thienyl (substituted or unsubstituted), 3-thienyl (substituted or unsubstituted), 2-selenophenyl (substituted or unsubstituted), and 3-selenophenyl (substituted or unsubstituted); R, R′, and R″ are independently selected from the group consisting of H, C₁₋₈ alkyl (straight chain or branched), halides and pseudohalides; and the counter ion Z is an anion selected from the group consisting of PF₆, BF₄, Cl, Br, CF₃CO₂, and CF₃SO₃. In one embodiment, R, R′, and R″ are independently selected from the group consisting of H, C₁₋₈ alkyl (straight chain or branched), Cl, Br and CN.

In another embodiment, the present disclosure provides thiopyrylium dyes and selenopyrylium dyes of general structures I-III and V-VII as SERS reporters attached to nanoparticles, for example, those comprised of gold (e.g. hollow gold nanoshells), silver, copper or combinations thereof, and methods of using these compositions for SERS and/or SERSS spectroscopy with incident light from 780 nm to 1600 nm wherein E and E′ are independently selected from the chalcogen atoms S and Se; Ar, Ar′, Ar″, and Ar′″ are independently selected from the group consisting of phenyl (substituted or unsubstituted), 2-thienyl (substituted or unsubstituted), 3-thienyl (substituted or unsubstituted), 2-selenophenyl (substituted or unsubstituted), and 3-selenophenyl (substituted or unsubstituted); R, R′, and R″ are independently selected from the group consisting of H, C₁₋₈ alkyl (straight chain or branched), halides or pseudohalides; and the counter ion Z is an anion selected from the group consisting of PF₆, BF₄, Cl, Br, CF₃CO₂, and CF₃SO₃.

In another embodiment, the present disclosure provides thiopyrylium dyes and selenopyrylium dyes of general structures I-III and V-VII as SERS reporters attached to noble metal nanoparticles such as those comprised of gold (e.g. hollow gold nanoparticles), silver, copper, or combinations thereof and methods of using these compositions for SERS and/or SERSS spectroscopy with incident light from a 1280-nm laser wherein E and E′ are independently selected from the chalcogen atoms S and Se; Ar, Ar′, Ar″, and Ar′″ are independently selected from the group consisting of phenyl (substituted or unsubstituted), 2-thienyl (substituted or unsubstituted), 3-thienyl (substituted or unsubstituted), 2-selenophenyl (substituted or unsubstituted), 3-selenophenyl (substituted or unsubstituted); R, R′, and R″ are independently selected from the group consisting of H, C₁₋₈ alkyl (straight chain or branched), halides and pseudohalides; and the counter ion Z is an anion selected from the group consisting of PF₆, BF₄, Cl, Br, CF₃CO₂, and CF₃SO₃. In one embodiment, R, R′, and R″ are independently selected from the group consisting of H, C₁₋₈ alkyl (straight chain or branched), Cl, Br and CN.

In another embodiment, the present disclosure provides thiopyrylium dyes and selenopyrylium dyes of general structures I-III and V-VII as SERS reporters attached to nanoparticles, for example, those comprised of gold (e.g. hollow gold nanoshells), silver, copper or combinations thereof, and methods of using these compositions for SERS and/or SERSS spectroscopy with incident light from 780 nm to 1600 nm wherein E and E′ are independently selected from the chalcogen atoms S and Se; Ar, Ar′, Ar″, and Ar′″ are independently selected from the group consisting of phenyl (substituted or unsubstituted), 2-thienyl (substituted or unsubstituted), and 2-selenophenyl (substituted or unsubstituted); R, R′, and R″ are independently selected from the group consisting of H, C₁₋₈ alkyl (straight chain or branched), halides and pseudohalides; and the counter ion Z is an anion selected from the group consisting of PF₆, BF₄, Cl, Br, CF₃CO₂, and CF₃SO₃. In one embodiment, R, R′, and R″ are independently selected from the group consisting of H, C₁₋₈ alkyl (straight chain or branched), Cl, Br and CN.

In another embodiment, the present disclosure provides thiopyrylium dyes and selenopyrylium dyes of general structures I-III and V-VII as SERS reporters attached to noble metal nanoparticles such as those comprised of gold (e.g. hollow gold nanoparticles), silver, copper, or combinations thereof and methods of using these compositions for SERS and/or SERSS spectroscopy with incident light from a 1280-nm laser wherein E and E′ are independently selected from the chalcogen atoms S and Se; Ar, Ar′, Ar″, and Ar′″ are independently selected from the group consisting of phenyl (substituted or unsubstituted), 2-thienyl (substituted or unsubstituted), and 2-selenophenyl (substituted or unsubstituted); R, R′, and R″ are independently selected from the group consisting of H, C₁₋₈ alkyl (straight chain or branched), halides and pseudohalides; and the counter ion Z is an anion selected from the group consisting of PF₆, BF₄, Cl, Br, CF₃CO₂, and CF₃SO₃. In one embodiment, R, R′, and R″ are independently selected from the group consisting of H, C₁₋₈ alkyl (straight chain or branched), Cl, Br and CN.

In another embodiment, the present disclosure provides thiopyrylium dyes and selenopyrylium dyes of general structures I-III and V-VII as SERS reporters attached to nanoparticles, for example, those comprised of gold (e.g. hollow gold nanoshells), silver, copper or combinations thereof, and methods of using these compositions for SERS and/or SERSS spectroscopy with incident light from 780 nm to 1600 nm wherein E and E′ are independently selected from the chalcogen atoms S and Se; Ar, Ar′, Ar″, and Ar′″ are independently selected from the group consisting of phenyl, 2-thienyl, and 2-selenophenyl; R R′, and R″ are independently selected from the group consisting of H, C₁₋₈ alkyl (straight chain or branched), halides or pseudohalides; and the counter ion Z is an anion selected from the group consisting of PF₆, BF₄, Cl, Br, CF₃CO₂, and CF₃SO₃. In one embodiment, R, R′, and R″ are independently selected from the group consisting of H, C₁₋₈ alkyl (straight chain or branched), Cl, Br and CN.

In another embodiment, the present disclosure provides thiopyrylium dyes and selenopyrylium dyes of general structures I-III and V-VII as SERS reporters attached to noble metal nanoparticles such as those comprised of gold (e.g. hollow gold nanoparticles), silver, copper, or combinations thereof and methods of using these compositions for SERS and/or SERSS spectroscopy with incident light from a 1280-nm laser wherein E and E′ are independently selected from the chalcogen atoms S and Se; Ar, Ar′, Ar″, and Ar′″ are independently selected from the group consisting of phenyl, 2-thienyl, and 2-selenophenyl; R, R′, and R″ are independently selected from the group consisting of H, C₁₋₈ alkyl (straight chain or branched), halides or pseudohalides; and the counter ion Z is an anion selected from the group consisting of PF₆, BF₄, Cl, Br, CF₃CO₂, and CF₃SO₃. In one embodiment, R, R′, and R″ are independently selected from the group consisting of H, C₁₋₈ alkyl (straight chain or branched), Cl, Br and CN.

In another embodiment, the present disclosure provides thiopyrylium dyes and selenopyrylium dyes of general structures I-III and V-VII as SERS reporters attached to nanoparticles, for example, those comprised of gold (e.g. hollow gold nanoshells), silver, copper or combinations thereof, and methods of using these compositions for SERS and/or SERSS spectroscopy with incident light from 780 nm to 1600 nm wherein E and E′ are independently selected from the chalcogen atoms S and Se; Ar, Ar′, Ar″, and Ar′″ are independently selected from the group consisting of phenyl, 2-thienyl, and 2-selenophenyl; R, R′ and R″ are H; and the counter ion Z is an anion selected from the group consisting of PF₆, BF₄, Cl, Br, CF₃CO₂, and CF₃SO₃.

In another embodiment, the present disclosure provides thiopyrylium dyes and selenopyrylium dyes of general structures I-III and V-VII as SERS reporters attached to noble metal nanoparticles such as those comprised of gold (e.g. hollow gold nanoparticles), silver, copper, or combinations thereof and methods of using these compositions for SERS and/or SERSS spectroscopy with incident light from a 1280-nm laser where E and E′ are independently selected from the chalcogen atoms S and Se; Ar, Ar′, Ar″, and Ar′″ are independently selected from the group consisting of phenyl, 2-thienyl, and 2-selenophenyl; R, R′ and R″ are H; and the counter ion Z is an anion selected from the group consisting of PF₆, BF₄, Cl, Br, CF₃CO₂, and CF₃SO₃.

In another embodiment, the present disclosure provides thiopyrylium dyes and selenopyrylium dyes of general structures I-III and V-VII as SERS reporters attached to nanoparticles, for example, those comprised of gold (e.g. hollow gold nanoshells), silver, copper or combinations thereof and methods of using these compositions for SERS and/or SERSS spectroscopy with incident light from 780 nm to 1600 nm wherein E and E′ are independently selected from the chalcogen atoms S and Se; Ar, Ar′, Ar″, and Ar′″ are independently selected from the group consisting of phenyl, 2-thienyl, and 2-selenophenyl; R, R′ and R″ are H; and Z is PF₆.

In another embodiment, the present disclosure provides thiopyrylium dyes and selenopyrylium dyes of general structures I-III and V-VII as SERS reporters attached to noble metal nanoparticles such as those comprised of gold (e.g. hollow gold nanoparticles), silver, copper, or combinations thereof and methods of using these compositions for SERS and/or SERSS spectroscopy with incident light from, a 1280-nm laser wherein E and E′ are independently selected from the chalcogen atoms S and Se; Ar, Ar′, Ar″, and Ar′″ are independently selected from the group consisting of phenyl, 2-thienyl, and 2-selenophenyl; R, R′ and R″ are H; and Z is PF₆.

In another embodiment, the present disclosure provides thiopyrylium dyes and selenopyrylium dyes of general structures I-III wherein E and E′ are independently selected from the chalcogen atoms S and Se; Ar, Ar′, Ar″, and Ar′″ are independently selected from the group consisting of phenyl, 2-thienyl, and 2-selenophenyl wherein at least two of the groups Ar, Ar′, Ar″, or Ar′″ are 2-thienyl or 2-selenophenyl; R, R′ and R″ are H; and Z is selected from the group consisting of PF₆, BF₄, Cl, Br, CF₃CO₂, and CF₃SO₃, which are novel compositions of matter. The subject disclosure also provides thiopyrylium dyes and selenopyrylium dyes of general structures I-III as SERS reporters attached to, for example, hollow gold, silver or copper nanoparticles and methods of using these compositions for SERS and/or SERSS spectroscopy with incident light from 780 nm to 1600 nm, wherein E and E′ are independently selected from the chalcogen atoms S and Se; Ar, Ar′, Ar″, and Ar′″ are independently selected from the group consisting of phenyl, 2-thienyl, and 2-selenophenyl wherein at least two of the groups Ar, Ar′, Ar″, or Ar′″ are 2-thienyl or 2-selenophenyl; R, R′ and R″ are H; and Z is selected from the group consisting of PF₆, BF₄, Cl, Br, CF₃CO₂, and CF₃SO₃. In other embodiments of the composition and the method, the incident light is from a 1280-nm laser.

In another embodiment, the present disclosure provides thiopyrylium dyes and selenopyrylium dyes of general structures I-III wherein E and E′ are independently selected from the chalcogen atoms S and Se; Ar, Ar′, Ar″, and Ar′″ are independently selected from the group consisting of phenyl, 2-thienyl, and 2-selenophenyl wherein at least two of the groups Ar, Ar′, Ar″, or Ar′″ are 2-thienyl or 2-selenophenyl; R, R′ and R″ are H; and Z is PF₆⁻, which are novel compositions of matter. The subject disclosure also provides thiopyrylium dyes and selenopyrylium dyes of general structures I-III as SERS reporters attached to, for example, hollow gold, silver or copper nanoparticles and methods of using these compositions for SERS and/or SERSS spectroscopy with incident light from 780 nm to 1600 nm, wherein E and E′ are independently selected from the chalcogen atoms S and Se; Ar, Ar′, Ar″, and Ar′″ are independently selected from the group consisting of phenyl, 2-thienyl, and 2-selenophenyl wherein at least two of the groups Ar, Ar′, Ar″, or Ar′″ are 2-thienyl or 2-selenophenyl; R, R′ and R″ are H; and Z is PF₆. In other embodiments of the composition and the method, the incident light is from a 1280-nm laser.

In another embodiment, the present disclosure provides thiopyrylium dyes and selenopyrylium dyes of general structure IV as SERS reporters attached to nanoparticles, for example, those comprised of gold (e.g. hollow gold nanoshells), silver, copper or combinations thereof, and methods of using these compositions for SERS and/or SERSS spectroscopy with incident light from 1000 nm to 1300 nm wherein E and E′ are independently selected from the chalcogen atoms 0, S, Se, and Te wherein at least one of E or E′ is S or Se; Ar, Ar′, Ar″, and Ar′″ are independently selected from the group consisting of phenyl (substituted or unsubstituted), 2-thienyl (substituted or unsubstituted), 3-thienyl (substituted or unsubstituted), 2-selenophenyl (substituted or unsubstituted), and 3-selenophenyl (substituted or unsubstituted); R is H; all R′s are H or together can form a five- or six-membered ring; R″ is selected from the group consisting of H, halides, pseudohalides alkylthio and arylthio groups; and the counter ion Z is an anion selected from the group consisting of PF₆, BF₄, Cl, Br, CF₃CO₂, and CF₃SO₃. In one embodiment, R″ is selected from the group consisting of H, Cl, Br, CN, alkylthio and arylthio groups.

In another embodiment, the present disclosure provides thiopyrylium dyes and selenopyrylium dyes of general structure IV as SERS reporters attached to nanoparticles, for example, those comprised of gold (e.g. hollow gold nanoshells), silver, copper or combinations thereof, and methods of using these compositions for SERS and/or SERSS spectroscopy with incident light from a 1280-nm laser wherein E and E′ are independently selected from the chalcogen atoms 0, S, Se, and Te wherein at least one of E or E′ is S or Se; Ar, Ar′, Ar″, and Ar′″ are independently selected from the group consisting of phenyl (substituted or unsubstituted), 2-thienyl (substituted or unsubstituted), 3-thienyl (substituted or unsubstituted), 2-selenophenyl (substituted or unsubstituted), and 3-selenophenyl (substituted or unsubstituted); R is H; all R′s are H or together can form a five- or six-membered ring; R″ is selected from the group consisting of H, halides, pseudohalides alkylthio and arylthio groups; and the counter ion Z is an anion selected from the group consisting of PF₆, BF₄, Cl, Br, CF₃CO₂, and CF₃SO₃. In one embodiment, R″ is selected from the group consisting of H, Cl, Br, CN, alkylthio and arylthio groups.

In another embodiment, the present disclosure provides thiopyrylium dyes and selenopyrylium dyes of general structure IV as SERS reporters attached to nanoparticles, for example, those comprised of gold (e.g. hollow gold nanoshells), silver, copper or combinations thereof, and methods of using these compositions for SERS and/or SERSS spectroscopy with incident light from 1000 nm to 1300 nm where E and E′ are independently selected from the chalcogen atoms S and Se; Ar, Ar′, Ar″, and Ar′″ are independently selected from the group consisting of phenyl (substituted or unsubstituted), 2-thienyl (substituted or unsubstituted), 3-thienyl (substituted or unsubstituted), 2-selenophenyl (substituted or unsubstituted), and 3-selenophenyl (substituted or unsubstituted); R is H; all R′s are H or together can form a five- or six-membered ring; R″ is selected from H, halides, pseudohalides, alkylthio and arylthio groups; and the counter ion Z is an anion selected from the group consisting of PF₆, BF₄, Cl, Br, CF₃CO₂, and CF₃SO₃. In one embodiment, R″ is selected from the group consisting of H, Cl, Br, CN, alkylthio and arylthio groups.

In another embodiment, the present disclosure provides thiopyrylium dyes and selenopyrylium dyes of general structure IV as SERS reporters attached to nanoparticles, for example, those comprised of gold (e.g. hollow gold nanoshells), silver, copper or combinations thereof, and methods of using these compositions for SERS and/or SERSS spectroscopy with incident light from a 1280-nm laser wherein E and E′ are independently selected from the chalcogen atoms S and Se; Ar, Ar′, Ar″, and Ar′″ are independently selected from the group consisting of phenyl (substituted or unsubstituted), 2-thienyl (substituted or unsubstituted), 3-thienyl (substituted or unsubstituted), 2-selenophenyl (substituted or unsubstituted), and 3-selenophenyl (substituted or unsubstituted); R is H; all R′s are H or together can form a five- or six-membered ring; R″ is selected from H, halides or pseudohalides, alkylthio and arylthio groups; and the counter ion Z is an anion selected from the group consisting of PF₆, BF₄, Cl, Br, CF₃CO₂, and CF₃SO₃. In one embodiment, R″ is selected from the group consisting of H, Cl, Br, CN, alkylthio and arylthio groups.

In another embodiment, the present disclosure provides thiopyrylium dyes and selenopyrylium dyes of general structure IV as SERS reporters attached to nanoparticles, for example, those comprised of gold (e.g. hollow gold nanoshells), silver, copper or combinations thereof, and methods of using these compositions for SERS and/or SERSS spectroscopy with incident light from 1000 nm to 1300 nm wherein E and E′ are independently selected from the chalcogen atoms S and Se; Ar, Ar′, Ar″, and Ar′″ are independently selected from the group consisting of phenyl (substituted or unsubstituted), 2-thienyl (substituted or unsubstituted), and 2-selenophenyl (substituted or unsubstituted); R is H; all R′s are H or together can form a five- or six-membered ring; R″ is selected from the group consisting of H, halides, pseudohalides alkylthio and arylthio groups; and the counter ion Z is an anion selected from the group consisting of PF₆, BF₄, Cl, Br, CF₃CO₂, and CF₃SO₃. In one embodiment, R″ is selected from the group consisting of H, Cl, Br, CN, alkylthio and arylthio groups. In another embodiment, the present disclosure provides thiopyrylium dyes and selenopyrylium dyes of general structure IV as SERS reporters attached to nanoparticles, for example, those comprised of gold (e.g. hollow gold nanoshells), silver, copper or combinations thereof, and methods of using these compositions for SERS and/or SERSS spectroscopy with incident light from a 1280-nm laser wherein E and E′ are independently selected from the chalcogen atoms S and Se; Ar, Ar′, Ar″, and Ar′″ are independently selected from the group consisting of phenyl (substituted or unsubstituted), 2-thienyl (substituted or unsubstituted), and 2-selenophenyl (substituted or unsubstituted); R is H; all R′s are H or together can form a five- or six-membered ring; R″ is selected from H, halides, pseudohalides alkylthio and arylthio groups; and the counter ion Z is an anion selected from the group consisting of PF₆, BF₄, Cl, Br, CF₃CO₂, and CF₃SO₃. In one embodiment, R″ is selected from the group consisting of H, Cl, Br, CN, alkylthio and arylthio groups.

In another embodiment, the present disclosure provides thiopyrylium dyes and selenopyrylium dyes of general structure IV as SERS reporters attached to nanoparticles, for example, those comprised of gold (e.g. hollow gold nanoshells), silver, copper or combinations thereof, and methods of using these compositions for SERS and/or SERSS spectroscopy with incident light from 1000 nm to 1300 nm wherein E and E′ are independently selected from the chalcogen atoms S and Se; Ar, Ar′, Ar″, and Ar′″ are independently selected from the group consisting of phenyl (substituted or unsubstituted), 2-thienyl (substituted or unsubstituted), and 2-selenophenyl (substituted or unsubstituted); R is H; all R′s together form a six-membered ring, R″ is Cl; and the counter ion Z is an anion selected from the group consisting of PF₆, BF₄, Cl, Br, CF₃CO₂, and CF₃SO₃.

In another embodiment, the present disclosure provides thiopyrylium dyes and selenopyrylium dyes of general structure IV as SERS reporters attached to nanoparticles, for example, those comprised of gold (e.g. hollow gold nanoshells), silver, copper or combinations thereof, and methods of using these compositions for SERS and/or SERSS spectroscopy with incident light from a 1280-nm laser wherein E and E′ are independently selected from the chalcogen atoms S and Se; Ar, Ar′, Ar″, and Ar′″ are independently selected from the group consisting of phenyl (substituted or unsubstituted), 2-thienyl (substituted or unsubstituted), and 2-selenophenyl (substituted or unsubstituted); R is H; all R′s together form a six-membered ring; R″ is Cl; and the counter ion Z is an anion selected from the group consisting of PF₆, BF₄, Cl, Br, CF₃CO₂, and CF₃SO₃.

In another embodiment, the present disclosure provides thiopyrylium dyes and selenopyrylium dyes of general structure IV wherein E and E′ are independently selected from the chalcogen atoms S and Se; Ar, Ar′, Ar″, and Ar′″ are independently selected from the group consisting of phenyl, 2-thienyl, and 2-selenophenyl wherein at least two of the groups Ar, Ar′, Ar″, or Ar′″ are 2-thienyl or 2-selenophenyl; R is H; all R′s together form a six-membered ring; R″ is Cl; and the counter ion Z is PF₆, which are novel compositions of matter. The subject disclosure also provides thiopyrylium dyes and selenopyrylium dyes of general structure IV as SERS reporters attached to, for example, hollow gold, silver or copper nanoparticles, and methods of using these compositions for SERS and/or SERSS spectroscopy with incident light from 1000 nm to 1300 nm wherein E and E′ are independently selected from the chalcogen atoms S and Se; Ar, Ar′, Ar″, and Ar′″ are independently selected from the group consisting of phenyl, 2-thienyl, and 2-selenophenyl wherein at least two of the groups Ar, Ar′, Ar″, or Ar′″ are 2-thienyl or 2-selenophenyl; R is H; all R′s together form a six-membered ring; R″ is Cl; and the counter ion Z is PF₆. In other embodiments of the composition and method, the incident light is from a 1280-nm laser.

In another embodiment, the present disclosure provides a thiopyrylium dye of general structure I wherein E=E′=S, Ar═Ar′═Ar″═Ar′″=2-thienyl, R═H, and Z═PF₆.

In another embodiment, the present disclosure provides a thiopyrylium dye of general structure I wherein E=E′=S, Ar═Ar′=Ph, Ar″=Ar′″=2-thienyl, R═H, and Z═PF₆.

In another embodiment, the present disclosure provides a selenopyrylium dye of general structure I wherein E=Se, E′=S, Ar═Ar′═Ar″═Ar′″=2-thienyl, R═H, and Z═PF₆.

In another embodiment, the present disclosure provides a selenopyrylium dye of general structure I wherein E=Se, E′=S, Ar═Ar′=Ph, Ar″=Ar′″=2-thienyl, R═H, and Z═PF6.

In another embodiment, the present disclosure provides a selenopyrylium dye of general structure I wherein E=Se, E′=S, Ar═Ar′=2-thienyl, Ar″=Ar′″=Ph, R═H, and Z═PF₆.

In another embodiment, the present disclosure provides a selenopyrylium dye of general structure I wherein E=E′=Se, Ar═Ar′═Ar″═Ar′″=2-thienyl, R═H, and Z═PF₆.

In another embodiment, the present disclosure provides a selenopyrylium dye of general structure I wherein E=E′=Se, Ar═Ar′=Ph, Ar″=Ar′″=2-thienyl, R═H, and Z═PF₆.

In another embodiment, the present disclosure provides a thiopyrylium dye of general structure I wherein E=E′=S, Ar═Ar′═Ar″═Ar′″=2-selenophenyl, R═H, and Z═PF₆.

In another embodiment, the present disclosure provides a thiopyrylium dye of general structure I wherein E=E′=S, Ar═Ar′=Ph, Ar″=Ar′″=2-selenophenyl, R═H, and Z═PF₆.

In another embodiment, the present disclosure provides a selenopyrylium dye of general structure I wherein E=Se, E′=S, Ar═Ar′═Ar″═Ar′″=2-selenophenyl, R═H, and Z═PF₆.

In another embodiment, the present disclosure provides a selenopyrylium dye of general structure I wherein E=Se, E′=S, Ar═Ar′=2-thienyl, Ar″=Ar′″=2-selenophenyl, R═H, and Z═PF₆.

In another embodiment, the present disclosure provides a selenopyrylium dye of general structure I wherein E=Se, E′=S, Ar═Ar′=2-selenophenyl, Ar″=Ar′″=2-thienyl, R═H; and Z═PF₆.

In another embodiment, the present disclosure provides a selenopyrylium dye of general structure I wherein E=E′=Se, Ar═Ar′=2-thienyl, Ar″=Ar′″=2-selenophenyl, R═H; and Z═PF₆.

In another embodiment, the present disclosure provides thiopyrylium dye of general structure I wherein E=E′=S, Ar═Ar′═Ar″═Ar′″=2-selenophenyl, R═H, and Z═PF₆.

In another embodiment, the present disclosure provides a thiopyrylium dye of general structure II wherein E=E′=S, Ar═Ar′═Ar″═Ar′″=2-thienyl, R═R′═R″=H, and Z═PF₆.

In another embodiment, the present disclosure provides a thiopyrylium dye of general structure II wherein E=E′=S, Ar═Ar′=Ph, Ar″=Ar′″=2-thienyl, R═R′═R″=H, and Z═PF₆.

In another embodiment, the present disclosure provides a selenopyrylium dye of general structure II wherein E=Se, E′=S, Ar═Ar′═Ar″═Ar′″=2-thienyl, R═R′═R″=H, and Z═PF₆.

In another embodiment, the present disclosure provides a selenopyrylium dye of general structure II wherein E=Se, E′=S, Ar═Ar′=Ph, Ar″=Ar′″=2-thienyl, R═R′ ═R″=H, and Z═PF₆.

In another embodiment, the present disclosure provides a selenopyrylium dye of general structure II wherein E=Se, E′=S, Ar═Ar′=2-thienyl, Ar″=Ar′″=Ph, R═R′ ═R″=H, and Z═PF₆.

In another embodiment, the present disclosure provides a selenopyrylium dye of general structure II wherein E=E′=Se, Ar═Ar′═Ar″═Ar′″=2-thienyl, R═R′═R″=H, and Z═PF₆.

In another embodiment, the present disclosure provides a selenopyrylium dye of general structure II wherein E=E′=Se, Ar═Ar′=Ph, Ar″=Ar′″=2-thienyl, R═R′═R″=H, and Z═PF₆.

In another embodiment, the present disclosure provides a thiopyrylium dye of general structure II wherein E=E′=S, Ar═Ar′═Ar″═Ar′″=2-selenophenyl, R═R′═R″ ═H, and Z═PF₆.

In another embodiment, the present disclosure provides a thiopyrylium dye of general structure II wherein E=E′=S, Ar═Ar′=Ph, Ar″=Ar′″=2-selenophenyl, R═R′ ═R″=H, and Z═PF₆.

In another embodiment, the present disclosure provides a selenopyrylium dye of general structure II wherein E=Se, E′=S, Ar═Ar′═Ar″═Ar′″=2-selenophenyl, R═R′═R″=H, and Z═PF₆.

In another embodiment, the present disclosure provides a selenopyrylium dye of general structure II wherein E=Se, E′=S, Ar═Ar′=2-thienyl, Ar″=Ar′″=2-selenophenyl, R═R′═R″=H, and Z═PF₆.

In another embodiment, the present disclosure provides a selenopyrylium dye of general structure II wherein E=Se, E′=S, Ar═Ar′=2-selenophenyl, Ar″=Ar′″=2-thienyl, R═R′═R″=H, and Z═PF₆.

In another embodiment, the present disclosure provides a selenopyrylium dye of general structure II wherein E=E′=Se, Ar═Ar′=2-thienyl, Ar″=Ar′″=2-selenophenyl, R═R′═R″=H, and Z═PF₆.

In another embodiment, the present disclosure provides a thiopyrylium dye of general structure II wherein E=E′=S, Ar═Ar′═Ar″═Ar′″=2-selenophenyl, R═R′═R″ ═H, and Z═PF₆.

In another embodiment, the present disclosure provides a thiopyrylium dye of general structure III wherein E=E′=S, Ar═Ar′═Ar″═Ar′″=2-thienyl, R═H, and Z=PF₆.

In another embodiment, the present disclosure provides a thiopyrylium dye of general structure III wherein E=E′=S, Ar═Ar′=Ph, Ar″=Ar′″=2-thienyl, R═H, and Z ═PF₆.

In another embodiment, the present disclosure provides a selenopyrylium dye of general structure III wherein E=Se, E′=S, Ar═Ar′═Ar″═Ar′″=2-thienyl, R═H, and Z═PF₆.

In another embodiment, the present disclosure provides a selenopyrylium dye of general structure III wherein E=Se, E′=S, Ar═Ar′=Ph, Ar″=Ar′″=2-thienyl, R═H, and Z═PF₆.

In another embodiment, the present disclosure provides a selenopyrylium dye of general structure III wherein E=Se, E′=S, Ar═Ar′=2-thienyl, Ar″=Ar′″=Ph, R═H, and Z═PF₆.

In another embodiment, the present disclosure provides a selenopyrylium dye of general structure III wherein E=E′=Se, Ar═Ar′═Ar″═Ar′″=2-thienyl, R═H, and Z PF₆.

In another embodiment, the present disclosure provides a selenopyrylium dye of general structure III wherein E=E′=Se, Ar═Ar′=Ph, Ar″=Ar′″=2-thienyl, R═H, and Z═PF₆.

In another embodiment, the present disclosure provides a thiopyrylium dye of general structure III wherein E=E′=S, Ar═Ar′═Ar″═Ar′″=2-selenophenyl, R═H, and Z═PF₆.

In another embodiment, the present disclosure provides a thiopyrylium dye of general structure III wherein E=E′=S, Ar═Ar′=Ph, Ar″=Ar′″=2-selenophenyl, R═H, and Z═PF₆.

In another embodiment, the present disclosure provides a selenopyrylium dye of general structure III wherein E=Se, E′=S, Ar═Ar′═Ar″═Ar′″=2-selenophenyl, R═H, and Z═PF₆.

In another embodiment, the present disclosure provides a selenopyrylium dye of general structure III wherein E=Se, E′=S, Ar═Ar′=2-thienyl, Ar″=Ar′″=2-selenophenyl, R═H, and Z═PF₆.

In another embodiment, the present disclosure provides a selenopyrylium dye of general structure III wherein E=Se, E′=S, Ar═Ar′=2-selenophenyl, Ar″=Ar′″=2-thienyl, and Z═PF₆.

In another embodiment, the present disclosure provides a selenopyrylium dye of general structure III wherein E=E′=Se, Ar═Ar′=2-thienyl, Ar″=Ar′″=2-selenophenyl, and Z═PF₆.

In another embodiment, the present disclosure provides a thiopyrylium dye of general structure III wherein E=E′=S, Ar═Ar′═Ar″═Ar′″=2-selenophenyl, R═H, and Z═PF₆.

In another embodiment, the present disclosure provides a thiopyrylium dye of general structure IV wherein E=E′=S, Ar═Ar′═Ar″═Ar′″=2-thienyl, R═H; R′, all R′s together form a six-membered ring and R″=Cl, and Z═PF₆.

In another embodiment, the present disclosure provides a selenopyrylium dye of general structure IV wherein E=E′=Se, Ar═Ar′═Ar″═Ar′″=2-thienyl, R═H; all R′s together form a six-membered ring and R″=Cl, and Z═PF₆.

In another embodiment, the present disclosure provides a thiopyrylium dye of general structure IV wherein E=E′=S, Ar═Ar′═Ar″═Ar′″=2-selenophenyl, R═H; R′, all R′s together form a six-membered ring and R″=Cl, and Z═PF₆.

In another embodiment, the present disclosure provides a selenopyrylium dye of general structure IV wherein E=E′=Se, Ar═Ar′═Ar″═Ar′″=2-selenophenyl, R═H; R′, all R′s together form a six-membered ring and R″=Cl, and Z═PF₆.

In another embodiment, the present disclosure provides a thiopyrylium dye of general structure V wherein E=E′=S, Ar′═Ar″═Ar′″=2-thienyl, R═H, and Z═PF₆.

In another embodiment, the present disclosure provides a thiopyrylium dye of general structure V wherein E=E′=S, Ar′=Ph, Ar″=Ar′″=2-thienyl, R═H, and Z═PF₆.

In another embodiment, the present disclosure provides a selenopyrylium dye of general structure V wherein E=Se, E′=S, Ar′═Ar″═Ar′″=2-thienyl, R═H, and Z═PF₆.

In another embodiment, the present disclosure provides a selenopyrylium dye of general structure V wherein E=Se, E′=S, Ar′=Ph, Ar″=Ar′″=2-thienyl, R═H, and Z ═PF₆.

In another embodiment, the present disclosure provides a selenopyrylium dye of general structure V wherein E=Se, E′=S, Ar′=2-thienyl, Ar″=Ar′″=Ph, R═H, and Z PF₆.

In another embodiment, the present disclosure provides a selenopyrylium dye of general structure V wherein E=E′=Se, Ar′═Ar″═Ar′″=2-thienyl, R═H, and Z═PF₆.

In another embodiment, the present disclosure provides a selenopyrylium dye of general structure V wherein E=E′=Se, Ar′=Ph, Ar″=Ar′″=2-thienyl, R═H, and Z═PF₆.

In another embodiment, the present disclosure provides a thiopyrylium dye of general structure V wherein E=E′=S, Ar′═Ar″═Ar′″=2-selenophenyl, R═H, and Z═PF₆.

In another embodiment, the present disclosure provides a thiopyrylium dye of general structure V wherein E=E′=S, Ar′=Ph, Ar″=Ar′″=2-selenophenyl, R═H, and Z ═PF₆.

In another embodiment, the present disclosure provides a selenopyrylium dye of general structure V wherein E=Se, E′=S, Ar′═Ar″═Ar′″=2-selenophenyl, R═H, and Z═PF₆.

In another embodiment, the present disclosure provides a selenopyrylium dye of general structure V wherein E=Se, E′=S, Ar′=2-thienyl, Ar″=Ar′″=2-selenophenyl, R═H, and Z═PF₆.

In another embodiment, the present disclosure provides a selenopyrylium dye of general structure V wherein E=Se, E′=S, Ar′=2-selenophenyl, Ar″=Ar′″=2-thienyl, and Z═PF₆.

In another embodiment, the present disclosure provides a selenopyrylium dye of general structure V wherein E=E′=Se, Ar′=2-thienyl, Ar″=Ar′″=2-selenophenyl, and Z═PF₆.

In another embodiment, the present disclosure provides a thiopyrylium dye of general structure V wherein E=E′=S, Ar′═Ar″═Ar′″=2-selenophenyl, R═H, and Z═PF₆.

In another embodiment, the present disclosure provides a thiopyrylium dye of general structure VI or VII wherein E=E′=S, Ar′═Ar″=2-thienyl, R═H, and Z═PF₆.

In another embodiment, the present disclosure provides a thiopyrylium dye of general structure VI or VII wherein E=E′=S, Ar′=Ph, Ar′″=2-thienyl, R═H, and Z═PF₆.

In another embodiment, the present disclosure provides a selenopyrylium dye of general structure VI or VII wherein E=Se, E′=S, Ar′═Ar′″=2-thienyl, R═H, and Z═PF₆.

In another embodiment, the present disclosure provides a selenopyrylium dye of general structure VI or VII wherein E=Se, E′=S, Ar′=Ph, Ar′″=2-thienyl, R═H, and Z═PF₆.

In another embodiment, the present disclosure provides a selenopyrylium dye of general structure VI or VII wherein E=Se, E′=S, Ar′=2-thienyl, Ar′″=Ph, R═H, and Z═PF₆.

In another embodiment, the present disclosure provides a selenopyrylium dye of general structure VI or VII wherein E=E′=Se, Ar′═Ar′″=2-thienyl, R═H, and Z═PF₆.

In another embodiment, the present disclosure provides a selenopyrylium dye of general structure VI or VII wherein E=E′=Se, Ar′=Ph,′″=2-thienyl, R═H, and Z═PF₆.

In another embodiment, the present disclosure provides a thiopyrylium dye of general structure VI or VII wherein E=E′=S, Ar′═Ar′″=2-selenophenyl, R═H, and Z═PF₆.

In another embodiment, the present disclosure provides a thiopyrylium dye of general structure VI or VII wherein E=E′=S, Ar′=Ph, Ar′″=2-selenophenyl, R═H, and Z═PF₆.

In another embodiment, the present disclosure provides a selenopyrylium dye of general structure VI or VII wherein E=Se, E′=S, Ar′═Ar′″=2-selenophenyl, R═H, and Z═PF₆.

In another embodiment, the present disclosure provides a selenopyrylium dye of general structure VI or VII wherein E=Se, E′=S, Ar′=2-thienyl, Ar′″=2-selenophenyl, R═H, and Z═PF₆.

In another embodiment, the present disclosure provides a selenopyrylium dye of general structure VI or VII wherein E=Se, E′=S, Ar′=2-selenophenyl, Ar′″=2-thienyl, and Z═PF₆.

In another embodiment, the present disclosure provides a selenopyrylium dye of general structure VI or VII wherein E=E′=Se, Ar′=2-thienyl, Ar′″=2-selenophenyl, and Z═PF₆.

In another embodiment, the present disclosure provides a thiopyrylium dye of general structure VI or VII wherein E=E′=S, Ar′═Ar′″=2-selenophenyl, R═H, and Z═PF₆.

In an object, the present disclosure provides methods of preparing a nanostructure. In an embodiment, a method of preparing a nanostructure comprises: introducing a core to a reporter molecule, where the reporter molecule bonds to the core and the reporter molecule is selected from the chalcogenopyrylium dyes described herein; and optionally, disposing an encapsulating material onto the core and reporter molecule (e.g., reacting a material to form an encapsulating material), where the reporter molecule has a measurable surface-enhanced Raman spectroscopic signature. If applicable, the encapsulating material can be, for example, silica. Other suitable encapsulating materials include silica-based materials such as xerogels from tetraalkoxy silanes or organically modified xerogels from organotrialkoxy silanes and tetraalkoxy silanes; also polyethylene glycol (PEG) such as PEG 5000; and organic polymers such as, but not limited to, polyvinylethylene (PVE).

The method may further comprise conjugating (e.g., covalently or non-covalently bonding) one or more targeting moieties (which can be part of a probe molecule or probe molecules) directly to a surface of the core or to a surface of the core via a linking group. A targeting moiety is any moiety that specifically interacts with (e.g., binds) a target molecule. A probe molecule can comprise a targeting moiety. Examples of targeting moieties (e.g., probe molecules) include, but are not limited to, antibodies, aptamers, synthetic receptors, DNA sequences, proteins, peptides, and the like. Examples of suitable conjugation methods and linkers are known in the art.

In an object, the present disclosure provides uses of the composite nanostructures. The composite nanostructures can be used in methods such as, for example, anti-counterfeit methods, covert tagging methods, cytometry methods (e.g., a flow cytometry system), chemical array methods, biomolecule array methods, biosensing methods, bioimaging methods, biolabeling methods, high-speed screening methods, gene expression methods, protein expression methods, medical diagnostic methods, diagnostic methods, and microfluidic methods.

One embodiment of an exemplary method of detecting a target molecule, among others, includes: attaching a target molecule to a nanostructure as described above; exciting the reporter molecule with a source of radiation; and measuring the surface-enhanced Raman spectroscopy spectrum of the nanostructure corresponding to the reporter molecule in order to determine the presence of the target molecule.

The present disclosure provides a method of detecting one or more target molecules in a sample. The method includes attaching a target molecule (e.g., via interaction with) a probe molecule (i.e., a molecule having a targeting moiety) to the nanostructure and measuring the SERS spectrum of the nanostructure, where the detection of SERS spectrum specific for the reporter molecule indicates the presence of the target molecule specific for the probe molecule (i.e., a molecule having a targeting moiety). The SERS active composite nanostructure can be used to detect the presence of one or more target molecules in chemical array systems, bioimaging and biomolecular array systems. In addition, SERS active composite nanostructures can be used to enhance encoding and multiplexing capabilities in various types of systems.

For example, a method for detecting one or more target molecules in a sample comprises: contacting (e.g., administering to) an individual or other biological material, such as, for example, plants, bacteria, viruses, and other organisms, or a portion thereof, with one or more of the composite nanostructures of the present disclosure, and obtaining surface-enhanced Raman spectroscopy data (e.g., a surface-enhanced Raman spectrum) of a portion of the individual after contact of the portion of the individual with the one or more said composite nanostructures, where observation of surface-enhanced Raman spectroscopy data attributable (e.g., specifically attributable) to a particular composite nanostructure of the one or more said composite nanostructures indicates the presence of the target molecule in the portion of the individual corresponding to the targeting moiety of the particular nanostructure. The method may further comprises obtaining surface-enhanced Raman spectroscopy data (e.g., a surface-enhanced Raman spectrum) of one or more additional portions of the individual after contact of the one or more additional portions of the individual with the one or more said composite nanostructures. The method may further comprise generating an image of at least a portion of the individual using the surface-enhanced Raman spectroscopy data from the portion and, optionally, additional portions of the individual.

An individual may be a human or non-human animal. An individual can be contacted with (e.g., administered) composite nanostructures by methods known in the art. The composite nanostructures can be administered systemically (e.g., by intravenous delivery) or locally to a desired area of an individual. The composite nanostructures are contacted (e.g., administered) prior to obtaining surface-enhanced Raman spectroscopy data from a portion of the individual or other biological material. Composite nanostructures can accumulate in a specific portion (e.g., a specific tissue) of the individual or other biological material as a result of the targeting moiety binding to a target molecule.

Surface-enhanced Raman spectroscopy data (e.g., a surface-enhanced Raman spectrum) can be obtained by methods known in the art. For example, surface-enhanced Raman spectroscopy data (e.g., a surface-enhanced Raman spectrum) is obtained using a laser having a wavelength of 780 nm to 1600 nm, including all nm values and ranges therebetween. In another example, surface-enhanced Raman spectroscopy data (e.g., a surface-enhanced Raman spectrum) is obtained using a laser having a wavelength of 1000 nm to 1600 nm (e.g., 1064 nm or 1280 nm).

In one embodiment, a flow cytometer can be used in multiplexed assay procedures for detecting one or more target molecules using one or more SERS active composite nanostructure. Flow cytometry is an optical technique that analyzes particular particles (e.g., SERS active composite nanostructures) in a fluid mixture based on the particles' optical characteristics. Flow cytometers hydrodynamically focus a fluid suspension of SERS active composite nanostructures into a thin stream so that the SERS active composite nanostructures flow down the stream in substantially single file and pass through an examination zone. A focused light beam, such as a laser beam, illuminates the SERS active composite nanostructures as they flow through the examination zone. Optical detectors within the flow cytometer measure certain characteristics of the light as it interacts with the SERS active composite nanostructures. Commonly used flow cytometers can measure SERS active composite nanostructure emission at one or more wavelengths.

For example, a flow cytometry method comprises, subjecting a plurality of cells to flow cytometry, where the cells comprise composite nanostructures of the present disclosure; obtaining surface-enhanced Raman spectroscopy data (e.g., a surface-enhanced Raman spectrum) for individual cells; and separating the cells based the surface-enhanced Raman spectroscopy data (e.g., a surface-enhanced Raman spectrum) obtained for the individual cells.

One or more target molecules can be detected using a SERS active composite nanostructures and one or more probes having an affinity for one or more of the target molecules. Each SERS active composite nanostructure has a reporter molecule that corresponds to the probe. Prior to being introduced to the flow cytometer, the SERS active composite nanostructures specific for certain target molecules are mixed with a sample that may include one or more target molecules. The SERS active composite nanostructures interact with (e.g., bond or hybridize) the corresponding target molecules for which the probe has an affinity.

Next, the SERS active composite nanostructures are introduced to the flow cytometer. As discussed above, the flow cytometer is capable of detecting the SERS active composite nanostructure after exposure to a first energy. Detection of a certain Raman spectrum corresponding to a certain reporter molecule indicates that a target molecule is present in the sample.

Step(s) of the methods disclosed herein are sufficient to produce the compounds, composite nanostructures, or methods of using the compounds and/or composite nanostructures of the present disclosure. Thus, in various examples, any such method consists essentially of a combination of one or more of the steps of the methods disclosed herein. In various other examples, any such method consists of such step(s).

The following examples are presented to illustrate the present disclosure. They are not intended to be limiting in any manner.

### Example 1

Here, we describe the design of a small library of thiophene- and selenophene-substituted chalcogenopyrylium dyes 1-14 (Scheme 1) that are sensitive SERS reporters on hollow gold nanoshells operating with 1064-nm and 1280-nm excitation. The chalcogenopyrylium dyes allow fine tuning of wavelengths of absorption through the choice of chalcogen atoms in the pyrylium/pyranyl rings and the substituents at the 2- and 6-positions of these rings. Since the SERS effect decreases exponentially as a function of distance from the nanoparticle, it is important that the Raman reporter be near the Au surface. Due to this distance dependence, planar molecules capable of lying flat on the surface should experience the largest enhancement in Raman intensity. X-ray structural studies have shown that the chalcogenopyrylium/chalcogenopyranyl rings and methine carbon of chalcogenopyrylium dyes related to 1-8 are coplanar and computational studies predict similar coplanarity in chalcogenopyrylium trimethine dyes 9-14. Other structural and computational studies have shown that five-membered rings such as thiophene or selenophene can be coplanar with attached chalcogenopyrylium/chalcogenopyranyl rings. The affinity of the reporter for the surface of Au is another important consideration. Thiophenes and selenophenes are both capable of forming self-assembled monolayers on gold. Selenolates have also been shown to have greater affinity for gold than thiolates. Chalcogenopyrylium dyes 1-14 incorporate all these features. The dyes 1-14 incorporate S and Se atoms in the chalcogenopyrylium core to provide attachment to gold and the 2-thienyl and 2-selenophenyl groups provide novel attachment points to gold for Raman reporters.

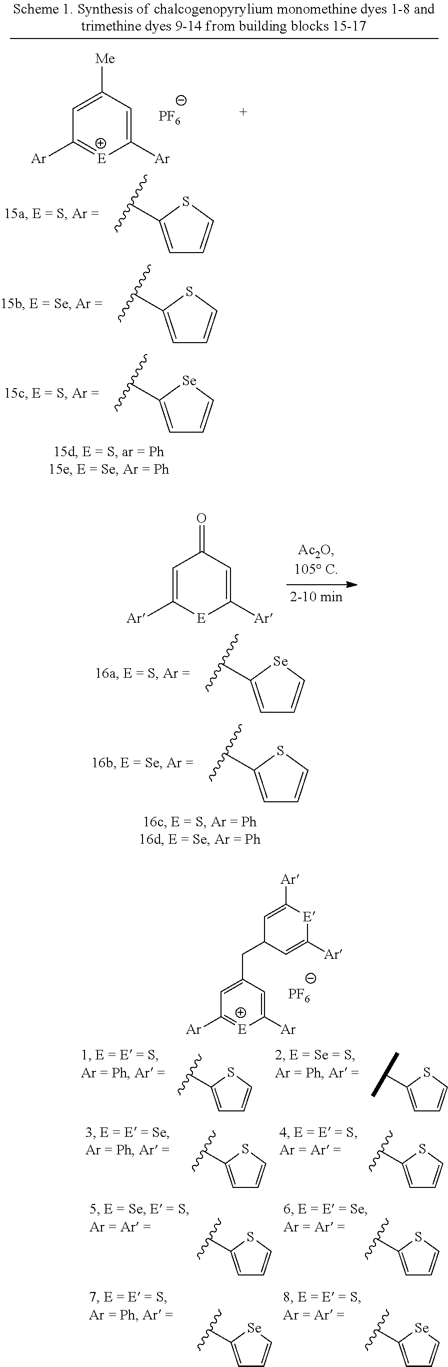

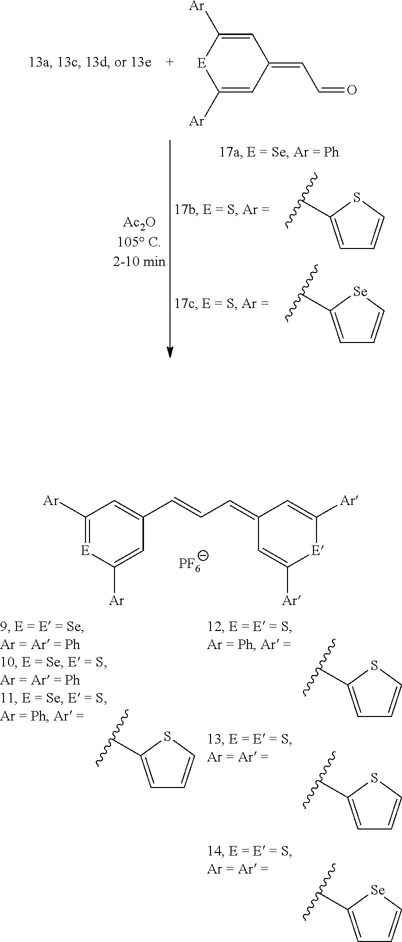

Results. Synthesis and Properties of the Chalcogenopyrylium Dyes. The synthesis of dyes 1-14 is summarized in Scheme 1. 4-Methylthiopyrylium and 4-methylselenopyrylium salts 15 were prepared by the addition of MeMgBr to the corresponding chalcogenopyranone 16 followed by treatment with aqueous HPF₆. Condensation of compound 15 either with the chalcogenopyranone 16 or the (chalcogenopyranyl)acetaldehyde 17 in acetic anhydride gave monomethine dyes 1-8 or trimethine dyes 9-14, respectively. Values of absorption maxima, λₘₐₓ, in CH₂Cl₂ for 1-8 varied from 653 nm for 1 to 724 nm for 6 and values of the molar extinction coefficient, ε, were in the range of 1.1×10⁵ to 1.5×10⁵ M⁻¹ cm⁻¹ (Table 1). For trimethine dyes 9-14, values of λₘₐₓ in CH₂Cl₂ varied from 784 nm for 10 to 826 nm for 14 while values of E were in the range of 2.0×10⁵ to 2.8×10⁵ M⁻¹ cm⁻¹ (Table 1). The interchange of S and Se atoms in the chalcogenopyrylium backbone, the use of monomethine and trimethine bridges, and the interchange of phenyl, 2-thienyl, and 2-selenophenyl substituents at the 2-,2′-, 6-, and 6′-positions allow each dye to have a unique Raman fingerprint.

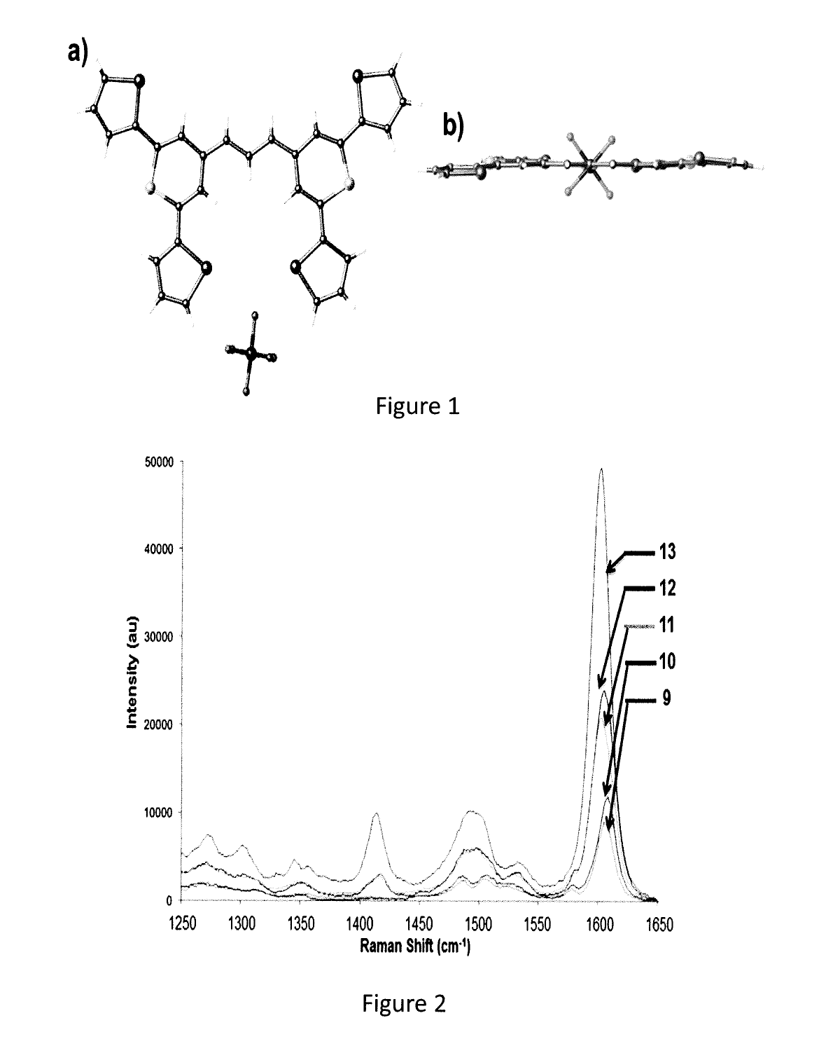

In order to demonstrate the unique structure of these dyes, crystals of dye 14 were grown from acetonitrile and the chemical structure was determined by X-ray crystallographic analysis. The results are shown in FIG. 1. Dye 14 has a transoid structure with the selenium atoms of the four selenophene substituents pointed away from the sulfur atoms of the thiopyrylium rings (FIG. 1a). If the structure is rotated 90° as shown in FIG. 1b, the coplanarity of the four selenophene rings with the pyrylium core is easily observed. The library of compounds 1-14 is uniquely designed to allow the closest approach of the SERS reporter to the noble metal surface. The S and Se atoms of the thiopyrylium/selenopyrylium rings allow attachment to the noble metal surface with the thiophene and selenophene substituents providing novel additional points of attachment to the noble metal surface.

The importance of the thiophene and selenophene rings to provide an enhanced SERS spectrum is shown in FIG. 2. Here, dyes 9-13 on gold nanoparticles (prepared by the addition of 7.5 ml 1% (w/v) sodium citrate to 1.0 L boiling 0.25 mM HAuCl₄) were excited with a 785-nm laser with 2-s acquisition time to give the spectra shown in FIG. 2. It is known that SERS nanotags are responsive to the 785-nm excitation while there appear to be no documented examples of SERS nanotags responsive to 1280-nm excitation. The Raman spectra for the five dyes are remarkably similar. Dyes 9 and 10 with four phenyl substituents gave weaker Raman spectra than dyes 11 and 12 with two phenyl substituents and two 2-thienyl substituents. Dye 13 with four 2-thienyl substituents gave the most intense Raman spectrum. Thiophene and selenophene substituents are novel attachment groups for SERS reporters.

TABLE 1
Values of the absorption maximum (λₘₐₓ) and molar extinction coefficient (ε) for
chalcogenopyrylium dyes 1-14 and the isolated yields for the
dye-forming reaction in their synthesis.

| | | | | | λₘₐₓ (CH₂Cl₂), | ε (CH₂Cl₂), | |
|---|---|---|---|---|---|---|---|
| Dye | E | E′ | Ar | Ar′ | nm | M⁻¹ cm⁻¹ | % yield |
| Dye 1 | S | S | Ph | 2-thienyl | 653 | 1.3 × 10⁵ | 91 |
| Dye 2 | Se | S | Ph | 2-thienyl | 676 | 1.3 × 10⁵ | 91 |
| Dye 3 | Se | Se | Ph | 2-thienyl | 699 | 1.5 × 10⁵ | 44 |
| Dye 4 | S | S | 2-thienyl | 2-thienyl | 676 | 1.2 × 10⁵ | 97 |
| Dye 5 | Se | S | 2-thienyl | 2-thienyl | 698 | 1.1 × 10⁵ | 96 |
| Dye 6 | Se | Se | 2-thienyl | 2-thienyl | 724 | 1.3 × 10⁵ | 93 |
| Dye 7 | S | S | Ph | 2-selenophenyl | 659 | 1.4 × 10⁵ | 85 |
| Dye 8 | S | S | 2-selenophenyl | 2-selenophenyl | 687 | 1.1 × 10⁵ | 75 |
| Dye 9 | Se | Se | Ph | Ph | 806 | 2.5 × 10⁵ | 86 |
| Dye 10 | Se | S | Ph | Ph | 784 | 2.0 × 10⁵ | 86 |
| Dye 11 | Se | S | Ph | 2-thienyl | 810 | 2.5 × 10⁵ | 87 |
| Dye 12 | S | S | Ph | 2-thienyl | 789 | 2.2 × 10⁵ | 88 |
| Dye 13 | S | S | 2-thienyl | 2-thienyl | 813 | 2.8 × 10⁵ | 94 |
| Dye 14 | S | S | 2-selenophenyl | 2-selenophenyl | 826 | 2.3 × 10⁵ | 87 |
| Dye 15 | S | S | Ph, Benzo | Ph, Benzo | 789 | 1.5 × 10⁵ | 73 |
| Dye 16 | Se | S | Ph, Benzo | Ph, Ph | 748 | 6.1 × 10⁴ | 74 |
| Dye 17 | Se | Se | 2-thienyl, Benzo | 2-thienyl, Benzo | 786 | 7.8 × 10⁴ | 84 |
| Dye 18 | S | S | 2-thienyl | 2-thienyl | 943 | — | 48 |
| Dye 19 | Se | Se | 2-selenophenyl | 2-selenophenyl | 1001 | — | — |
| Dye 20 | S | S | Ph | Ph | 1042 | 1.0 × 10⁵ | 40 |
| Dye 21 | S | S | 2-thienyl | 2-thienyl | 1119 | — | — |

Examples of Dyes 1-14 as SERS Reporters on Hollow Gold Nanoshells (HGNs). Synthesis of HGNs for Use with 1064-nm Excitation. The HGN synthesis was carried out under inert conditions using a standard Schlenk line to prevent the cobalt nanoparticles from prematurely oxidizing. The method described was modified slightly from previous reports. In a typical synthesis, cobalt chloride hexahydrate (100 μL, 0.4M; Fisher Scientific, 99.99%) and trisodium citrate dihydrate (550 μL, 0.1 M; Sigma-Aldrich, >99%) were added into deionised water (100 mL) and degassed several times (10 mins vacuum and 15 mins argon). Sodium borohydride (1 mL, 0.1 M; Fisher Scientific, 99%) was injected into the solution and allowed to react for a further 20 minutes (under constant argon flow) until hydrogen evolution ceased, indicating complete hydrolysis of the reductant. The solution was degassed again (8 min vacuum and 10 min argon) before chloroauric acid trihydrate (33 mL, 248 μM; Fisher Scientific, ACS reagent grade) was injected. This mixture was allowed to react for an additional 10 minutes under argon with vigorous stirring. Before being exposed to air, were an obvious colour change from brown to green was observed. Finally, trisodium citrate (500 μL, 0.1 M) was added to stabilise the hollow gold nanoshell solution. Post synthesis, the HGN solution was concentrated through centrifugation (5000×g) and the precipitate was re-dispersed in trisodium citrate solution (2 mM) to give a final concentration of 2.14 nM. The HGNs had a localized surface plasmon resonance (SPR) at 690 nm.

Characterization and Use of HGNs with 1064-nm Excitation. Investigation into the SERS properties of the HGNs were carried out by mixing concentrated HGN solution (135 μL) with Raman reporter solution; namely dyes 1-14, BPE and AZPY (15 μL, 10 μM; synthesised in-house or purchased from Sigma-Aldrich) and potassium chloride (150 μL, 30 mM; Sigma-Aldrich). The Raman measurements were performed using a Real Time Analyzer FT-Raman spectrometer and a laser excitation wavelength of 1064 nm. All the measurements had a 5 second acquisition time and a laser power operating at 420 mW. Each sample was prepared in triplicate and 5 scans of each replicate were recorded. Furthermore, all the Raman spectra have been background corrected. For the SERS particle dilution study, the optimum conditions were used (as stated above) and deionised water was added to obtain subsequent concentrations, over the concentration range 1.3 nM to 1 pM. All other experimental conditions were kept the same as those stated above.

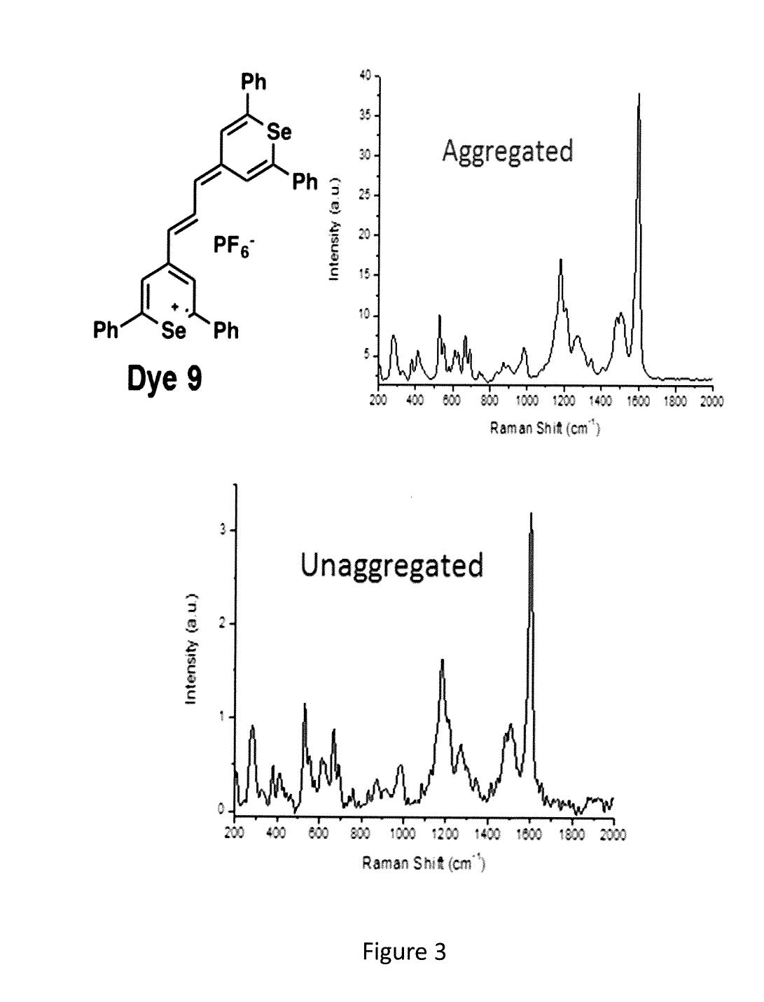

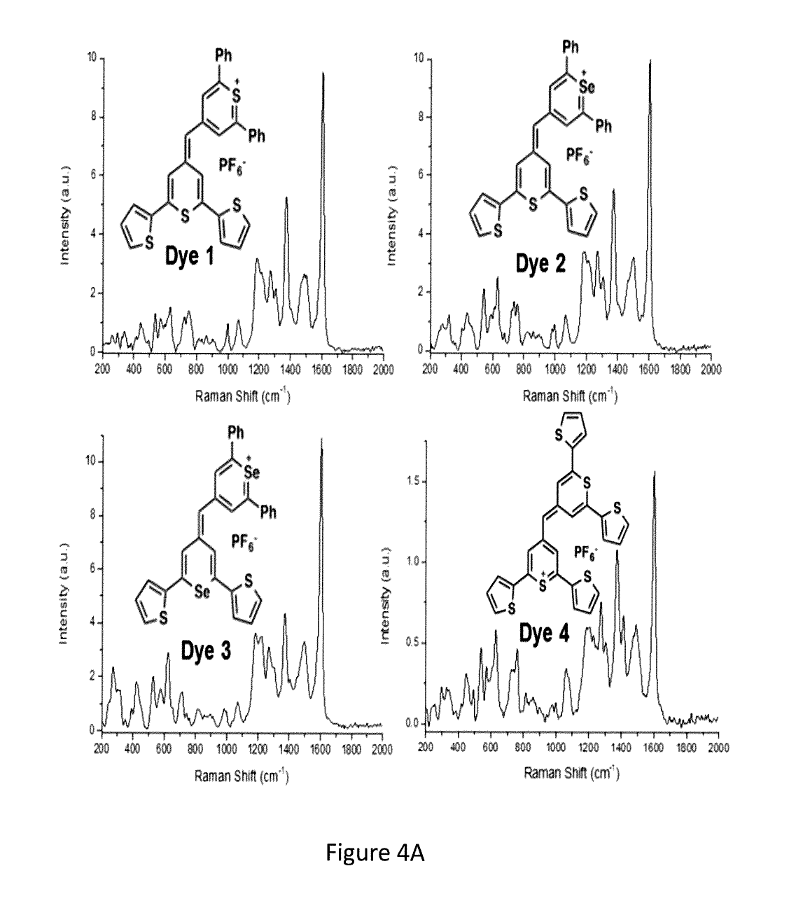

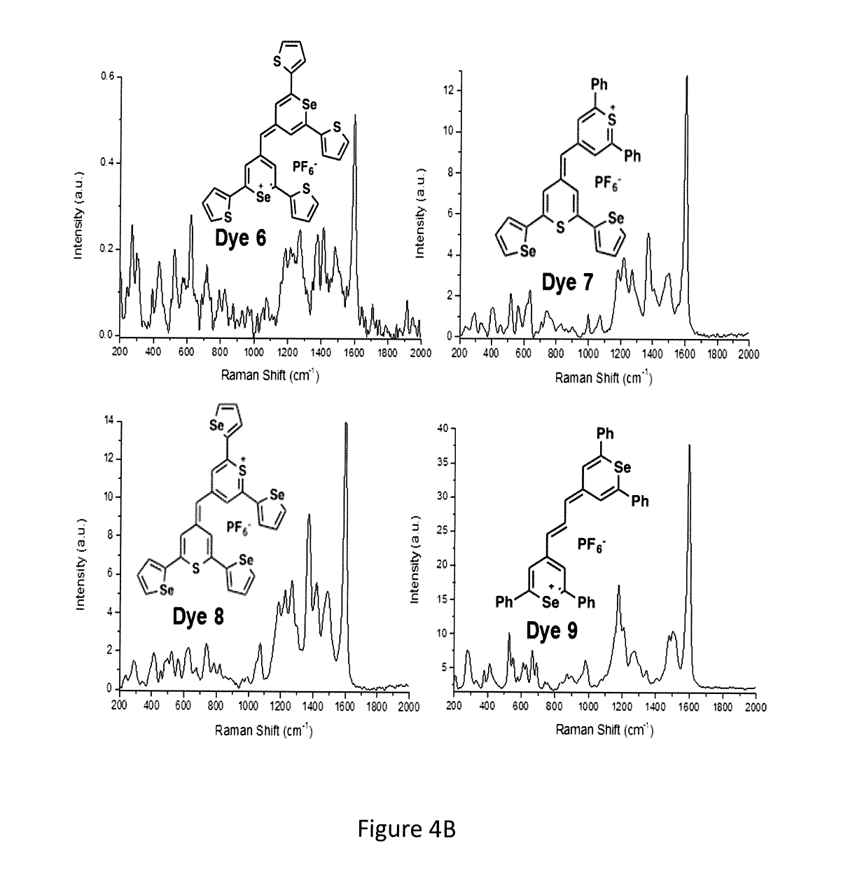

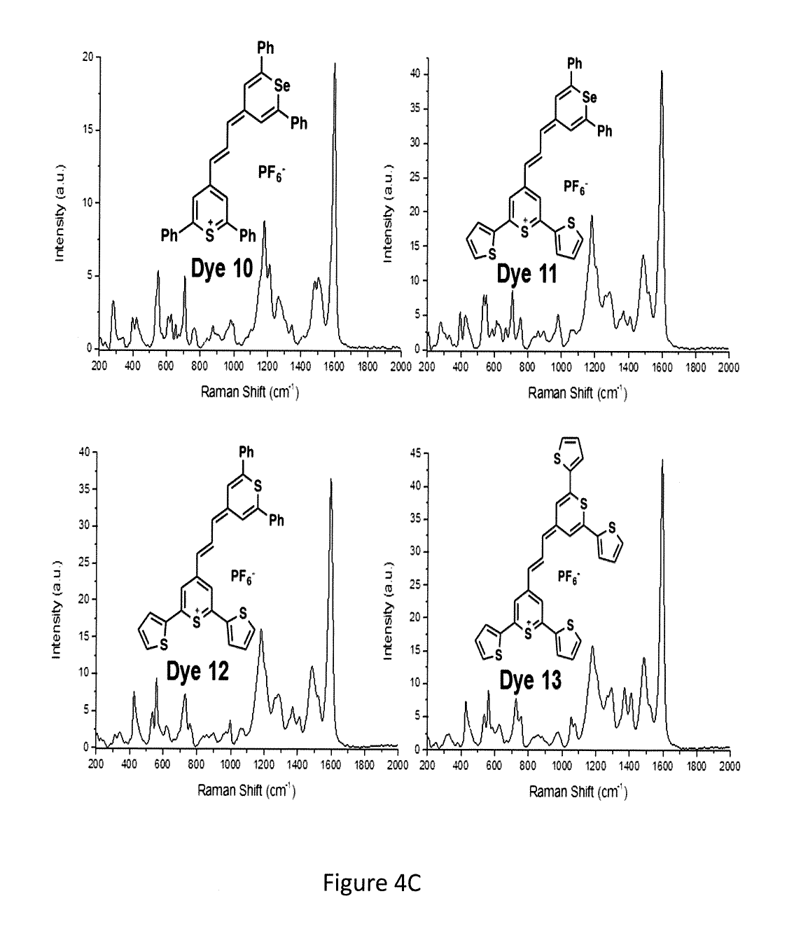

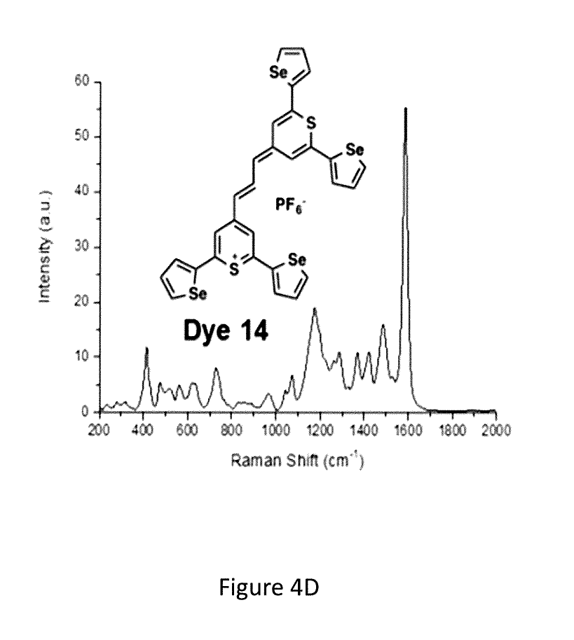

Examples of 1064-nm Excitation of Chalcogenopyrylium Dyes as SERS Reporters. A comparison of aggregated and unaggregated SERS spectra for the dye 9-HGN assemblies with 1064-nm excitation is shown in FIG. 3. The aggregated SERS spectra for the dye-HGN assemblies for dyes 1-14 with 1064 nm excitation is shown in FIG. 4.

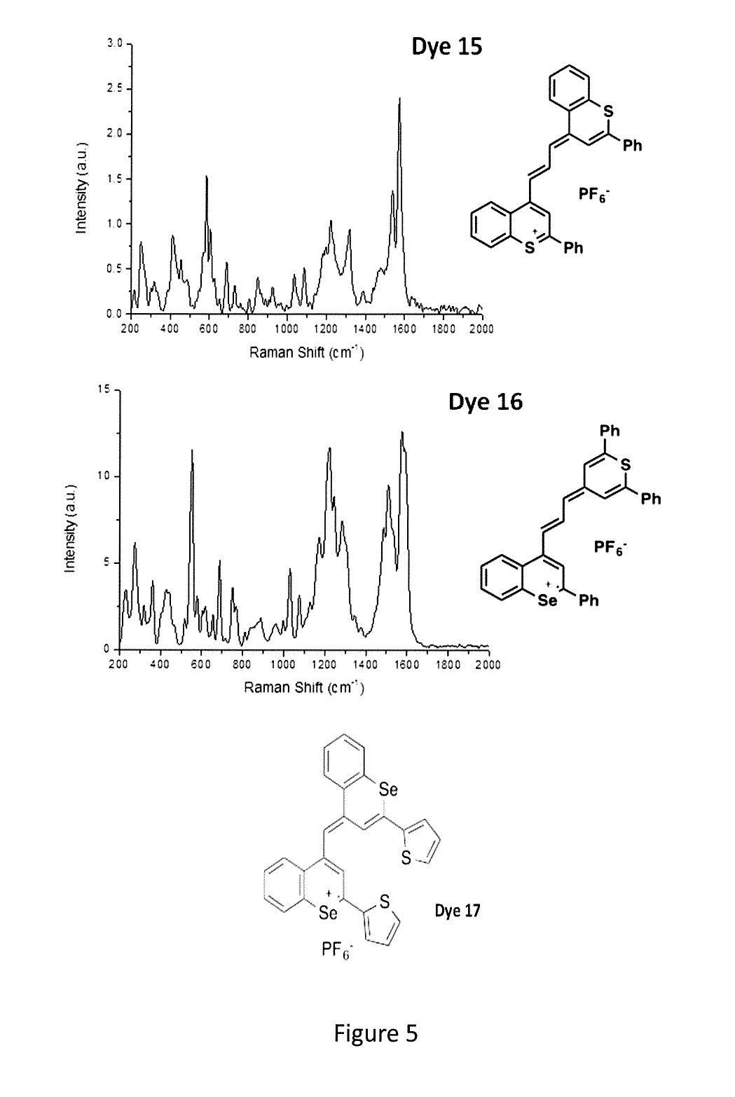

The benzo analogues of the chalcogenopyrylium dyes were also useful as SERS reporters on HGNs with 1064-nm excitation. As shown in FIG. 5, the dye 15-HGN and dye 16-HGN assemblies gave strong SERS signals when aggregated.

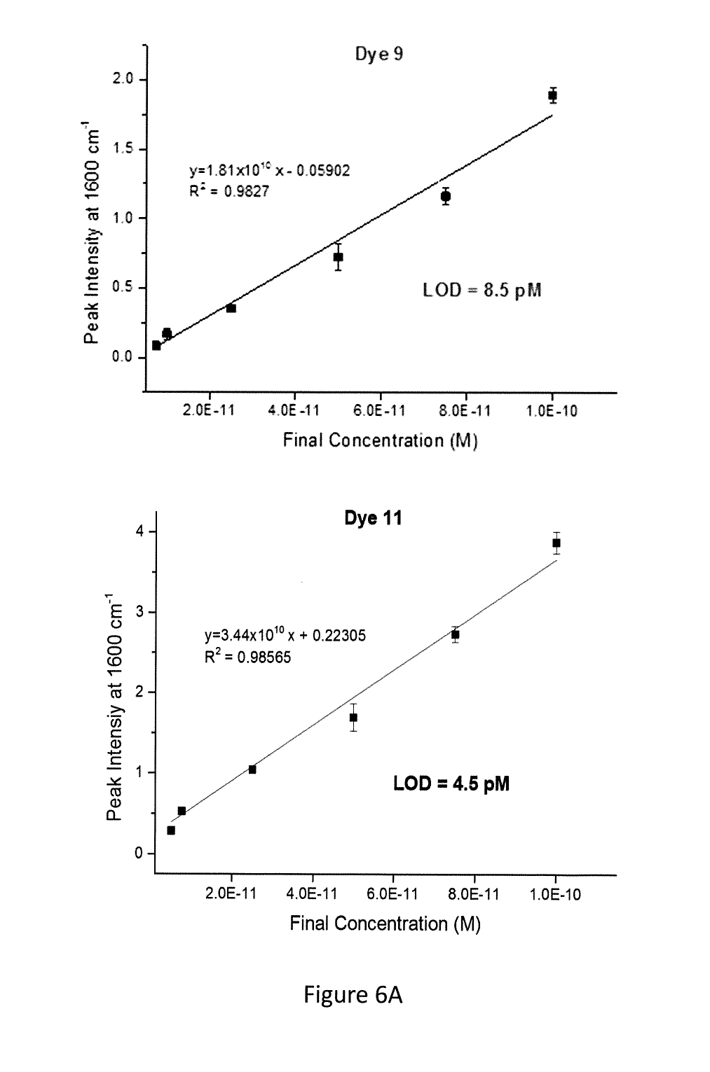

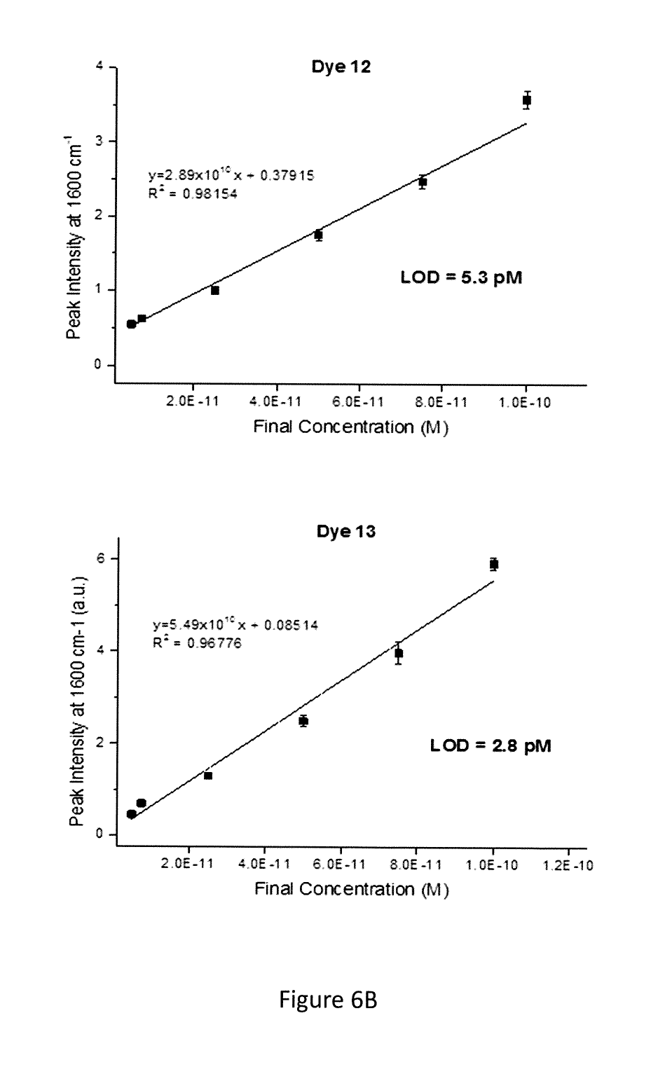

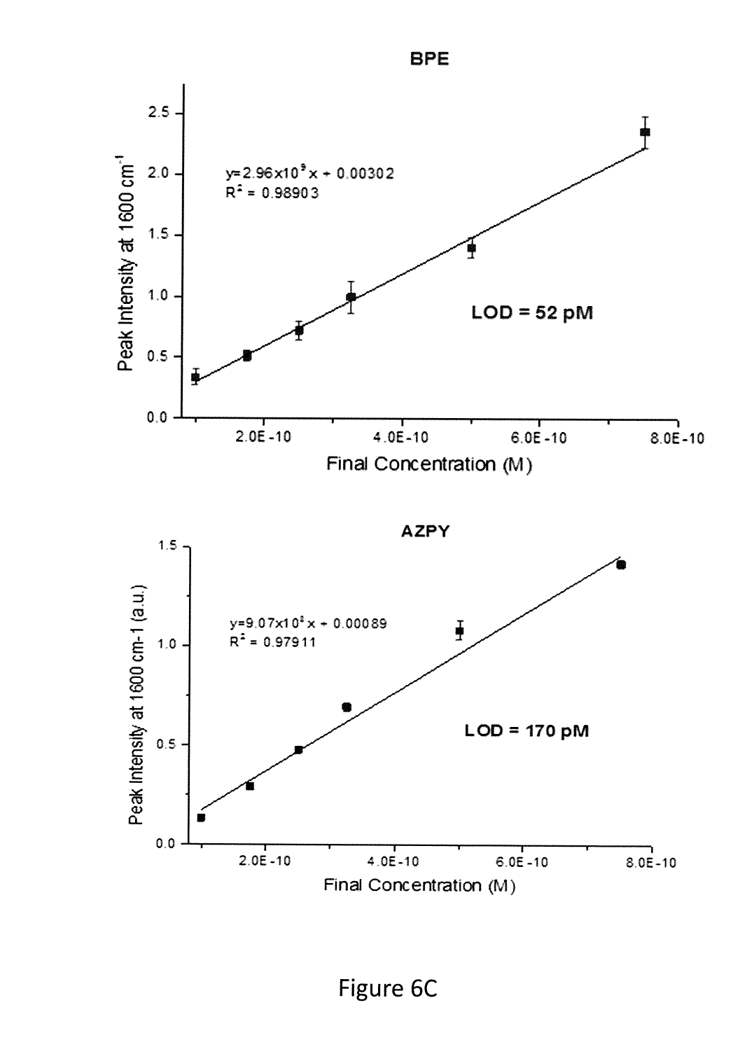

Not only do the dye-HGN assemblies give readable SERS spectra, the dye-HGN assemblies give low picomolar limits of detection. Limits of detection (LOD) for dye-HGN assemblies with dyes 9 and 11-13 are shown in FIG. 6 and are compared to the commercially available dyes BPE (bis(4-pyridyl)ethylene) and AZPY (4,4′-azopyridine), which have been used for 1064-nm excitation. LODs were calculated using the y=mx+c equation of the line; where y is 3 times the standard deviation of the blank. The dye-HGN assemblies with dyes 9 and 11-13 give much lower LODs (2.8-8.5 pM) than those with BPE (52 pM) and AZPY (170 pM).

Synthesis of HGNs for Use with 1280-nm Excitation. The HGN synthesis was carried out under inert conditions using a standard Schlenk line to prevent the cobalt nanoparticles from prematurely oxidizing. The method described was modified slightly from previous reports (The Journal of Physical Chemistry B, 2006, 110, 19935-19944; Nanoscale, 2013, 5, 765-771). In a typical synthesis, cobalt chloride hexahydrate (100 μL, 0.4M; Fisher Scientific, 99.99%) and trisodium citrate dihydrate (550 μL, 0.1 M; Sigma-Aldrich, >99%) were added into deionised water (100 mL) and degassed several times (10 mins vacuum and 15 mins argon). Sodium borohydride (1 mL, 0.1 M; Fisher Scientific, 99%) was injected into the solution and allowed to react for a further 20 minutes (under constant argon flow) until hydrogen evolution ceased, indicating complete hydrolysis of the reductant. The solution was degassed again (8 min vacuum and 10 min argon) before chloroauric acid trihydrate (33 mL, 248 μM; Fisher Scientific, ACS reagent grade) was injected. This mixture was allowed to react for an additional 10 minutes under argon with vigorous stirring. Before being exposed to air, were an obvious colour change from brown to green was observed. Finally, trisodium citrate (500 μL, 0.1 M) was added to stabilise the hollow gold nanoshell solution. Post synthesis, the HGN solution was concentrated through centrifugation (5000×g) and the precipitate was re-dispersed in trisodium citrate solution (2 mM) to give a final concentration of 2.97 nM. The HGNs had a localized surface plasmon resonance (SPR) at 720 nm.

Characterization and Use of HGNs with 1280-nm Excitation. Investigation into the SERS properties of the HGNs were carried out by mixing concentrated HGN solution (270 μL) with Raman reporter solution; namely dyes 1-14, BPE and AZPY (40 μL, 10 μM; synthesized in-house or purchased from Sigma-Aldrich) and potassium chloride (300 μL, 30 mM; Sigma-Aldrich). The Raman measurements were performed using a SnRI portable Raman spectrometer and a laser excitation wavelength of 1280 nm. All the measurements had a 7 second acquisition time and a laser power operating at 100 mW. Each sample was prepared in triplicate and 5 scans of each replicate were recorded. Furthermore, all the Raman spectra have been background corrected. For the SERS particle dilution study, the optimum conditions were used (as stated above) and deionised water was added to obtain subsequent concentrations, over the concentration range 1.3 nM to 80 pM. All other experimental conditions were kept the same as those stated above.

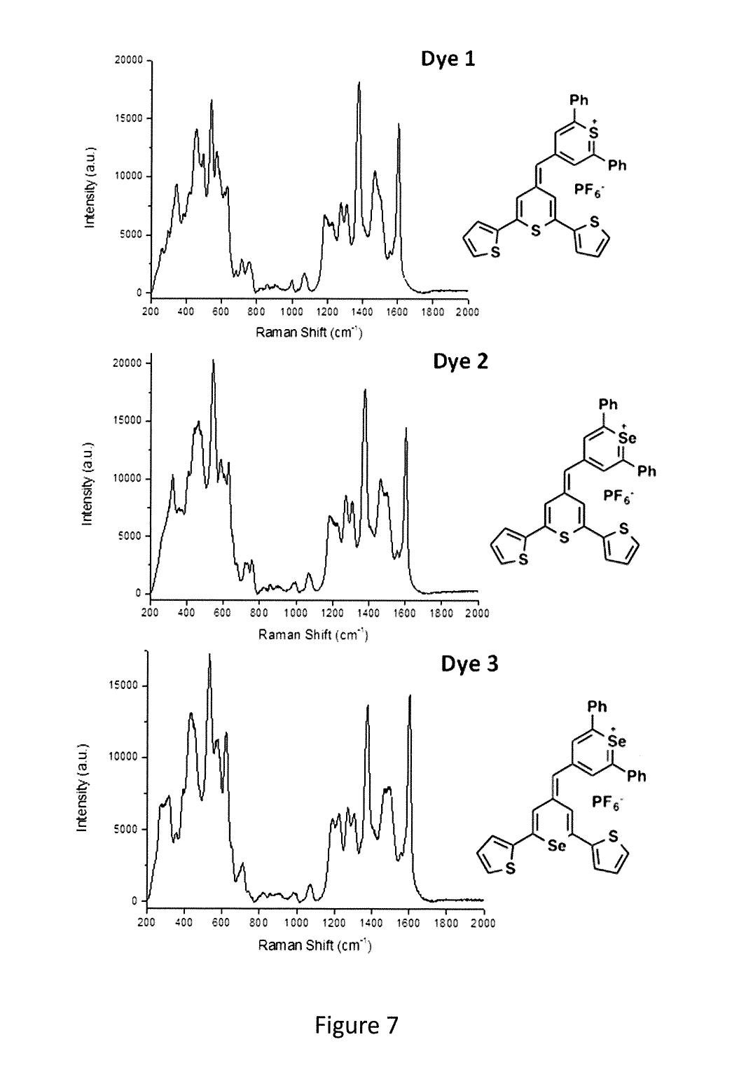

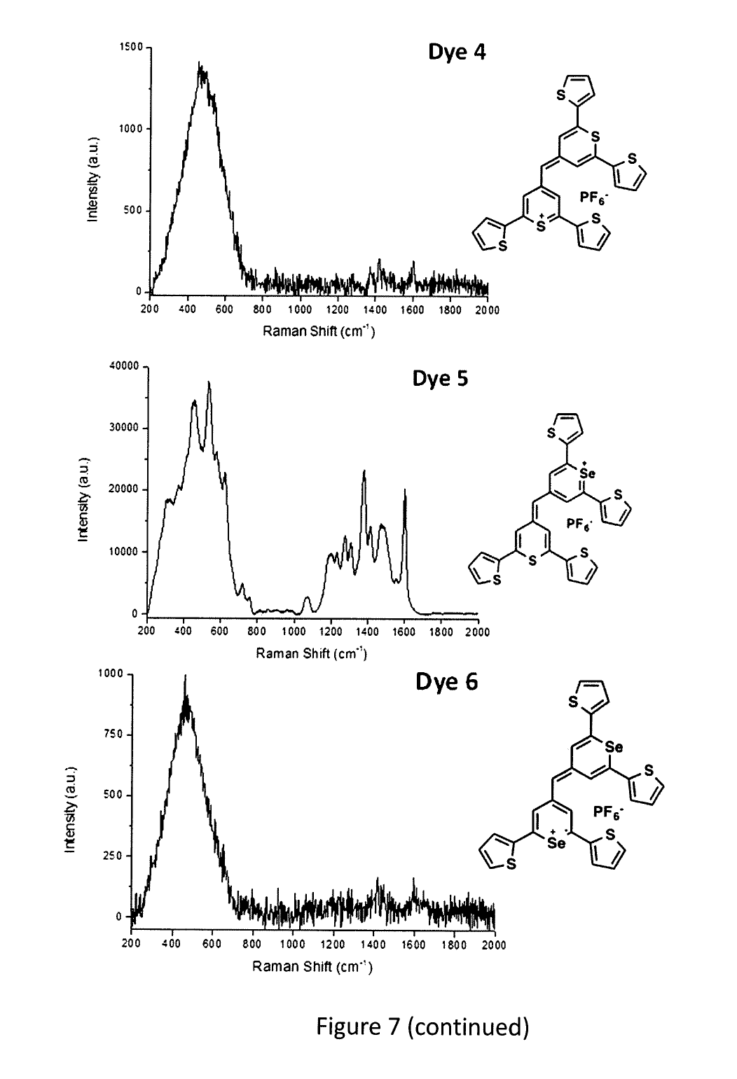

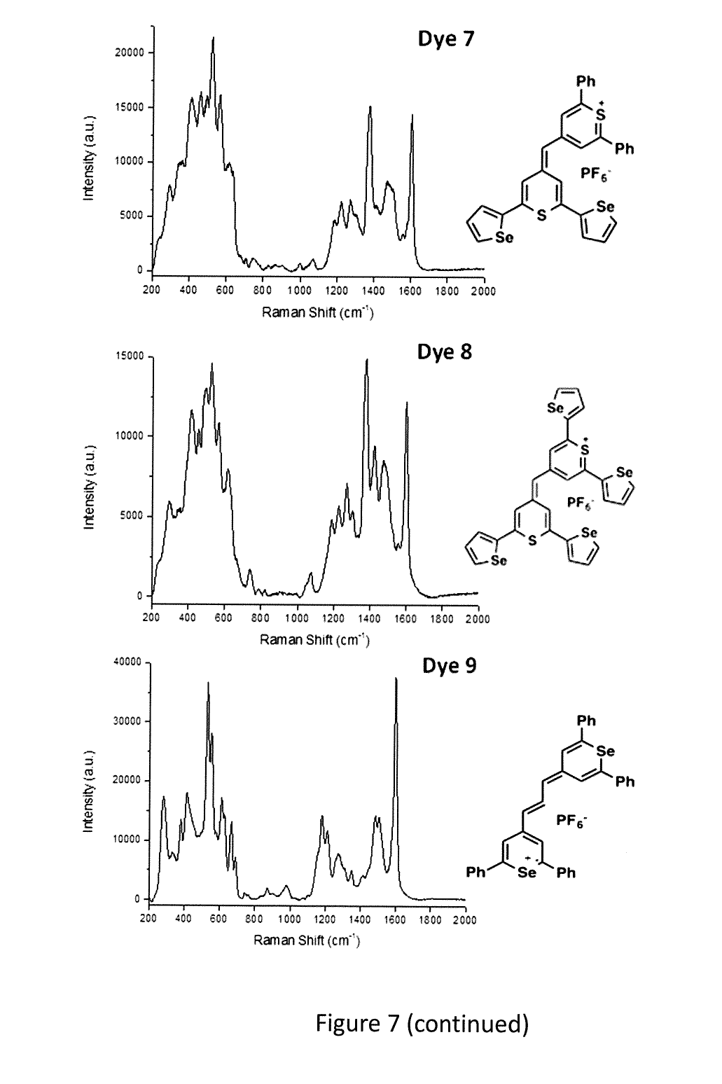

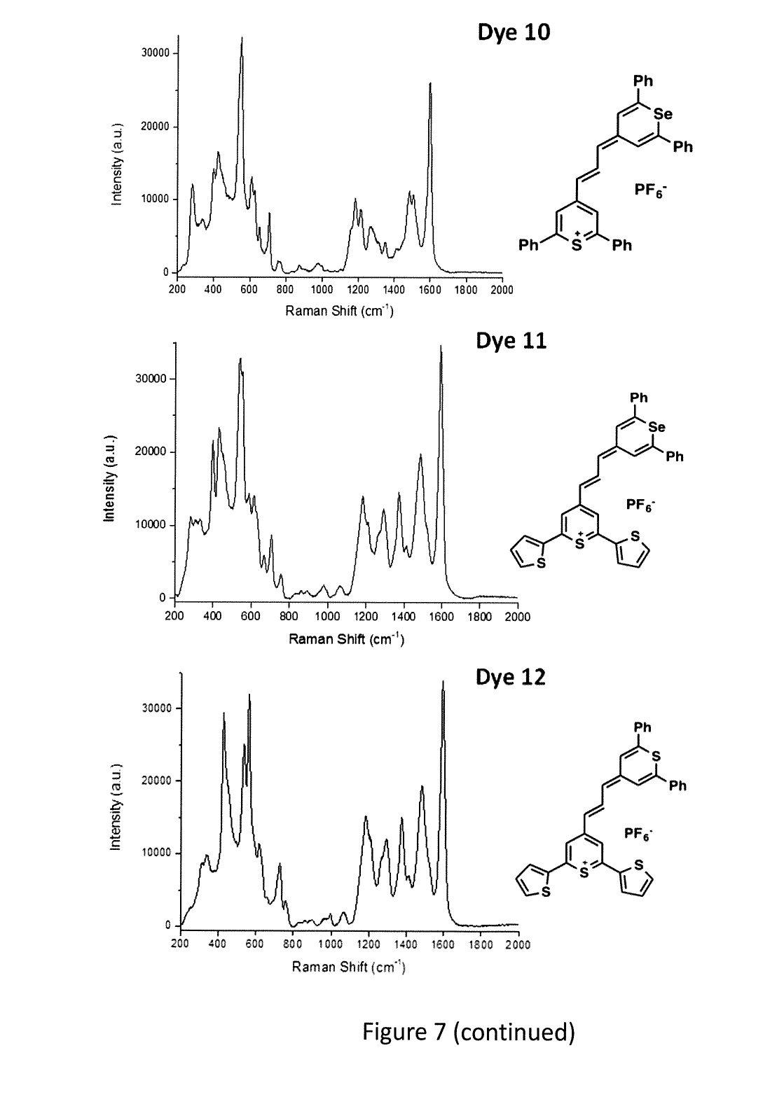

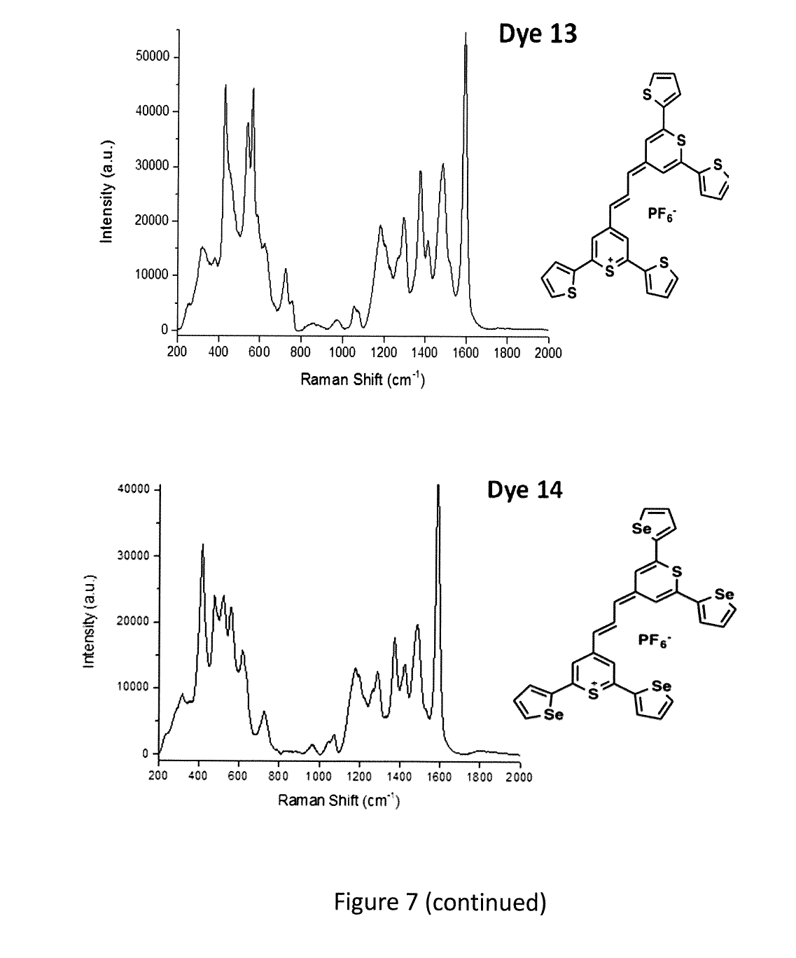

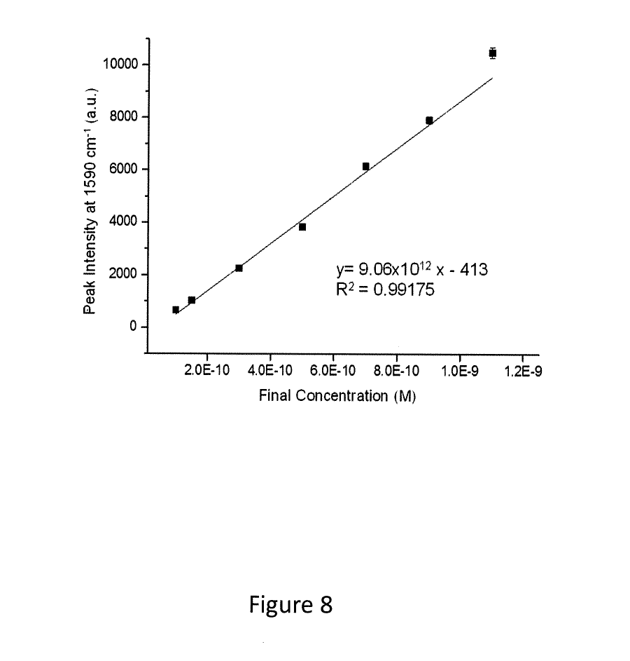

Examples of 1280-nm Excitation of Chalcogenopyrylium Dyes as SERS Reporters. The aggregated SERS spectra for the dye-HGN assemblies for dyes 1-14 with 1280-nm excitation is shown in FIG. 7. The dye 13-HGN assembly gave an 11.5 pM LOD as shown in FIG. 8. The LOD was calculated using the y=mx+c equation of the line; where y is 3 times the standard deviation of the blank.

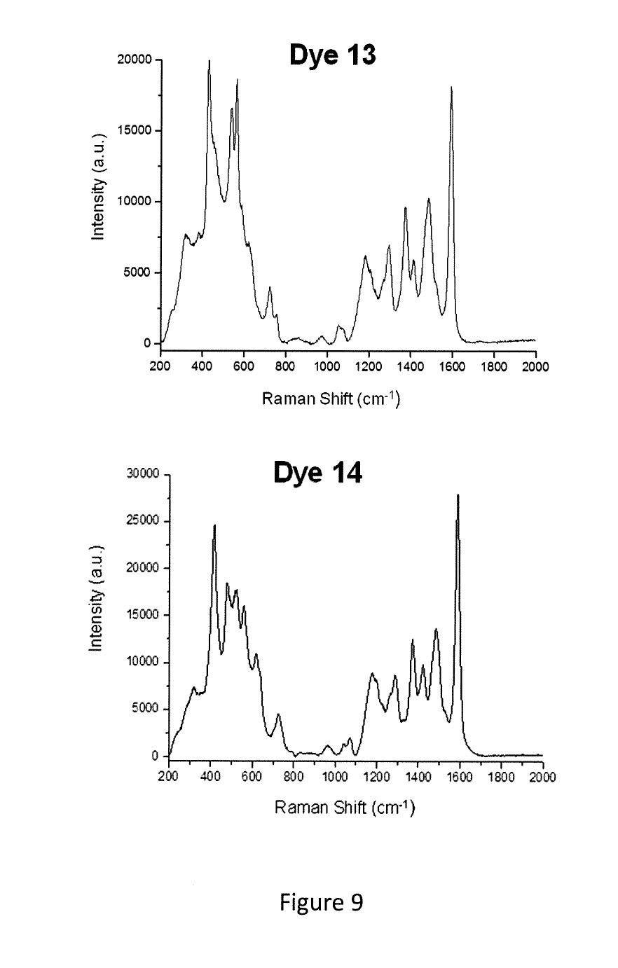

Dyes 13 and 14 with four 2-thienyl and four 2-selenophenyl substituents, respectively, did not require aggregation to give intense SERS signals. The unaggregated SERS spectra for the dye 13-HGN and dye 14-HGN assemblies with 1280-nm excitation is shown in FIG. 9.

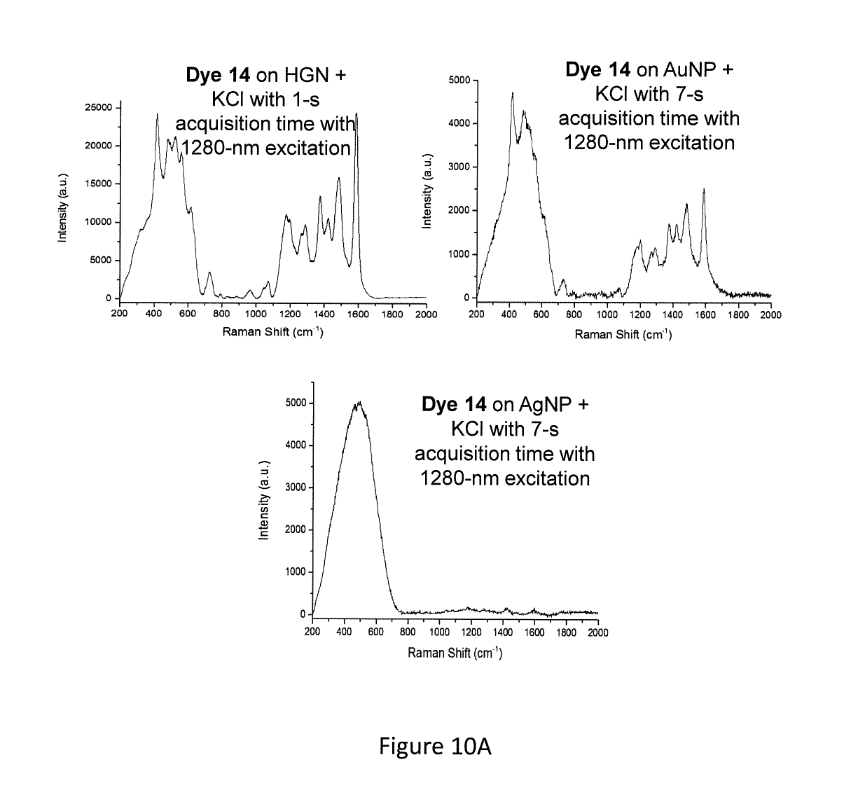

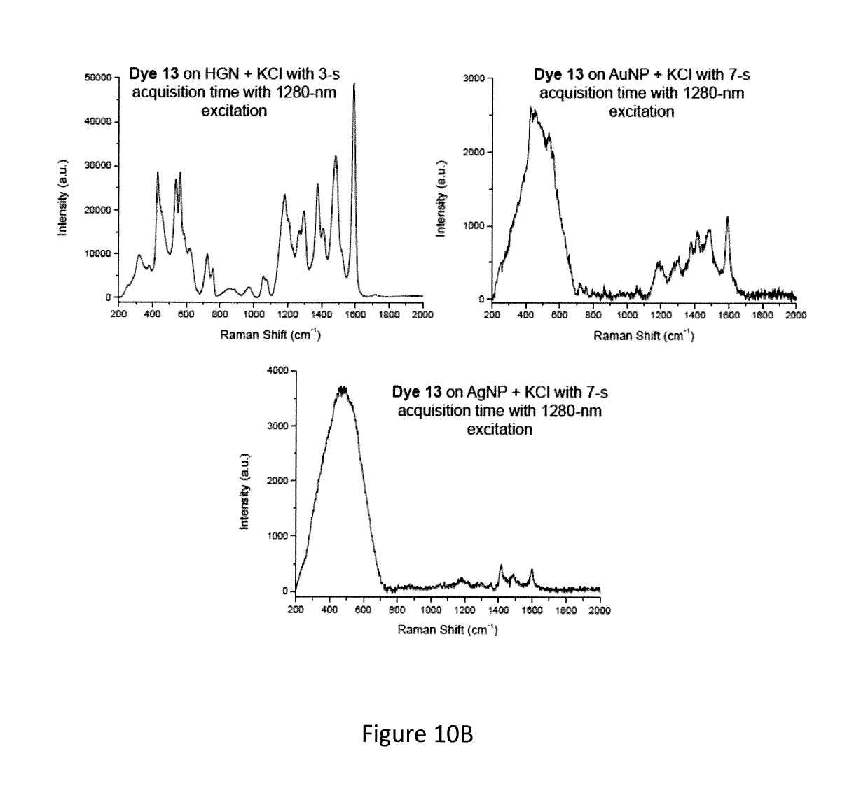

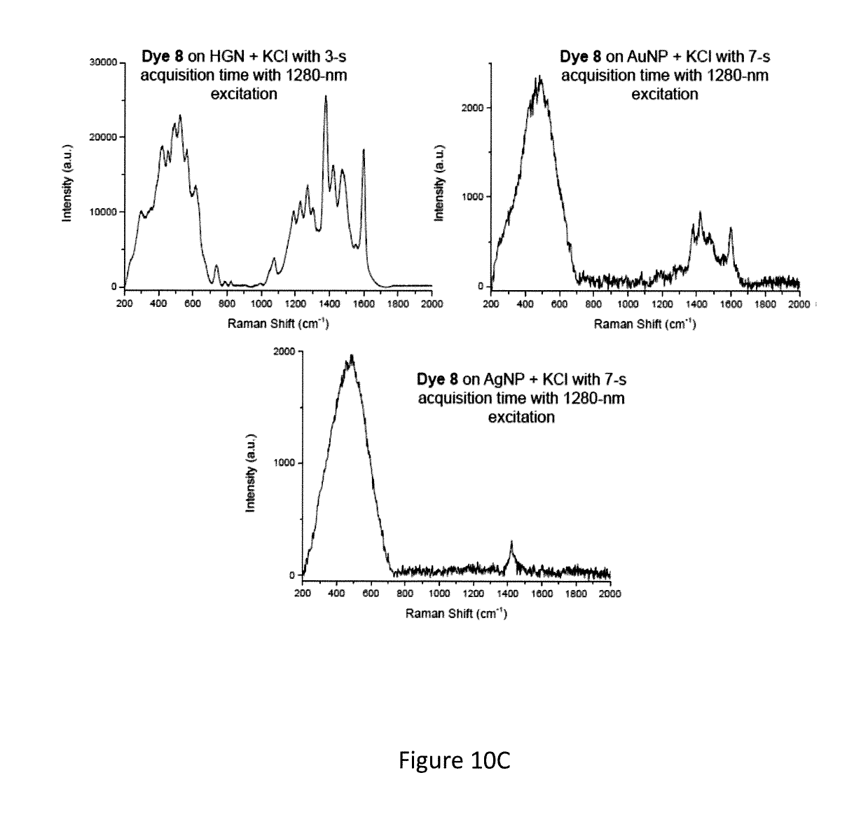

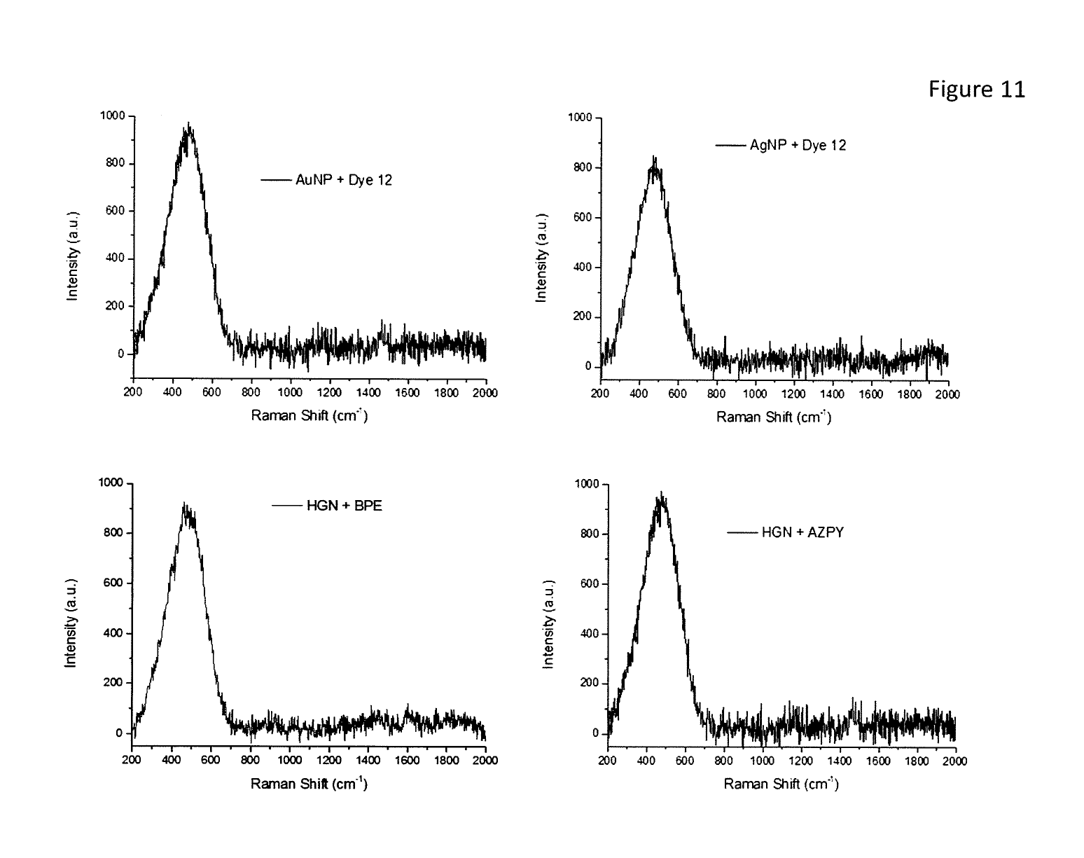

The dyes of this disclosure gave very weak SERS spectra with 1280-nm excitation on solid gold nanoparticles prepared as described above for FIG. 2 and similarly prepared solid silver nanoparticles. As shown in FIG. 10, these weak signals were obtained with dye 8, dye 13, and dye 14 of this disclosure and required long acquisition times (7 s). Other dyes of this disclosure as well as the commercially available dyes BPE (bis(4-pyridyl)ethylene) and AZPY (4,4′-azopyridine) have been successfully used as SERS reporters with 1064-nm excitation, but not with 1280-nm excitation even on HGNs. These results are summarized in FIG. 11 for dye 12, BPE, and AZPY and can be compared to the response of the dye 12-HGN assembly with 1280-nm excitation shown in FIG. 8. It was both surprising and unexpected that the dyes of this disclosure would give the strong signals observed in FIG. 8 as SERS reporters with HGNs with 1280-nm excitation.

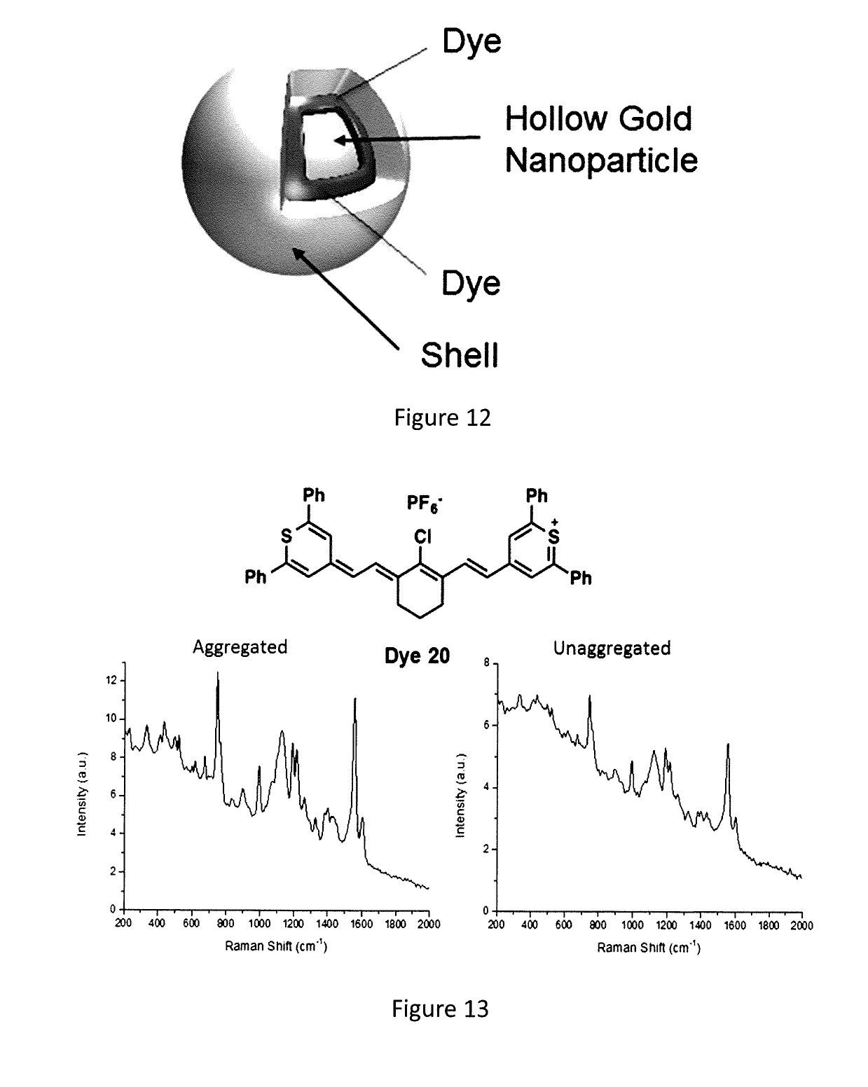

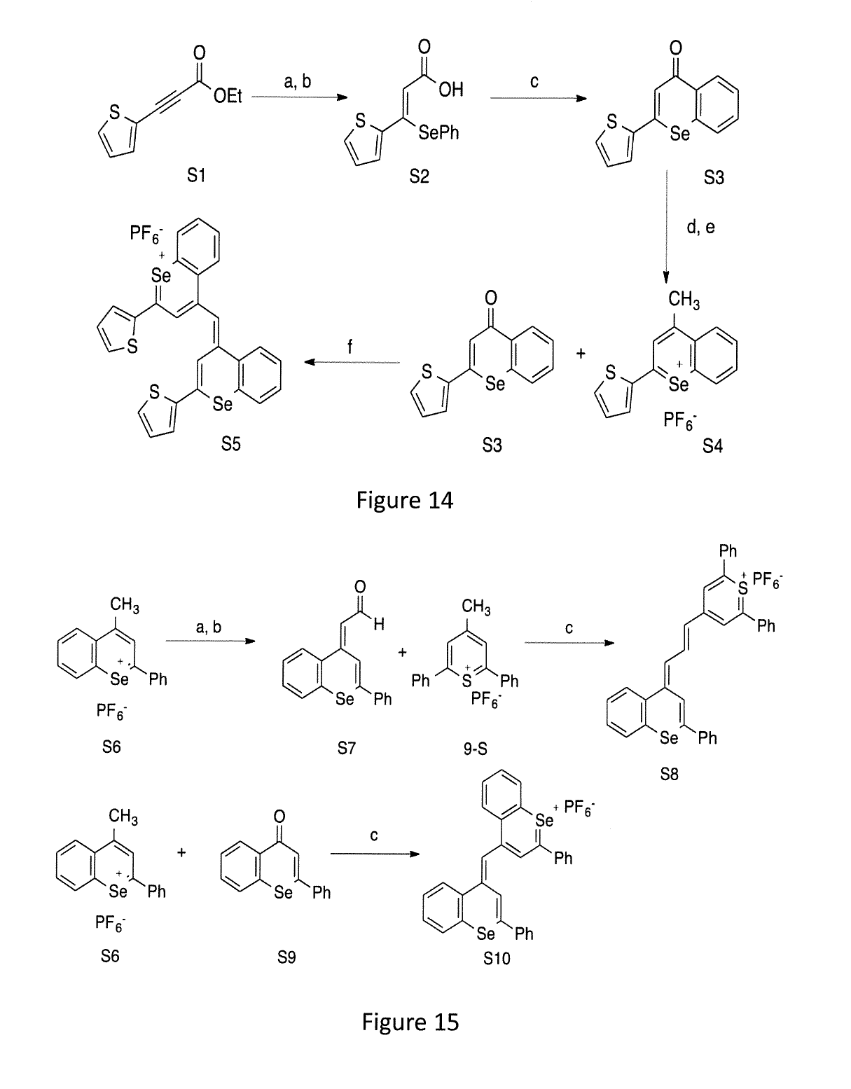

In imaging applications, the nanoparticle assemblies might be assembled as shown in FIG. 12. The dyes 1-14 are coated onto hollow gold nanoshells (HGNs). The dye-HGN assembly can be overcoated with polymeric materials such as a silica-based xerogel and targeting molecules for biological sites can be incorporated directly onto the HGN or in the polymeric overcoat.

Phenyl, 2-thienyl and 2-selenophenyl substituents can be incorporated into chalcogenopyrylium dyes absorbing at even longer wavelengths. If dyes absorb light at the wavelength of emission of the incident laser, the Raman reporters are in resonance with the incident laser and produce surface-enhanced resonance Raman scattering (SERRS), which can be orders of magnitude greater than the SERS response. To this end, we prepared dyes 18-21 (Chart 1, intermediates shown in Chart 2) as novel compositions of matter to show the feasibility of this approach. Dyes 18-21 show absorption maxima of 943 nm, 1001 nm, 1042, and 1119 nm, respectively (Table 1). Dye 20 is the hexafluorophosphate analogue of the commercially available tetrafluoroborate salt, which is sold as IR-1061.

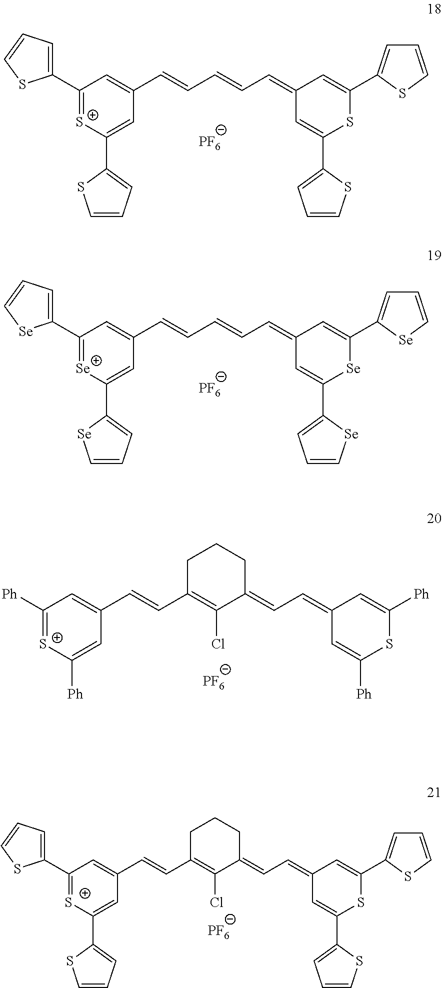

Chart 1 Longer-wavelength absorbing thiopyrylium and selenopyrylium dyes with four phenyl, 2-thienyl, or 2-selenophenyl substituents for use as SERS and SERRS reporters.

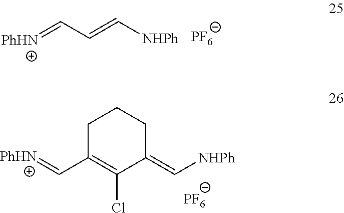

### Chart 2 Intermediates for Preparation of Dyes 18-20.

Aggregated and unaggregated dye 20-HGN assemblies were prepared as described for aggregated and unaggregated dye-HGN assemblies described with dyes 1-14. The aggregated and unaggregated SERS spectra for the dye 20-HGN assemblies with 1064-nm excitation are shown in FIG. 13.

Synthetic Methods. All reactions were performed open to air unless otherwise noted. Concentration in vacuo was performed on a rotary evaporator. NMR spectra were recorded at 300 or 500 MHz for ¹H and at 75.5 MHz for ¹³C with residual solvent signal as internal standard. UV/VIS-near-IR spectra were recorded in quartz cuvettes with a 1-cm path length. Melting points were determined with a capillary melting point apparatus and are uncorrected. Non-hygroscopic compounds have a purity of ≧95% as determined by elemental analyses for C, H, and N. Experimental values of C, H, and N are within 0.3% of theoretical values. ¹³C NMR was not recorded for pyrylium dyes due to limited solubility in common NMR solvents. Pyranones 16b-16d are known compounds (J. Heterocycl. Chem. 1999, 36, 707-717). The synthesis of thiopyranone 16a and 4-methylpyrylium salt 15c is shown in Scheme 2. 4-Methylpyrylium salts 15a, 15d, and 15e have been reported previously in the literature (J. Org. Chem. 1982, 47, 5235-5239; Organometallics 1988, 7, 1131-1147; Dyes Pigm. 2000, 45, 1-7).

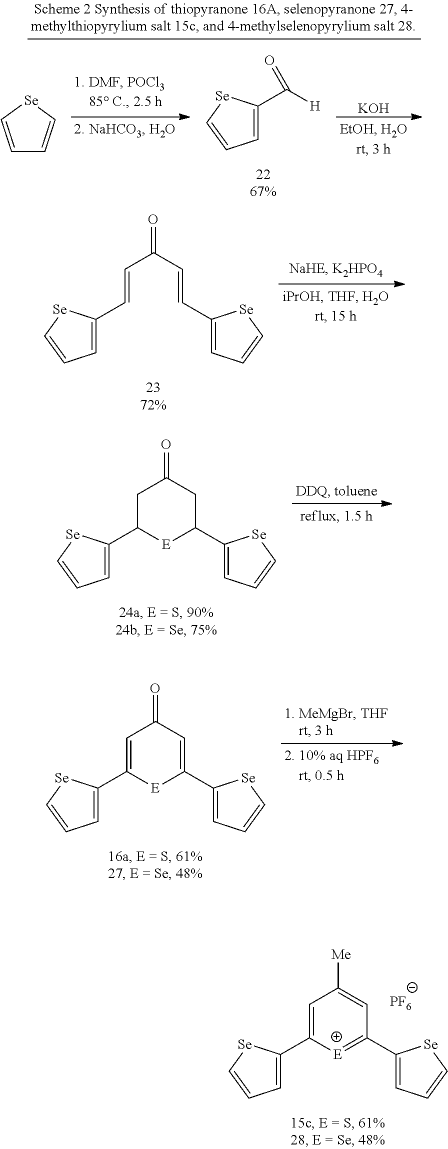

Synthesis of selenophen-2-carbaldehyde (22). Anhydrous DMF (15.0 mL) was added to a flame dried flask under argon and cooled to 0° C. POCl₃ (1.71 mL, 18.3 mmol) was added and the mixture allowed to stir for 0.5 h. The ice bath was removed, and selenophene (1.41 mL, 15.3 mmol) added. The reaction was heated to 85° C. and maintained at this temperature for 2.5 h. After cooling to ambient temperature, the mixture was poured into ice water (200 mL), neutralized with satd. NaHCO₃ (100 mL) and the product extracted with EtOAc (2×75 mL). The organic layer was dried with MgSO₄ and after concentration purified on SiO₂ with a 25% EtOAc/hexanes eluent to yield 1.64 g (67%) of a colorless oil: ¹H NMR [500 MHz, CDCl₃] δ 9.83 (s, 1H), 8.50 (d, 1H, J=5.5 Hz), 8.03 (d, 1H, J=4.0 Hz), 7.49-7.47 (m, 1H).

Synthesis of (1E,4E)-1,5-di(selenophen-2-yl)penta-1,4-dien-3-one (23). Selenophen-2-carbaldehyde (1.64 g, 10.3 mmol) and acetone (0.376 mL, 5.13 mmol) were dissolved in EtOH (10 mL). KOH (0.287 g, 5.13 mmol) dissolved in H₂O was added slowly to the stirring mixture and then allowed to stir for 3 hours at ambient temperature. The solution was diluted with H₂O (50 mL), and the product extracted with CH₂Cl₂ (2×50 mL). The organic layer was dried with MgSO₄, concentrated, and recrystallized from CH₂Cl₂/hexanes to yield 1.26 g (72%) of a yellow crystalline solid, mp 135-137° C.: ¹H NMR [500 MHz, CDCl₃] δ 8.09 (d, 2H, J=5.5 Hz), 7.85 (d, 2H, J=15.5 Hz), 7.52 (d, 2H, J=3.5 Hz), 7.30 (m, 2H), 6.70 (d, 2H, J=15.0 Hz); ¹³C NMR [75.5 MHz, CDCl₃] δ 187.53, 146.19, 137.84, 135.06, 133.90, 130.69, 125.67; HRMS (ESI) m/z 342.9131 (calcd for C₁₃H₁₀O⁸⁰Se₂+H⁺: 342.9135).

Synthesis of 2,6-di(selenophen-2-yl)tetrahydro-4H-thiopyran-4-one (24a). (1E,4E)-1,5-Di(selenophen-2-yl)penta-1,4-dien-3-one (1.20 g, 3.51 mmol) was dissolved in THF (5.0 mL). To this mixture isopropyl alcohol (10 mL), and K₂HPO₄ (0.961 g, 4.21 mmol) dissolved in H₂O were added followed by the addition of NaHS (0.356 g, 3.86 mmol). This was allowed to stir overnight at ambient temperature and under an argon atmosphere. The reaction was then diluted with H₂O (50 mL) and the product extracted with CH₂Cl₂ (2×50 mL). The organic layer was dried with MgSO₄, concentrated and then recrystallized from CH₂Cl₂/hexanes to yield 1.21 g (92%) of an off-white solid, mp 115-116° C.: ¹H NMR [300 MHz, CDCl₃] δ 7.97 (dd, 2H, J=5.3, 2.0 Hz), 7.21-7.17 (m, 4H), 4.71 (dd, 2H, J=12.3, 5.0 Hz), 3.14 (dd, 2H, J=13.8, 2.7 Hz), 2.95 (m, 2H); ¹³C NMR [75.5 MHz, CDCl₃] δ 205.63, 149.27, 130.91, 129.09, 127.09, 51.99, 45.45; HRMS (EI) m/z 375.8933 (calcd for C₁₃H₁₂OS⁸⁰Se₂: 375.8934).

Synthesis of 2,6-di(selenophen-2-yl)tetrahydro-4H-selenopyran-4-one (24b). Selenium powder (0.502 g, 6.36 mmol), NaBH₄ (0.481 g, 12.7 mmol), K₂HPO₄ (1.45 g, 6.36 mmol), H₂O (7.5 mL) and iPrOH (15 mL) were combined in a flask that had been flushed with argon and stirred for 15 min. (1E,4E)-1,5-di(selenophen-2-yl)penta-1,4-dien-3-one (1.45 g, 4.24 mmol) was dissolved in THF (7.5 mL) and added slowly to the stirring mixture. This was allowed to stir at ambient temperature for 1.5 h. The reaction was then diluted with H₂O (100 mL) and the product extracted with CH₂Cl₂ (3×50 mL). The organic layer was dried with MgSO₄, concentrated and then purified on SiO₂ with a CH₂Cl₂ eluent (R_{f}=0.60) to yield 1.33 g (74%) of a light yellow oil: ¹H NMR [500 MHz, CDCl₃] δ 7.98-7.96-7.95 (m, 2H), 7.17-7.11 (m, 4H), 4.96 (dd, 1H, 12.5, 3.0 Hz), 4.91 (t, 1H, J=6.5 Hz), 3.27-3.23 (m, 2H), 3.16 (t, 1H, J=13.0 Hz); ¹³C NMR [75.5 MHz, CDCl₃] δ 206.98, 206.69, 152.55, 150.55, 130.85, 130.68, 129.35, 129.24, 127.50, 126.83, 52.34, 50.78, 38.08, 36.22.

Synthesis of 2,6-di(selenophen-2-yl)-4H-thiopyran-4-one (16a). 2,6-Di(selenophen-2-yl)tetrahydro-4H-thiopyran-4-one (0.450 g, 1.20 mmol) was dissolved in anhydrous toluene (6.0 mL) and placed in a flame dried flask under argon. 2,3-Dichloro-5,6-dicyano-1,4-benzoquinone (0.679 g, 2.99 mmol) was added in one portion and the reaction refluxed for 1.5 h. This was cooled to ambient temperature, diluted with CH₂Cl₂ (50 mL) and the mixture washed with satd. aqueous NaHCO₃ (50 mL). The organic layer was separated, dried with MgSO₄, and after concentration purified on SiO₂ with a 20% EtOAc/CH₂Cl₂ eluent (R_{f}=0.47) to yield 0.273 g (61%) of a light brown solid, mp 138-140° C.: ¹H NMR [500 MHz, CDCl₃] δ 8.19 (dd, 2H, J=5.5, 1.5 Hz), 7.69 (dd, 2H, J=4.0, 1.5 Hz), 7.38 (m, 2H), 7.09 (s, 2H); ¹³C NMR [75.5 MHz, CDCl₃] δ 182.13, 146.78, 143.61, 134.77, 130.82, 129.51, 125.67; HRMS (ESI) m/z 375.8699 (calcd for C₁₃H₈OS⁸⁰Se₂+H⁺: 372.8698).

Synthesis of 2,6-di(selenophen-2-yl)-4H-thiopyran-4-one (27). 2,6-di(selenophen-2-yl)tetrahydro-4H-selenopyran-4-one (0.386 g, 0.911 mmol) was dissolved in anhydrous toluene (7.5 mL) and placed in a flame dried flask under argon. 2,3-Dichloro-5,6-dicyano-1,4-benzoquinone (0.517 g, 2.28 mmol) was added in one portion and the reaction refluxed for 2 h. The reaction was cooled to ambient temperature, diluted with CH₂Cl₂ (50 mL) and the mixture washed with satd. aqueous NaHCO₃ (50 mL). The organic layer was separated and the product extracted with additional CH₂Cl₂ (2×50 mL). The organic layer was dried with MgSO₄, and after concentration purified on SiO₂ with a 20% EtOAc/CH₂Cl₂ eluent (R_{f}=0.56) to yield 0.184 g (48%) of a light brown solid: ¹H NMR [500 MHz, CDCl₃] δ 8.19 (d, 2H, J=5.5 Hz), 7.62 (d, 2H, J=3.5 Hz), 7.38 (t, 2H, J=5.0 Hz), 7.16 (s, 2H); ¹³C NMR [75.5 MHz, CDCl₃] δ 184.16, 147.44, 145.35, 134.65, 130.75, 129.55, 126.94.

Synthesis of 4-methyl-2,6-di(thiophen-2-yl)thiopyrylium hexafluorophosphate (15a). 2,6-Bis(thiophen-2-yl)-4H-thiopyran-4-one (0.288 g, 1.04 mmol) was dissolved in anhydrous THF (7.0 mL) in a flame dried flask under argon. 3.0 M MeMgBr (1.04 mL, 3.12 mmol) was added dropwise to this solution and allowed to stir at ambient temperature for 2 h. The solution was poured into 10% aqueous HPF₆ (50 mL) and allowed to stir for 15 min before the solid was isolated by filtration. The resulting solid was dissolved in CH₂Cl₂, dried with Na₂SO₄, and the solvent removed under reduced pressure. The product was then recrystallized from CH₃CN/ether to yield 0.381 g (87%) of a bright red solid, mp 190-192° C.: ¹H NMR [500 MHz, CD₃CN] δ 8.35 (s, 2H), 8.08-8.07 (m, 4H), 7.39 (t, 2H, J=5.0 Hz), 2.78 (s, 3H); ¹³C NMR [75.5 MHz, CD₃CN] δ 167.63, 159.95, 137.91, 137.18, 133.96, 131.84, 131.21, 25.92; HRMS (ESI) m/z 275.0021 (calcd for C₁₄H₁₁S₃⁺: 275.0017).

Synthesis of 4-methyl-2,6-bis(thiophen-2-yl)selenopyrylium hexafluorophosphate (15b). In a flame-dried flask under argon, 2,6-bis(thiophen-2-yl)-4H-selenopyran-4-one (0.200 g, 0.621 mmol), was dissolved in anhydrous THF (5.0 mL). 3.0 M MeMgBr (0.620 mL, 1.86 mmol) was added dropwise and the solution allowed to stir at ambient temperature for 0.5 h. The mixture was quenched with MeOH (1 mL), poured into 10% aqueous HPF₆ (50 mL), and extracted with CH₂Cl₂ (3×25 mL). The organic layer was dried with Na₂SO₄, concentrated under reduced pressure, and then recrystallized from CH₃CN/ether to yield 0.239 g (82%) of a red solid, mp 185-187° C.: ¹H NMR [500 MHz, CD₃CN] δ 8.23 (s, 4H), 8.11 (d, 2H, J=5.5 Hz), 8.02 (d, 2H, J=4.5 Hz), 7.39 (t, 2H, J=4.0 Hz), 2.70 (s, 3H); ¹³C NMR [75.5 MHz, CD₃CN] δ 168.23, 167.68, 139.67, 134.04, 132.10, 131.35, 27.31; HRMS (ESI) m/z 322.9469 (calcd for C₁₄H₁₁S₂⁸⁰Se⁺: 322.9462).

Synthesis of 4-methyl-2,6-di(selenophen-2-yl)thiopyrylium hexafluorophosphate (15c). 2,6-Di(selenophen-2-yl)-4H-thiopyran-4-one (0.300 g, 0.807 mmol) was dissolved in anhydrous THF (8.0 mL) in a flame dried flask under argon. 3.0 M MeMgBr (0.807 mL, 2.42 mmol) was added dropwise and the reaction stirred at ambient temperature for 1 h. The reaction was poured into 10% aqueous HPF₆ (40 mL), and stirred for 10 min. The resulting solid was extracted with a mixture of CH₂Cl₂ (50 mL) and CH₃CN (5.0 mL), dried with Na₂SO₄, and after concentration recrystallized from CH₃CN/ether to yield 0.312 g (75%) of a bright red solid, mp >260° C.: ¹H NMR [500 MHz, CD₃CN] δ 8.78 (d, 2H, J=5.5 Hz), 8.26-8.25 (m, 4H), 7.61 (t, 2H, J=4.5 Hz), 2.76 (s, 3H); ¹³C NMR [75.5 MHz, CDCl₃] δ 167.42, 162.17, 144.98, 142.11, 136.62, 133.88, 132.19, 25.77; HRMS (EI) m/z 370.8900 (calcd for C₁₄H₁₁S⁸⁰Se₂⁺: 370.8906).

Synthesis of 4-methyl-2,6-di(selenophen-2-yl)selenopyrylium hexafluorophosphate (28). 2,6-di(selenophen-2-yl)-4H-selenopyran-4-one (0.750 g, 1.79 mmol) was dissolved in anhydrous THF (9.0 mL) in a flame dried flask under argon. 3.0 M MeMgBr (1.79 mL, 3.02 mmol) was added dropwise and the reaction stirred at ambient temperature for 1 h. The reaction was poured into 10% aqueous HPF₆ (50 mL), and stirred for 30 min. The resulting solid was extracted with CH₂Cl₂ (50 mL) with the aid of CH₃CN (10 mL), dried with Na₂SO₄, and after concentration recrystallized from CH₃CN/ether to yield 0.789 g (78%) of a dark red solid: ¹H NMR [500 MHz, CD₃CN] δ 8.82 (d, 2H, J=6.0 Hz), 8.19 (d, 2H, J=4.0 Hz), 8.12 (s, 2H), 7.62-7.60 (m, 2H), 2.68 (s, 3H).

Synthesis of 4-(2,6-diphenyl-4H-thiopyran-4ylidene)acetaldehyde (17a). 4-Methyl-2,6-di(phenyl)selenopyrylium hexafluorophosphate (0.200 g, 0.439 mmol), N,N-dimethylthioformamide (0.112 mL, 1.32 mmol) and Ac₂O (4.0 mL) were added to a round-bottom flask and heated at 95° C. for 90 min. After cooling to ambient temperature CH₃CN (4.0 mL) was added and the product precipitated by addition of ether and chilling overnight in the freezer. The iminium salt was isolated by filtration, and hydrolyzed by dissolving in CH₃CN (4.0 mL), adding satd. aqueous NaHCO₃ (4.0 mL) and heating the mixture to 80° C. over a 15 min period. The reaction was maintained at this temperature for 0.5 h, after which the reaction was diluted with H₂O (50 mL), the product extracted with CH₂Cl₂ (3×30 mL), dried with MgSO₄, and after concentration purified on SiO₂ with first a CH₂Cl₂ and then a 10% EtOAc/CH₂Cl₂ (R_{f}=0.70) eluent to yield 0.122 g (82%) of a orange oil: ¹H NMR [500 MHz, CDCl₃] δ 10.11 (d, 1H, J=10.5 Hz), 8.32 (s, 1H), 7.62-7.46 (m, 9H), 7.00 (s, 1H), 5.88 (d, 1H, J=11.0 Hz); ¹³C NMR [75.5 MHz, CDCl₃] δ 188.69, 148.44, 147.01, 146.03, 138.65, 138.40, 129.96, 129.90, 129.14, 126.62, 126.44, 126.37, 126.31, 125.67, 120.57, 120.48; HRMS (EI) m/z 339.0292 (calcd for C₁₉H₁₅O⁸⁰Se: 339.0283).

Synthesis of 4-(2,6-di(thiophene-2-yl)-4H-thiopyran-4ylidene)acetaldehyde (17b). 4-Methyl-2,6-di(thiophen-2-yl)thiopyrylium hexafluorophosphate (0.350 g, 0.833 mmol), N,N-dimethylthioformamide (0.213 mL, 2.50 mmol) and Ac₂O (3.0 mL) were combined in a small round bottom flask and heated at 95° C. for 1 h. After cooling to ambient temperature an additional portion of Ac₂O (2.0 mL) was added and the solution diluted with ether. The formed iminium salt was allowed to precipitate in the freezer overnight, and then isolated by filtration to yield a bright orange solid. This solid was dissolved in CH₃CN (3.0 mL) and satd. aqueous NaHCO₃ (3.0 mL) was added. This mixture was heated to 80° C. over 15 min, and kept at that temperature for 0.5 h. After diluting with H₂O (30 mL) the product was extracted with CH₂Cl₂ (3×50 mL), dried with Na₂SO₄ and purified on SiO₂ with a 10% EtOAc/CH₂Cl₂ eluent (R_{f}=0.71) to yield a yellow oil that was recrystallized in CH₂Cl₂/hexanes to yield 0.219 g (87%) of a yellow crystalline solid, mp 143-144° C.: ¹H NMR [500 MHz, CDCl₃] δ 9.84 (d, 1H, J=6.0 Hz), 8.26 (s, 1H), 7.45-7.39 (m, 4H), 7.13-7.11 (m, 2H), 6.88 (s, 1H), 5.72 (d, 1H, J=6.5 Hz); ¹³C NMR [75.5 MHz, CDCl₃] δ 188.05, 146.43, 139.36, 139.07, 137.33, 136.65, 128.16, 127.78, 127.58, 126.30, 126.01, 122.48, 117.63, 117.48; HRMS (EI) m/z 302.9971 (calcd for C₁₅H₁₁O₁S₃: 302.9967).

Synthesis of 2-(2,6-di(selenophen-2-yl)-4H-thiopyran-4-ylidene)acetaldehyde (17c). 4-Methyl-2,6-di(selenophen-2-yl)thiopyrylium hexafluorophosphate (0.150 g, 0.291 mmol), N,N-dimethylthioformamide (74.3 μL, 0.872 mmol) and Ac₂O (2.0 mL) were added to a round-bottom flask and heated at 95° C. for 90 min. After cooling to ambient temperature, Ac₂O (2.0 mL) was added and the product precipitated by addition of ether and chilling overnight in the freezer. The iminium salt was isolated by filtration, and hydrolyzed by dissolving in CH₃CN (3.0 mL), adding satd. aqueous NaHCO₃ (3.0 mL) and heating the mixture to 80° C. over a 15 min period. The reaction was maintained at this temperature for ½ h, after which the reaction was diluted with H₂O (30 mL), the product extracted with CH₂Cl₂ (3×20 mL), dried with MgSO₄, and after concentration purified on SiO₂ with a 10% EtOAc/CH₂Cl₂ (R_{f}=0.62) eluent to yield 80.3 mg (69%) of a brown solid, mp 145-146° C.: ¹H NMR [500 MHz, CDCl₃] δ 9.95 (d, 1H, J=6.0 Hz), 8.21 (s, 1H), 8.13-8.10 (m, 2H), 7.62 (d, 1H, J=4.0 Hz), 7.57 (d, 1H, J=4.0 Hz), 7.36-7.33 (m, 2H), 6.81 (s, 1H), 5.72 (d, 1H, J=6.0 Hz); ¹³C NMR [75.5 MHz, CDCl₃] δ 188.10, 146.72, 144.92, 144.58, 139.48, 138.77, 133.47, 133.21, 130.57, 128.62, 128.34, 123.32, 118.53, 117.38; HRMS (ESI) m/z 398.8861 (calcd for C₁₅H₁₀OS⁸⁰Se₂+H⁺: 398.8856).

Synthesis of 4-(2,6-diphenyl-4H-thiopyran-4-ylidene)methyl)-2,6-di(thiophen-2-yl)thiopyrylium hexafluorophosphate (Dye 1). 4-Methyl-2,6-di(thiophen-2-yl)thiopyrylium hexafluorophosphate (50.0 mg, 0.119 mmol), 2,6-bis(phenyl)-4H-thiopyran-4-one (34.6 mg, 0.131 mmol) and Ac₂O (2.0 mL) were heated for 5 min at 105° C. prior to cooling to ambient temperature, diluting with CH₃CN (3.0 mL) and adding ether to precipitate the product. Yielded 77.6 mg (91%) of a copper bronze solid, mp 222-223° C.: ¹H NMR [500 MHz, CD₂Cl₂] δ 8.04 (br s, 2H), 7.90 (br s, 2H), 7.83 (d, 4H, J=8.0 Hz), 7.75-7.73 (m, 4H), 7.70-7.63 (m, 6H), 7.30 (t, 2H, J=4.5 Hz), 6.67 (s, 1H); Anal. Calcd for C₃₁H₂₁S₄.PF₆: C, 55.85; H, 3.17. Found: C, 55.90; H, 3.29; HRMS (ESI) m/z 521.0518 (calcd for C₃₁H₂₁S₄⁺: 521.0521); λₘₐₓ (CH₂Cl₂)=653 nm, ε=1.3×10⁵ M⁻¹ cm⁻¹; 473 nm ε=1.6×10⁴ M⁻¹ cm¹; 410 nm ε=1.4×10⁴ M⁻¹ cm¹.

Synthesis of 4-((2,6-diphenyl-4H-selenopyran-4-ylidene)methyl)-2,6-di(thiophen-2-yl)thiopyrylium hexafluorophosphate (Dye 2). 4-Methyl-2,6-di(thiophen-2-yl)thiopyrylium hexafluorophosphate (50.0 mg, 0.119 mmol), 2,6-bis(phenyl)-4H-selenopyran-4-one (40.6 mg, 0.131 mmol) and Ac₂O (2.0 mL) were heated for 5 min at 105° C. prior to cooling to ambient temperature, diluting with CH₃CN (3.0 mL) and adding ether to precipitate the product. Yielded 77.6 mg (91%) of a copper bronze solid, mp 249-250° C.: ¹H NMR [500 MHz, CD₂Cl₂] δ 8.02 (br s, 2H), 7.94 (s, 2H), 7.78-7.75 (m, 8H), 7.67-7.60 (m, 6H), 7.30 (t, 2H, J=5.0 Hz), 6.76 (s, 1H); Anal. Calcd for C₃₁H₂₁S₃Se.PF₆½H₂O:C, 51.53; H, 3.07. Found: C, 51.55; H, 3.01; HRMS (ESI) m/z 568.9958 (calcd for C₃₁H₂₁S₃⁸⁰Se⁺: 568.9965); λₘₐₓ (CH₂Cl₂)=676 nm, ε=1.3×10⁵ M⁻¹ cm⁻¹; 483 nm, ε=1.6×10⁴ M⁻¹ cm⁻¹; 422 nm, ε=1.5×10⁴ M⁻¹ cm⁻¹.

Synthesis of 4-((2,6-diphenyl-4H-selenopyran-4-ylidene)methyl)-2,6-di(thiophen-2-yl)selenopyrylium hexafluorophosphate (Dye 3). 4-Methyl-2,6-di(thiophen-2-yl)selenopyrylium hexafluorophosphate (50.0 mg, 0.107 mmol), 2,6-bis(phenyl)-4H-selenopyran-4-one (36.6 mg, 0.118 mmol) and Ac₂O (4.0 mL) were heated for 90 sec at 105° C. prior to cooling to ambient temperature, diluting with CH₃CN (3.0 mL) and adding ether to precipitate the product. Product was purified on SiO₂ with a 10% EtOAc/CH₂Cl₂ eluent (R_{f}=0.34) to yield 35.7 mg (44%) of a copper bronze solid, mp 243-244° C.: ¹H NMR [500 MHz, CD₂Cl₂] δ 8.06 (br s, 2H), 7.93 (s, 2H), 7.79 (d, 4H, J=7.5 Hz), 7.75 (d, 2H, J=5.5 Hz), 7.68-7.65 (m, 4H), 7.62 (t, 4H, J=7.0 Hz), 7.29 (t, 2H, J=4.5 Hz), 6.86 (s, 1H); Anal. Calcd for C₃₁H₂₁S₂Se₂.PF₆: C, 48.96; H, 2.78. Found: C, 48.68; H, 2.76; HRMS (ESI) m/z 616.9402 (calcd for C₃₁H₂₁S₂⁸⁰Se₂⁺: 616.9410); λₘₐₓ (CH₂Cl₂)=699 nm, ε=1.5×10⁵ M⁻¹ cm⁻¹; 499 nm, ε=1.9×10⁴ M⁻¹ cm⁻¹; 428 nm ε=1.8×10⁴ M⁻¹ cm⁻¹.

Synthesis of 4-((2,6-di(thiophen-2-yl)-4H-thiopyran-4-ylidene)methyl)-2,6-di(thiophen-2-yl)thiopyrylium hexafluorophosphate (Dye 4). 4-Methyl-2,6-di(thiophen-2-yl)thiopyrylium hexafluorophosphate (50.0 mg, 0.119 mmol), 2,6-bis(thiophen-2-yl)-4H-thiopyran-4-one (36.2 mg, 0.131 mmol) and Ac₂O (2.0 mL) were heated for 20 min at 105° C. prior to cooling to rt, diluting with CH₃CN (3.0 mL) and adding ether to precipitate the product. Yielded 78.6 mg (97%) of a copper bronze solid, mp >260° C.: ¹H NMR [500 MHz, CD₂Cl₂] δ 7.86 (br s, 4H), 7.75 (d, 8H, J=4.5 Hz), 7.30 (t, 4H, J=4.5 Hz), 6.61 (s, 1H); Anal. Calcd for C₂₇H₁₇S₆.PF₆: C, 47.78; H, 2.52. Found: C, 47.94; H, 2.44; HRMS (ESI) m/z 532.9628 (calcd for C₂₇H₁₇S₆⁺: 532.9649); λₘₐₓ (CH₂Cl₂)=676 nm, ε=1.2×10⁵ M⁻¹ cm⁻¹; 480 nm, ε=2.7×10⁴ M⁻¹ cm⁻¹.

Synthesis of 4-((2,6-di(thiophen-2-yl)-4H-thiopyran-4-ylidene)methyl)-2,6-di(thiophen-2-yl)selenopyrylium hexafluorophosphate (Dye 5). 4-Methyl-2,6-di(thiophen-2-yl)thiopyrylium hexafluorophosphate (50.0 mg, 0.119 mmol), 2,6-bis(thiophen-2-yl)-4H-selenopyran-4-one (42.3 mg, 0.131 mmol) and Ac₂O (2.0 mL) were heated for 5 min at 105° C. prior to cooling to ambient temperature, diluting with CH₃CN (3.0 mL) and adding ether to precipitate the product. Yielded 83.2 mg (96%) of a copper bronze solid, mp 254-256° C.: ¹H NMR [500 MHz, CD₂Cl₂] δ 7.94 (s, 2H), 7.89 (br s, 2H), 7.83-7.87 (m, 6H), 7.71 (dd, 2H, J=4.0, 1.0 Hz), 6.73 (s, 1H); Anal. Calcd for C₂₇H₁₇S₅Se.PF₆: C, 44.69; H, 2.36. Found: C, 44.76; H, 2.49; HRMS (ESI) m/z 580.9087 (calcd for C₂₇H₁₇S₅⁸⁰Se⁺: 580.9094); λₘₐₓ (CH₂Cl₂)=698 nm, ε=1.1×10⁵ M⁻¹ cm⁻¹; 493 nm, ε=2.5×10⁴ M⁻¹ cm⁻¹.

Synthesis of 4-((2,6-di(thiophen-2-yl)-4H-selenopyran-4-ylidene)methyl)-2,6-di(thiophen-2-yl)selenopyrylium hexafluorophosphate (Dye 6). 4-Methyl-2,6-di(thiophen-2-yl)selenopyrylium hexafluorophosphate (50.0 mg, 0.107 mmol), 2,6-bis(thiophen-2-yl)-4H-selenopyran-4-one (37.9 mg, 0.118 mmol) and Ac₂O (2.0 mL) were heated for 5 min at 105° C. prior to cooling to ambient temperature, diluting with CH₃CN (3.0 mL) and adding ether to precipitate the product. Yielded 77.6 mg (94%) of a copper bronze solid, mp 233-234° C.: ¹H NMR [500 MHz, CD₂Cl₂] δ 7.92 (br s, 4H), 7.79 (d, 4H, J=5.0 Hz), 7.74 (d, 4H, J=3.0 Hz), 7.33 (t, 4H, J=4.5 Hz), 6.82 (s, 1H); Anal. Calcd for C₂₇H₁₇S₄Se₂.PF₆: C, 41.98; H, 2.22. Found: C, 41.69; H, 2.15; HRMS (ESI) m/z 628.8533 (calcd for C₂₂H₁₇S₄⁸⁰Se₂⁺: 628.8543); λₘₐₓ (CH₂Cl₂)=723 nm, ε=1.3×10⁵ M⁻¹ cm⁻¹; 506 nm, ε=2.9×10⁴ M⁻¹ cm⁻¹.

Synthesis of 4-((2,6-diphenyl-4H-thiopyran-4-ylidene)methyl)-2,6-di(selenophen-2-yl)thiopyrylium hexafluorophosphate (Dye 7). 4-Methyl-2,6-diphenylthiopyrylium hexafluorophosphate (50.0 mg, 0.123 mmol), 2,6-di(selenophen-2-yl)-4H-thiopyran-4-one (50.1 mg, 0.135 mmol) and Ac₂O (2.0 mL) were heated for 5 min at 105° C. prior to cooling to ambient temperature, diluting with CH₃CN (3.0 mL) and adding ether to precipitate the product to yield 79.0 mg (85%) of a copper bronze solid, mp >260° C.: ¹H NMR [500 MHz, CD₂Cl₂] δ 8.44 (d, 2H, J=5.0 Hz), 8.03 (br s, 2H), 7.91 (d, 2H, J=4.0 Hz), 7.83-7.82 (m, 6H), 7.69-7.62 (m, 6H), 7.51 (t, 2H, J=5.0 Hz), 6.72 (s, 1H); Anal. Calcd for C₃₁H₂₁S₂Se₂.PF₆: C, 48.96; H, 2.78. Found: C, 49.09; H, 2.98; HRMS (ESI) m/z 616.9397 (calcd for C₃₁H₂₁S₂⁸⁰Se₂⁺: 616.9410); λₘₐₓ (CH₂Cl₂)=659 nm, ε=1.4×10⁵ M⁻¹ cm⁻¹; 484 nm, ε=1.8×10⁴ M⁻¹ cm¹; 428 nm ε=1.7×10⁴ M⁻¹ cm¹.

Synthesis of 4-((2,6-di(selenophen-2-yl)-4H-thiopyran-4-ylidene)methyl)-2,6-di(selenophen-2-yl)thiopyrylium hexafluorophosphate (Dye 8). 4-Methyl-2,6-di(selenophen-2-yl)thiopyrylium hexafluorophosphate (50.0 mg, 96.9 μmol), 2,6-di(selenophen-2-yl)-4H-thiopyran-4-one (39.6 mg, 0.107 mmol) and Ac₂O (2.0 mL) were heated for 5 min at 105° C. prior to cooling to ambient temperature, diluting with CH₃CN (3.0 mL) and adding ether to precipitate the product to yield 64.2 mg (76%) of a copper bronze solid, mp >260° C.: ¹H NMR [500 MHz, CD₃CN] δ 8.48 (d, 4H, J=6.0 Hz), 7.93 (d, 4H, J=3.5 Hz), 7.74 (s, 4H), 7.47 (t, 4H, J=5.0 Hz), 6.57 (s, 1H); Anal. Calcd for C₂₇H₁₇S₂Se₄.PF₆: C, 37.43; H, 1.98. Found: C, 37.70; H, 2.06; HRMS (ESI) m/z 724.7393 (calcd for C₂₂H₁₇S₂⁸⁰Se₄⁺: 724.7427); λₘₐₓ (CH₂Cl₂)=687 nm, ε=1.1×10⁵ M⁻¹ cm⁻¹; 491 nm, ε=2.8×10⁴ M⁻¹ cm⁻¹.

Synthesis of 4-(3-(2,6-diphenyl-4H-selenopyran-4-ylidene)prop-1-enyl)-2,6-diphenylselenopyrylium hexafluorophosphate (CAS Registry Number: 51848-65-8) (Dye 9). 4-Methyl-2,6-di(phenyl)selenopyrylium hexafluorophosphate (0.190 g, 0.417 mmol), 4-(2,6-diphenyl-4H-selenopyran-4ylidene)acetaldehyde (0.155 g, 0.459 mmol) and Ac₂O (3.0 mL) were combined in a round bottom flask and heated at 105° C. for 10 min. The reaction was cooled to ambient temperature, precipitated with ether, and the collected solid recrystallized from CH₃CN/ether to yield 0.278 g (86%) of a golden-green solid: ¹H NMR [500 MHz, CD₂Cl₂] δ 8.59 (t, 1H, J=13.5 Hz), 8.40-7.80 (br s, 4H), 7.71 (d, 8H, J=7.0 Hz), 7.63-7.59 (m, 12H), 6.85 (d, 2H, J=13.0 Hz); Anal. Calcd for C₃₇H₂₇Se₂.PF₆: C, 57.38; H, 3.51; F, 14.72. Found: C, 57.34; H, 3.48; F, 14.76; LRMS (ESI) m/z 631.2 (calcd for C₃₂H₂₂⁸⁰Se₂: 631.0); λₘₐₓ (CH₂Cl₂)=806 nm, ε=2.5×10⁵ M⁻¹ cm⁻¹.

Synthesis of 4-(3-(2,6-diphenyl-4H-thiopyran-4-ylidene)prop-1-enyl)-2,6-diphenylselenopyrylium hexafluorophosphate (CAS Registry Number: 79054-92-5) (Dye 10). 4-Methyl-2,6-di(phenyl)thiopyrylium hexafluorophosphate (0.128 g, 0.312 mmol), 4-(2,6-diphenyl-4H-selenopyran-4ylidene)acetaldehyde (0.157 g, 0.344 mmol) and Ac₂O (2.0 mL) were combined in a round bottom flask and heated at 105° C. for 10 min. The reaction was cooled to ambient temperature, CH₃CN (2.0 mL) was added and ether was used to precipitate product from solution to yield 0.196 g (86%) of a copper-bronze solid: ¹H NMR [500 MHz, CD₂Cl₂] δ 8.54 (t, 1H, J=13.0 Hz), 8.20-7.80 (br s, 4H), 7.78 (d, 4H, J=8.0 Hz), 7.70 (d, 4H, J=7.5 Hz), 7.66-7.58 (m, 12H), 6.78 (d, 2H, J=13.5 Hz); Anal. Calcd for C₃₉H₃₄O₃Se₂.PF₆: C, 61.08; H, 3.74. Found: C, 61.10; H, 3.68; LRMS (ESI) m/z 583.3 (calcd for C₃₇H₂₇S⁸⁰Se: 583.1); λₘₐₓ (CH₂Cl₂)=784 nm, ε=2.0×10⁵ M⁻¹ cm⁻¹.

Synthesis of 4-(3-(2,6-dithiophen-2-yl-4H-thiopyran-4-ylidene)prop-1-enyl)-2,6-diphenylselenopyrylium hexafluorophosphate (Dye 11). 4-Methyl-2,6-di(phenyl)selenopyrylium hexafluorophosphate (0.102 g, 0.225 mmol), 4-(2,6-(thiophene-2-yl)-4H-thiopyran-4ylidene)acetaldehyde (75.0 mg, 0.248 mmol) and Ac₂O (3.0 mL) were combined in a round bottom flask and heated at 105° C. for 5 min. The reaction was cooled to ambient temperature, precipitated with ether, and the collected solid recrystallized from CH₃CN/ether to yield 0.145 g (87%) of a bronze solid, mp 229-231° C.: ¹H NMR [500 MHz, CD₂Cl₂] δ 8.46 (t, 1H, J=13.0 Hz), 7.71-7.58 (m, 18H), 7.26 (t, 2H, J=4.0 Hz), 6.77 (d, 1H, J=13.0 Hz), 6.70 (d, 1H, J=14.0 Hz); Anal. Calcd for C₃₃H₂₃S₃Se.PF₆: C, 53.59; H, 3.13; F, 15.41. Found: C, 53.79; H, 3.13; F, 15.19; HRMS (ESI) m/z 595.0125 (calcd for C₃₃H₂₃S₃⁸⁰Se⁺: 595.0122); λₘₐₓ (CH₂Cl₂)=810 nm, ε=2.5×10⁵ M⁻¹ cm⁻¹.

Synthesis of 4-(3-(2,6-dithiophen-2-yl-4H-thiopyran-4-ylidene)prop-1-enyl)-2,6-diphenylthiopyrylium hexafluorophosphate (Dye 12). 4-Methyl-2,6-diphenylthiopyrylium hexafluorophosphate (30.0 mg, 73.0 μmol), 4-(2,6-(thiophene-2-yl)-4H-thioopyran-4ylidene)acetaldehyde (24.4 mg, 81.0 μmol) and Ac₂O (1.0 mL) were combined in a round bottom flask and heated at 105° C. for 5 min. The reaction was cooled to ambient temperature, CH₃CN (4.0 mL) was added and ether was used to precipitate product from solution to yield 45.0 mg (88%) of a bronze solid, mp >260° C.: ¹H NMR [500 MHz, CD₂Cl₂] δ 8.44 (t, 1H, J=13.0 Hz), 8.40-7.80 (br s, 4H), 7.78 (d, 4H, J=7.0 Hz), 7.67-7.59 (m, 10H), 7.24 (t, 2H, J=4.5 Hz), 6.71 (d, 1H, J=13.0 Hz), 6.63 (d, 1H, J=13.5 Hz); Anal. Calcd for C₃₃H₂₃S₄.PF₆: C, 57.21; H, 3.35. Found: C, 56.97; H, 3.36; HRMS (ESI) m/z 547.0674 (calcd for C₃₃H₂₃S₄⁺: 547.0677); λₘₐₓ (CH₂Cl₂)=789 nm, ε=2.2×10⁵ M⁻¹ cm⁻¹.

Synthesis of 4-(3-(2,6-dithiophen-2-yl-4H-thiopyran-4-ylidene)prop-1-enyl)-(2,6-dithiophen-2-yl)thiopyrylium hexafluorophosphate (CAS Registry Number: 95410-36-9) (Dye 13). 4-Methyl-2,6-di(thiophen-2-yl)thiopyrylium hexafluorophosphate (11.0 mg, 26.2 μmol), 4-(2,6-(thiophene-2-yl)-4H-thioopyran-4ylidene)acetaldehyde (9.5 mg, 31.4 μmol) and Ac₂O (1.0 mL) were combined in a round bottom flask and heated at 105° C. for 5 min. The reaction was cooled to ambient temperature, CH₂Cl₂ (2.0 mL) was added and ether was used to precipitate product from solution to yield 17.8 mg (94%) of a bronze solid, mp >260° C.: ¹H NMR [500 MHz, CD₃CN] δ 8.32 (t, 1H, J=13.5 Hz), 7.68 (d, 2H, J=4 Hz), 7.56 (br s, 4H) 7.14 (t, 4H, J=4.5 Hz), 6.48 (d, 2H, J=13.0 Hz); Anal. Calcd for C₂₉H₁₉S₆.PF₆: C, 49.42; H, 2.72. Found: C, 49.19; H, 2.79; HRMS (ESI) m/z 558.9805 (calcd for C₂₉H₁₉S₆⁺: 558.9806); λₘₐₓ (CH₂Cl₂)=813 nm, ε=2.8×10⁵ M⁻¹ cm⁻¹.

Synthesis of 4-(3-(2,6-di(selenophen-2-yl)-4H-thiopyran-4-ylidene)prop-1-en-1-yl)-2,6-di(selenophen-2-yl)thiopyrylium hexafluorophosphate (Dye 14). 4-Methyl-2,6-di(selenophen-2-yl)thiopyrylium hexafluorophosphate (47.1 mg, 91.4 μmol), 2-((2,6-di(selenophen-2-yl)-4H-thiopyran-4-ylidene)acetaldehyde (47.1 mg, 0.101 mmol) and Ac₂O (2.0 mL) were heated for 5 min at 105° C. prior to cooling to ambient temperature, diluting with CH₃CN (3.0 mL) and adding ether to precipitate the product to yield 71.0 mg (87%) of a copper bronze solid, mp 249-251° C.: ¹H NMR [500 MHz, CD₃CN] δ 8.51-8.46 (m, 5H), 7.88 (d, 4H, J=3.0 Hz), 7.71 (br s, 4H), 7.46 (t, 4H, J=4.5 Hz), 6.62 (d, 2H, J=13.0 Hz); Anal. Calcd for C₂₉H₁₉S₂Se₄.PF₆: C, 39.03; H, 2.15. Found: C, 39.28; H, 2.19; HRMS (ESI) m/z 750.7560 (calcd for C₂₉H₁₉S₂⁸⁰Se₄⁺: 750.7584); λₘₐₓ (CH₂Cl₂)=826 nm, ε=2.3×10⁵ M⁻¹ cm⁻¹; 750 nm, ε=5.2×10⁴ M⁻¹ cm⁻¹; 490 nm, ε=2.8×10⁴ M⁻¹ cm⁻¹.

Synthesis of 4-((1E,3E)-5-(2,6-di(thiophen-2-yl)-4H-thiopyran-4-ylidene)penta-1,3-dien-1-yl)-2,6-di(thiophen-2-yl)thiopyrylium (Dye 18). 4-Methyl-2,6-di(thiophen-2-yl)thiopyrylium hexafluorophosphate (0.200 g, 0.476 mmol), N-(2-(phenylamino)ethen-1yl)methylenebenzaminium hexafluorophosphate (25, 87.6 mg, 0.238 mmol), NaOAc (39.1 mg, 0.476 mmol), AcOH (1.0 mL) and Ac₂O (1.0 mL) were combined and heated at 90° C. for 15 min. The mixture was cooled to ambient temperature and diluted with H₂O (50 mL). The product was extracted with a mixture of CH₂Cl₂ (3×20 mL) and CH₃CN (3×5 mL). The organic layer was dried with Na₂SO₄, concentrated and the product recrystallized from hot CH₃CN and an equivalent amount of ether to yield 82.9 mg (48%) of a red, metallic solid. λₘₐₓ (CH₂Cl₂)=944 nm.

Synthesis of 4-((1E,3E)-5-(2,6-di(selenophen-2-yl)-4H-selenopyran-4-ylidene)penta-1,3-dien-1-yl)-2,6-di(selenophen-2-yl)selenopyrylium (Dye 19). 4-Methyl-2,6-di(selenophen-2-yl)selenopyrylium hexafluorophosphate (28, 0.150 g, 0.266 mmol), N-(2-(phenylamino)ethen-1yl)methylenebenzaminium hexafluorophosphate (25, 49.0 mg, 0.133 mmol), NaOAc (21.8 mg, 0.266 mmol), AcOH (1.0 mL) and Ac₂O (1.0 mL) were combined and heated at 90° C. for 15 min. The mixture was cooled to ambient temperature and diluted with H₂O (50 mL). The product was extracted with a mixture of CH₂Cl₂ (3×20 mL) and CH₃CN (3×5 mL). The organic layer was dried with Na₂SO₄, concentrated and the crude product gave λₘₐₓ (CH₂Cl₂)=1001 nm.

Synthesis of 4-((E)-2-((E)-2-chloro-3-(2-(2,6-diphenyl-4H-thiopyran-4-ylidene)ethylidene)cyclohex-1-en-1-yl)vinyl)-2,6-diphenylthiopyrylium hexafluorophosphate (Dye 20). 4-Methyl-2,6-diphenylselenopyrylium (0.100 g, 0.245 mmol), N-((E)-((E)-2-chloro-3-((phenylamino)methylene)cyclohex-1-en-1-yl)methylene)benzenaminium hexafluorophosphate (26, 43.9 mg, 0.123 mmol) and NaOAc (20.1 mg, 0.245 mmol) were stirred in a mixture of Ac₂O (1.0 mL) and AcOH (1.0 mL). This mixture was heated at 95° C. for 1 h prior to cooling to ambient temperature and stirring with 10% aqueous HPF₆ (30 mL) for 1 h. The product was extracted in CH₂Cl₂ (50 mL), the organic layer dried with Na₂SO₄, and after concentration purified on SiO₂ with a 20% EtOAc/CH₂Cl₂ eluent. Fractions containing product were combined and recrystallized from CH₃CN/ether to yield 39.8 mg (40%) of a copper bronze solid, mp 200-202° C.: ¹H NMR [500 MHz, CD₃CN] δ 8.34 (d, 2H, J=14.5 Hz), 7.80 (br s, 4H), 7.77-7.75 (m, 8H), 7.62-7.57 (m, 12H), 6.71 (d, 2H, J=14.5 Hz), 2.77 (t, 4H, J=6.5 Hz), 1.98-1.96 (m, 2H); λₘₐₓ (CH₂Cl₂)=1042 nm, ε=1.0×10⁵ M⁻¹ cm⁻¹.

Synthesis of 4-((E)-2-((E)-2-chloro-3-(2-(2,6-di(thiophen-2-yl)-4H-thiopyran-4-ylidene)ethylidene)-cyclohex-1-en-1-yl)vinyl)-2,6-di(thiophen-2-yl)thiopyrylium hexafluorophosphate (Dye 21). 4-Methyl-2,6-di(thiophen-2-yl)thiopyrylium hexafluorophosphate (0.200 g, 0.476 mmol), N-((E)-((E)-2-chloro-3-((phenylamino)methylene)cyclohex-1-en-1-yl)methylene)benzenaminium chloride (26, 85.3 mg, 0.238 mmol), NaOAc (39.1 mg, 0.476 mmol), AcOH (1.0 mL) and Ac₂O (1.0 mL) were combined and heated at 95° C. for 15 min. The mixture was cooled to ambient temperature and diluted with H₂O (50 mL). The product was extracted with a mixture of CH₂Cl₂ (3×20 mL) and CH₃CN (3×5 mL). The organic layer was dried with Na₂SO₄, and concentrated and the crude product gave λₘₐₓ (CH₂Cl₂)=1119 nm.

Synthesis of Benzopyrylium Derivatives. S3 and S9 were prepared in a similar manner to literature procedures (J Org. Chem. 1980, 45, 4611-15), but S3 is a novel derivative of this class of compounds. S4 and S6 were prepared in accordance with literature procedures (Organometallics 1988, 7, 1131-1147; J. Org. Chem. 1982, 47, 5235-5239; J Org Chem 2003, 68 (5), 1804-1809; Chem. Heterocycl. Compd. (N.Y.) 1998, 34, 438-443; J. Org. Chem. 1980, 45, 4611-4615). The reaction in Ac₂O to form the final dye compounds S5, S8, and S10 follows literature precedent as well (J. Org. Chem. 1982, 47, 5235-5239).

### Preparation of (Z)-3-(thiophene-2-yl)-3-(phenylselanyl)acrylic acid

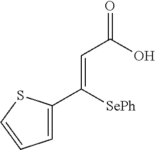

In a round bottom flask under argon, diphenyl diselenide (0.734 g, 2.35 mmol) was dissolved in THF (5 mL) and NaBH₄ (0.356 g, 9.41 mmol) was added in one portion. EtOH (10 mL) was added over a 10 min period until bubble formation had ceased and the solution was colorless. Ethyl 3-(thiophene-2-yl)propiolate (0.782 g, 4.71 mmol) was dissolved in THF (5 mL) and added to the reduced selenide, after which the solution was heated to reflux over a 10 min period and then 3 M aqueous KOH (20 mL) was added and the reaction refluxed overnight. After cooling to rt 3 M aqueous HCl was added to bring the solution to a pH≈1, the product extracted with EtOAc (50 mL), the organic layer dried with MgSO₄ and after concentration recrystallized from EtOAc/hexanes to yield 0.939 g (65%) of an off-white solid. NMR revealed only trace (<5%) of the E isomer, mp 139.0-140.5° C.: ¹H MNR [500 MHz, CDCl₃] δ 7.35 (d, 2H, J=7.5 Hz), 7.21-7.17 (m, 1H), 7.15-7.12 (m, 3H), 6.76-6.71 (m, 2H), 6.54 (s, 1H); ¹³C NMR [75.5 MHz, CDCl₃] δ 171.05, 154.03, 140.49, 136.31, 135.26, 130.02, 129.90, 129.55, 128.76, 128.07, 127.23, 126.88, 117.42, 115.13; HRMS (EI) m/z 309.9569 (calcd for C₁₃H₁₀O₂S⁸⁰Se: 309.9561).

### Preparation of 2-(thiophene-2-yl)benzoselenopyran-4-one

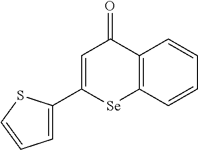 [SMILES: O=c1cc(-c2cccs2)[se]c2ccccc12]

Methanesulfonic acid (6.76 mL) and P₂O₅ (1.00 g, 3.52 mmol) were placed in a flame dried flask under argon and heated to 65° C. until all of the P₂O₅ was dissolved. (Z)-3-(thiophene-2-yl)-3-(phenylselanyl)acrylic acid (0.400 g, 1.29 mmol) was added in portions over approximately 5 min, and then allowed to stir at 65° C. for 5 min. The reaction was then quenched by pouring into satd. aqueous NaHCO₃ (250 mL), and the product extracted with (3×75 mL) of CH₂Cl₂. The organic layer was dried with MgSO₄, concentrated under reduced pressure and then purified on SiO₂ with a 10% EtOAc/CH₂Cl₂ eluent to yield 0.154 g (41%) of a light brown solid, mp 130-132° C.: ¹H MNR [500 MHz, CDCl₃] δ 8.58 (dxd, 1H, J=8.0, 2.0 Hz), 7.66 (d, 1H, J=8.0 Hz), 7.56-7.50 (m, 4H), 7.39 (s, 1H), 7.16 (t, 1H, J=4 Hz); ¹³C NMR [75.5 MHz, CDCl₃] δ 182.53, 144.96, 140.72, 135.81, 131.86, 131.69, 129.98, 129.05, 128.42, 128.04, 127.79, 127.29, 123.55; HRMS (EI) m/z 291.9465 (calcd for C₁₃H₈OS⁸⁰Se: 291.9456).

### Preparation of 4-methyl-2-(thiophene-2-yl)selenobenzopyrylium hexafluorophosphate

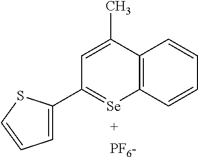

2-(Thiophene-2-yl)benzoselenopyran-4-one (0.150 g, 0.515 mmol) was dissolved in anhydrous THF (5.0 mL) in a flame dried flask under argon. 3.0 M MeMgBr (0.520 mL, 1.56 mmol) was added slowly and the reaction allowed to stir at rt for ½ h. This was then poured into 10% aqueous HPF₆, stirred for ½h, and the product isolated by filtration. The solid was dissolved in CH₂Cl₂, dried with Na₂SO₄, and after concentration recrystallized from CH₃CN/ether to yield 0.171 g (76%) of a bright orange solid, mp 137-140° C.: ¹H MNR [500 MHz, CD₃CN] 8.82-8.79 (m, 1H), 8.57 (s, 1H), 8.51-8.49 (m, 1H), 8.35-8.33 (m, 2H), 8.05-8.03 (m, 2H), 7.49 (t, 1H, J=4.5 Hz), 3.07 (s, 3H); ¹³C NMR [75.5 MHz, CD₃CN] δ 178.08, 167.28, 145.16, 142.39, 141.82, 136.60, 134.95, 133.17, 132.80, 132.11, 130.81, 130.40, 25.77; HRMS (EI) m/z 289.9663 (calcd for C₁₄H₁₀S⁸⁰Se: 289.9663). (Phys. Med. Biol. 1994, 39, 1705-1720)

### Preparation of 2-(2-phenyl)-4H-selenobenzopyran-4-ylidene)acetaldehyde

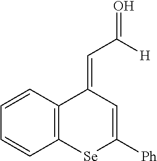 [SMILES: O=C/C=C1\C=C(c2ccccc2)[Se]c2ccccc21]

4-Methyl-2-phenylselenobenzopyrylium hexafluorophosphate (0.100 g, 0.233 mmol), N,N-Dimethylthioformamide (59.4 μL, 0.698 mmol) and Ac₂O (2.0 mL) were combined in a small round bottom flask and heated at 95° C. for 1 h. After cooling to rt the solution was diluted with ether. The formed iminium salt was allowed to precipitate in the freezer overnight, and then isolated by filtration to yield a bright orange solid. This solid was dissolved in CH₃CN (3.0 mL) and satd. aqueous NaHCO₃ (3.0 mL) was added. This mixture was heated to 80° C. over 15 min, and kept at that temperature for ½ h. After diluting with H₂O (30 mL) the product was extracted with CH₂Cl₂ (3×20 mL), dried with Na₂SO₄ and purified on SiO₂ with a CH₂Cl₂ eluent (R_{f}=0.56) to give a yellow oil that was recrystallized in CH₂Cl₂/hexanes to yield 66.0 mg (91%) of a yellow crystalline solid, mp 89-92° C.: ¹H MNR [300 MHz, CDCl₃] δ 10.35 (d, 1H, J=7.0 Hz), 8.34 (s, 1H), 8.00-7.97 (m, 1H), 7.65-7.57 (m, 3H), 7.49-7.40 (m, 5H) 6.53 (d, 1H, J=6.5 Hz); ¹³C NMR [75.5 MHz, CDCl₃] δ 189.79, 148.35, 143.18, 138.86, 132.24, 129.88, 129.69, 129.12, 128.08, 127.12, 126.86, 119.35, 119.06; HRMS (EI) m/z (calcd for C₁₇H₁₂O⁸⁰Se:).

### Preparation of 2-phenyl-4-((2-phenyl-4H selenobenzopyran-4-ylidene)methyl)selenobenzopyrylium hexafluorophosphate (CAS Registry Number: 47732-21-8)

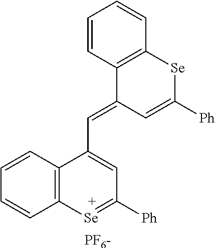

4-methyl-2-phenylselenobenzopyrylium hexafluorophosphate (0.200 g, 0.466 mmol), 2-phenylbenzoselenopyran-4-one (0.146 g, 0.513 mmol), and Ac₂O (4.0 mL) were combined and heated at 105° C. for 10 min. The solution was diluted with CH₃CN (4.0 mL) and precipitated with ether. The resulting solid was recrystallized from CHCl₃/ether to yield 0.237 g (73%) of a dark green solid, mp 154-156° C.: ¹H MNR [500 MHz, CD₂Cl₂] δ 8.73-8.72 (m, 2H), 8.57 (br. s, 2H), 8.14-8.12 (m, 3H), 7.85-7.83 (m, 4H), 7.64-7.60 (m, 6H), 7.50 (t, 4H, J=8.0 Hz); Anal. Calcd for C₃₁H₂₁Se₂.PF₆: C, 53.47; H, 3.04. Found: C, 53.40; H, 3.06; HRMS (ESI) m/z 552.9966 (calcd for C₃₁H₂₁⁸⁰Se₂: 552.9968); λₘₐₓ (CH₂Cl₂)=748 nm, ε=7.6×10⁴ M⁻¹ cm⁻¹.

### Preparation of 2-(thiophene-2-yl)-4-((2-(thiophen-2-yl)-4H selenobenzopyran-4-ylidene)methyl)selenobenzopyrylium hexafluorophosphate (Dye 17)

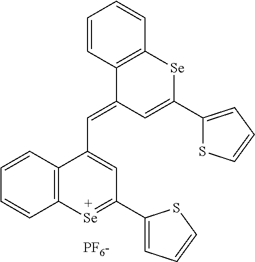

4-Methyl-2-(thiophene-2yl)selenobenzopyrylium hexafluorophosphate (30.0 mg, 68.9 μmol), 2-(thiophene-2-yl)benzoselenopyran-4-one (22.1 mg, 75.8 μmol), and Ac₂O (1.0 mL) were combined and heated at 105° C. for 10 min. The solution was diluted with CH₃CN (3.0 mL) and precipitated with ether to yield 40.9 mg (84%) of a bronze solid, mp 199-201° C.: ¹H MNR [500 MHz, CD₂Cl₂] δ; Anal. Calcd for C₂₇H₁₇S₂Se₂.PF₆: C, 45.78; H, 2.42. Found: C, 45.65; H, 2.63; HRMS (ESI) m/z 564.9105 (calcd for C₂₂H₁₂S₂⁸⁰Se₂: 564.9097); λₘₐₓ (CH₂Cl₂)=786 nm, ε=7.8×10⁴ M⁻¹ cm⁻¹.

### Synthesis of 2-phenyl-4-((E)-3-((E)-2-phenyl-4H-thiochromen-4-ylidene)prop-1-en-1-yl)thiochromenylium hexafluorophosphate (Dye 15)

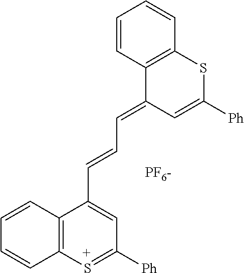

4-Nethyl-2-phenylthiochromenylium hexafluorophosphate (65.1 mg, 0.170 mmol), (E)-2-(2-phenyl-4H-thiochromen-4-ylidene)acetaldehyde (54.0 mg, 0.204 mmol), and Ac₂O (2.0 mL) were combined and heated at 105° C. for 10 min. The solution was cooled to rt, diluted with CH₃CN, and the product precipitated with ether to yield 94.4 mg (88%) of a copper bronze solid, mp 253-254° C.: ¹H NMR [500 MHz, CD₃CN] δ Due to poor solubility in common NMR solvents resolution was extremely poor; Anal. Calcd for C₃₃H₂₃S₂.PF₆: C, 63.05; H, 3.69. Found: C, 62.79; H, 3.88; LRMS (ESI) m/z 483.4 (calcd for C₃₃H₂₃S₂⁺: 483.1); λₘₐₓ (CH₃CN)=789 nm, ε=1.5×10⁵ M⁻¹ cm⁻¹.

### Synthesis of 4-(3-(2,6-diphenyl-4H-thiopyran-4-ylidene)prop-1-enyl)-2-phenylselenobenzopyrylium hexafluorophosphate (Dye 16)

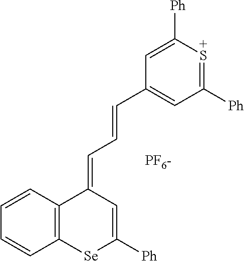

4-Methyl-2,6-di(phenyl)thiopyrylium hexafluorophosphate (51.5 mg, 0.126 mmol), 2-phenylbenzoselenopyran-4-one (43.2 mg, 0.139 mmol), and Ac₂O (2.0 mL) were combined and heated at 105° C. for 10 min. The solution was diluted with CH₃CN (3.0 mL) and precipitated with ether to yield 65.3 mg (74%) of a copper bronze solid, mp >260° C.: ¹H NMR [500 MHz, CD₃CN] Spectrum was unresolved due to poor solubility and solution dynamics of this compound. Anal. Calcd for C₃₅H₂₅SSe.PF₆: C, 59.92; H, 3.59. Found: C, 59.74; H, 3.48; HRMS (ESI) m/z 557.0853 (calcd for C₃₅H₂₅S⁸⁰Se: 557.0837); λₘₐₓ (CH₃CN)=748 nm, ε=6.1×10⁴ M⁻¹ cm⁻¹.

### Example 2

In this example, we describe the design and synthesis of a novel group of near infrared absorbing 2-thienyl-substituted chalcogenopyrylium dyes tailored to have high affinity for gold. When adsorbed onto gold nanoparticles, these dyes produce biocompatible SERRS-nanoprobes with attomolar limits of detection amenable to ultrasensitive in vivo multiplexed tumor and disease marker detection.

One notable feature of the pyrylium dyes is the ease in which a broad range of absorptivities can be accessed, and consequently be matched with the NIR light source by careful tuning of the dye's optical properties. Specifically, the large differences in absorption maxima introduced by switching the chalcogen atom is a useful property of this dye class. nother important consideration is the affinity of the reporter for the surface of gold. Since the SERS effect decreases exponentially as a function of distance from the nanoparticle, it is important that the Raman reporter is near the gold surface. The 2-thienyl substituent provides a novel attachment point to gold for Raman reporters. The 2-thienyl group is not only part of the dye chromophore, but also can be rigorously coplanar with the rest of the chromophore. This allows the dye molecules to be in close proximity to the nanoparticle surface, creating a more intense SERRS-signal.

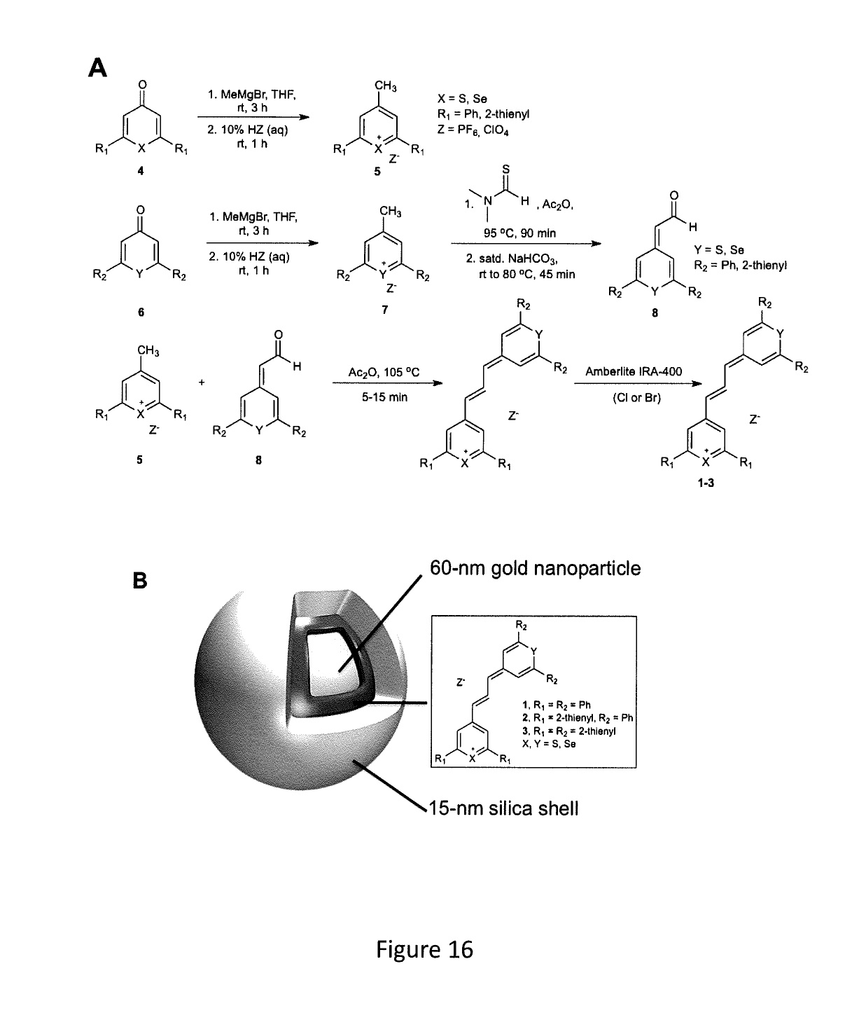

Results. Chalcogenopyrylium dye synthesis and characterization. Cationic chalcogenopyrylium dyes 1-3, with absorption maxima near the 785-nm emission of the detection laser were synthesized as outlined in FIG. 16 A. The addition of MeMgBr to the known chalcogenopyranones (4, 6), followed by dehydration with the appropriate acid (HZ), yields 4-methyl pyrylium compounds (5, 7) with the desired counterion (PF₆⁻ or ClO₄⁻). The condensation of 7 with N,N-dimethylthioformamide in Ac₂O, and subsequent hydrolysis of the intermediate iminium salt yields the (chalcogenopyranylidene) acetaldehyde 8, the penultimate compound leading to trimethine chalcogenopyrylium dyes. Condensation of 4-methylpyrylium salt 5 and the (chalcogenopyranylidene)acetaldehyde 8 bearing the desired R groups and chalcogen atom in hot Ac₂O forms the final dye compounds 1-3 that are substituted with 2-phenyl or 2-thienyl groups, and different combinations of chalcogen atoms (S or Se) (Table 2). The CL and Br counterions of dye 1a were accessed by treating the PF₆ salts with an Amberlite® ion exchange resin. Full synthetic details including yields and characterization are available in the Supporting Information.

TABLE 2
Chalcogenopyrylium dye structural and optical characteristics

| Dye | X | Y | λₘₐₓ (CH₂Cl₂) | Log (ε) | Yield (%) |
|---|---|---|---|---|---|
| 1a | Se | Se | 806 nm | 5.40 | 86 |
| 1b | S | Se | 784 nm | 5.30 | 86 |
| 2a | S | Se | 810 nm | 5.40 | 87 |
| 2b | S | S | 789 nm | 5.34 | 88 |
| 3 | S | S | 813 nm | 5.45 | 94 |

SERRS-nanoprobe synthesis and characterization. Chalcogenopyrylium dyes 1-3 were dissolved in dry N,N-dimethylformamide (DMF), at a concentration between 1.0 and 10 mM, and were subsequently used to generate the SERRS-nanoprobes. The SERRS-nanoprobes consist of a gold core onto which the SERRS-reporter is adsorbed, which is then protected by an encapsulating silica layer (FIG. 16 B, Table 2). The pyrylium based SERRS-nanoprobes were synthesized by encapsulating 60-nm spherical citrate-capped gold nanoparticles via a modified Stöber procedure in the presence of the reporter. After 25 minutes, the reaction was quenched by the addition of ethanol and the SERRS-nanoprobes were collected through centrifugation. Typically, the as-synthesized SERRS-nanoprobes had a mean diameter of ˜100 nm.

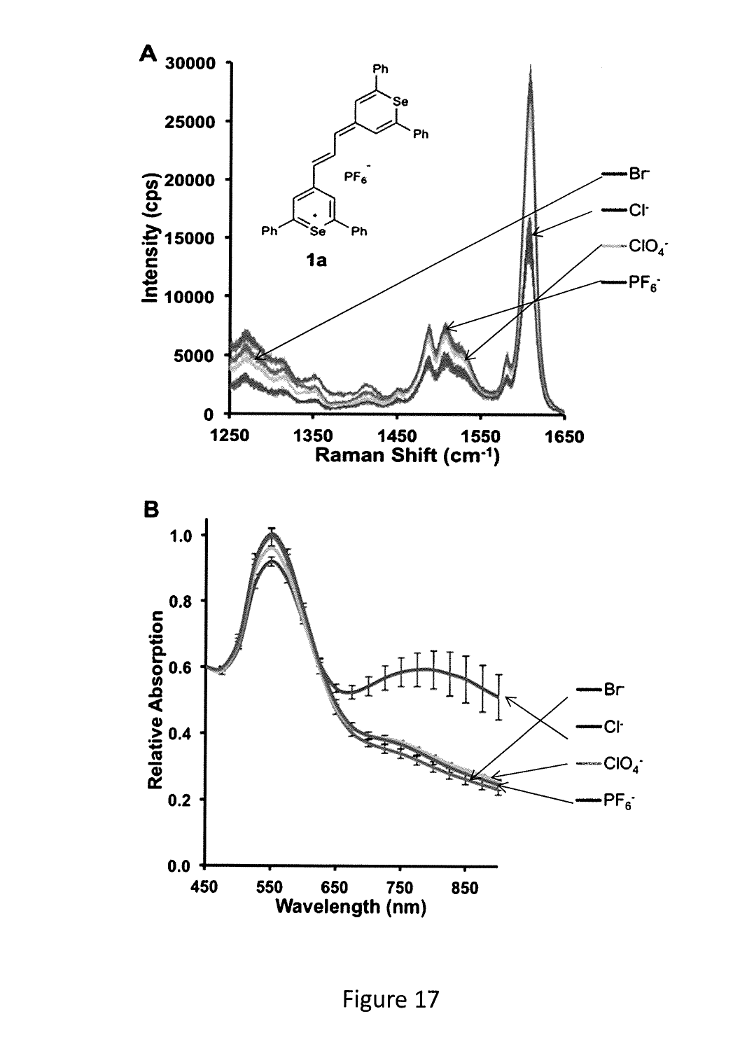

Effect of counterion on colloidal stability and SERRS-signal. In previous reports, the dye counterion was shown to affect the structural and electronic properties of polymethine dyes and the solubility of chalcogenopyrylium dyes. Since SERRS is highly dependent on these factors, we evaluated the effect of the counterion (Z⁻) on the SERRS spectrum, intensity, and colloidal stability of the pyrylium-based SERRS-nanoprobes. We compared chloride (Cl⁻), bromide (Br⁻), perchlorate (ClO₄⁻), and hexafluorophosphate (PF₆⁻) as counterions for chalcogenopyrylium dye 1a. The SERRS-nanoprobes were synthesized in the presence of equimolar amounts (10 μM) of CP dye 1a.Z⁻ (where Z⁻═Cl⁻, Br⁻, ClO₄⁻, or PF₆⁻). The counterion introduces almost no difference in optical properties (e.g. absorption maxima, extinction coefficient). Furthermore, with the exception of the chloride counter-ion, the Raman shifts and intensity of 1a were minimally affected by the different counterions (FIG. 17 B). The colloidal stability, however, was shown to be highly counterion dependent (FIG. 17 B). The least chaotropic counterion, Cl⁻, strongly destabilized the gold colloids and caused aggregation for SERRS-nanoprobes utilizing 1a as a reporter as evidenced by the strong absorption between 700-900 nm. The strongest chaotropic anion, PF₆⁻, did not affect colloidal stability during the synthesis of SERRS-nanoprobes as evidenced by the strong absorption at 540 nm and low absorbance between 700-900 nm (monomeric 60-nm spherical gold nanoparticles have an absorption maximum around 540 nm). Since the PF₆⁻ anion induced the least nanoparticle aggregation, it was used for further SERRS experiments.

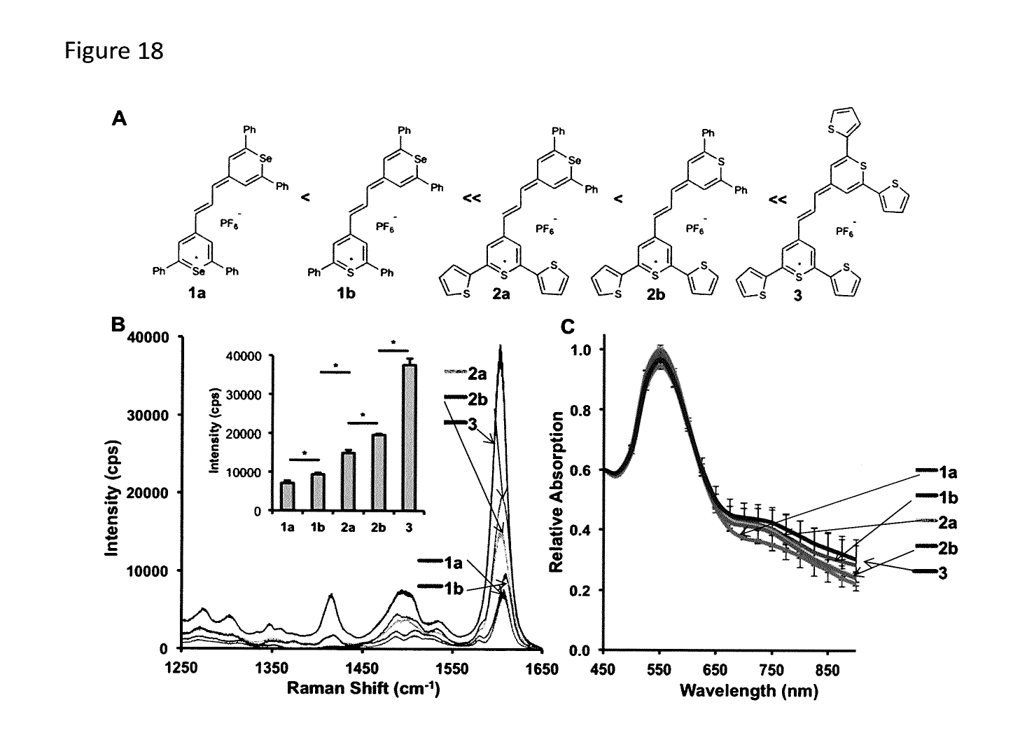

Effect of increased affinity on colloidal stability and SERRS-signal. We also examined the SERRS-signal intensity as a function of the number of sulfur atoms in the dye. Sulfur-containing functionality has been used frequently to adhere molecules to gold, with several reports using thiol or lipoic acid functional groups to add sulfur-containing functionality. In our structures, 2-thienyl groups attached to the 2- and 6-positions of the dye were used to bind the dyes to the gold surface. We also explored the impact of the chalcogen atoms in the chalcogenopyrylium core, switching a Se (1a and 2a) to S (1b and 2b). The chalcogen switch was used to increase semi-covalent interactions with the gold surface, and also to create a chromophore that had a more resonant absorption with the 785-nm detection laser (Table 2). Chalcogenopyrylium dyes 1-3 were used at a final concentration of 1.0 μM, which prevented nanoparticle aggregation for dye 3. FIG. 18 A shows the molecular structures of the chalcogenopyrylium dyes. The SERRS intensity of the different as-synthesized pyrylium-based SERRS-nanoprobes, which were synthesized at equimolar reporter concentrations, were measured at equimolar SERRS-nanoprobe concentrations at low laser power to prevent CCD-saturation (50 μW/cm², 1.0-s acquisition time, 5× objective). We specifically focused on the 1600 cm⁻¹ peak, which corresponds to aromatic ring stretching modes; and is a mode shared by chalcogenopyrylium dyes 1-3. The SERRS-signal intensity of the 1600 cm⁻¹ peak increased significantly as the number of 2-thienyl substituents increased (FIG. 18 B) without causing significant aggregation (FIG. 18 C). Thus, 3 produced the highest SERRS-signal, which was significantly more intense than 2a/2b or 1a/1b (P<0.05) and 2a/2b were significantly more intense than 1a/1b (P<0.05). There was a less noticeable, but significant, increase from the chalcogen switch in the core (1a/1b and 2a/2b being significantly different (P<0.05)). This strongly supports the hypothesis that 2-thienyl groups are an effective means of adhering dyes to gold, resulting in brighter SERRS-nanoprobes.

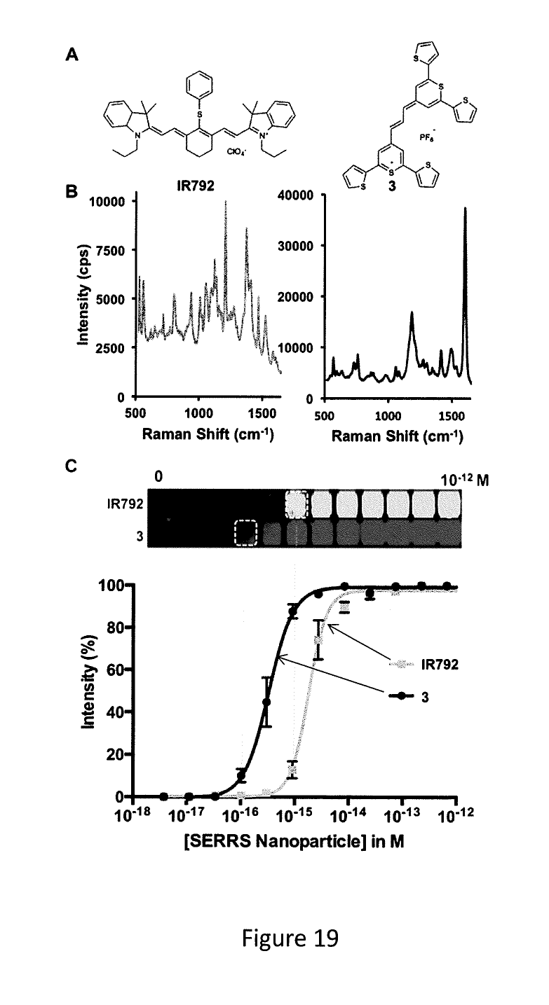

Comparison of CP-dye 3 with a cyanine-based SERRS-reporter. In order to assess the quality of our optimized nanoprobe, thiopyrylium dye 3 and commercially available IR792 (FIG. 19 A), which has been previously used to generate surface-enhanced resonance Raman scattering nanoprobes, were studied. A direct comparison of the nanoprobes synthesized in the presence of equimolar (1.0 μM) amounts of 3 and IR792 shows a 5-6 fold higher signal for nanoprobes generated with dye 3 (FIG. 19 B). It is interesting to note that a fluorescence background is minimal in the SERRS spectra of the CP- and cyanine-based SERRS-nanoprobes. Whereas fluorescence interference would not be expected from chalcogenopyrylium dyes containing heavy chalcogens that enhance intersystem crossing, fluorescence interference could be expected for the cyanine dye IR792. In fact, when equimolar amounts of the CP dyes 1a-3 and IR792 were incorporated in silica (without gold nanoparticle), IR792 demonstrated strong fluorescence when excited at 785-nm (50 μW/cm², 1.0 s acquisition time), while minimal fluorescence was observed for CP 1a-3. As shown in FIG. 19 B, the fluorescence interference of the cyanine dye IR792 is minimal in its SERRS spectrum. This is due to quenching effects near the surface of the nanoparticle.

A concentration series of the as-synthesized SERRS-nanoprobes was generated in triplicate fashion to determine the limit of detection (LOD) of both nanoprobes. FIG. 19 C shows the LOD for IR792 based nanoprobes to be 1.0 fM, while 3-based nanoprobes had a 10-fold lower LOD, 100 aM. To our knowledge this is the lowest reported LOD utilizing a biologically relevant NIR excitation source. We also evaluated the serum stability of the 3-based SERRS-nanoprobe. The SERRS-nanoprobe was shown to be serum stable (e.g. no significant difference between t=1 h and t=48 h) for at least 48 hours. This is supported by a previous study showing that SERS-nanoparticles of similar size and composition remain stable in vivo for more than 2 weeks.

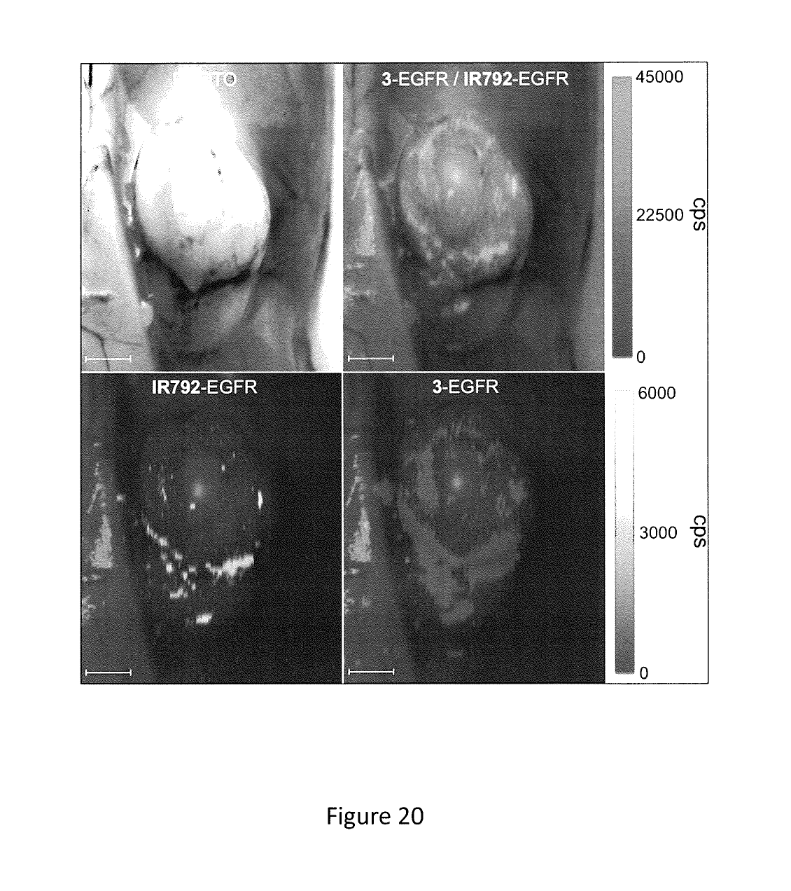

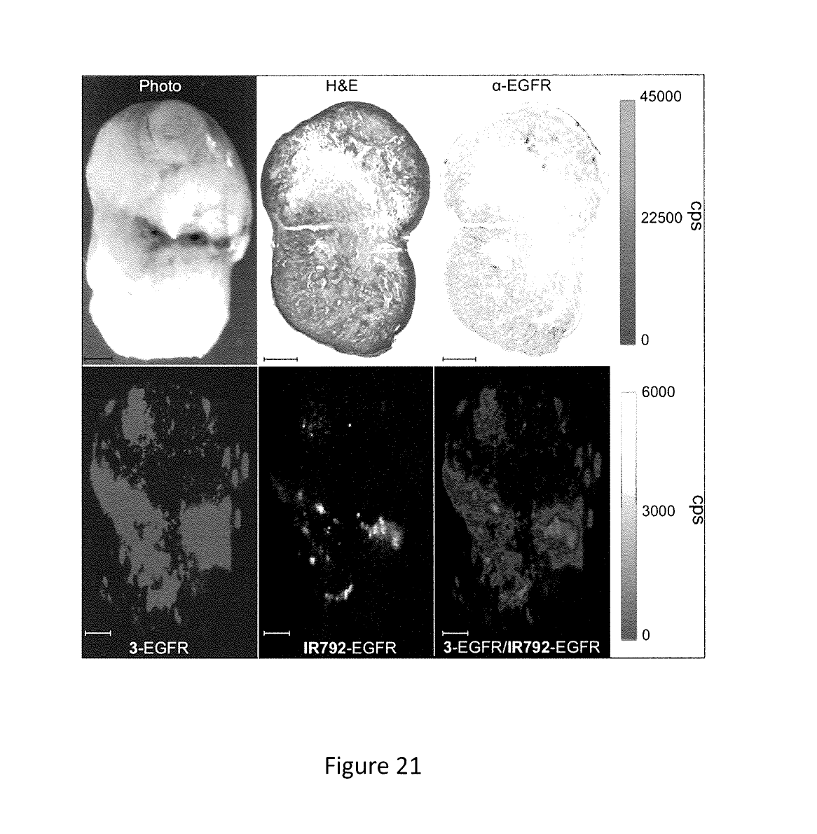

In vivo comparison of EGFR-targeted CP-3- or IR792-SERRS-nanoprobes. The ability of our SERRS-nanoprobe to delineate tumor tissue in vivo was assessed by utilizing CP dye 3 and IR792-based SERRS-nanoprobes functionalized with an epidermal growth factor receptor (EGFR)-targeting antibody. Equimolar amounts (15 fmol/g) of these two EGFR-targeted nanoprobes were injected intravenously into athymic nude mice which had been inoculated two weeks prior with the EGFR-overexpressing cell line A431 (1×10⁶ cells). After 18 hours, the skin around the tumor was carefully peeled back and multiplexed Raman imaging the tumor site and surrounding tissue was performed (FIG. 20-21). A Raman map was generated and the signals from the multiplexed SERRS-Nanoprobes were deconvoluted by applying a direct classical least square algorithm (DCLS). The SERRS-signal from both nanoprobes was more intense for the tumor site than for the surrounding tissue, showing that the EGFR-targeted SERRS-nanoprobes had selectively localized at the tumor site. The SERRS-signal intensity at the tumor mass revealed a 3× higher signal density for the 3-based SERRS-nanoprobes than for the otherwise identical IR792-based SERRS-nanoprobes. Ex vivo multiplexed Raman imaging of the tumor showed Raman signal of the EGFR-targeted SERRS-nanoprobes throughout the tumor with the exception of a hypointense Raman region in the center of the tumor. H&E and immunohistochemical staining for EGFR revealed that the hypointense Raman region corresponded with an area of necrosis, which explains the lack of SERRS-nanoprobe accumulation and decreased Raman signal. In addition, to validate EGFR targeting, we injected A431-tumor bearing mice with cetuximab (50 pmol/g) 3 hours prior to injection with the EGFR-targeted SERRS-nanoprobes. Pre-blocking of EGFR by cetuximab resulted in decreased accumulation of the EGFR-targeted SERRS-nanoprobes within the tumors of animals that were injected with cetuximab prior to EGFR-targeted SERRS-nanoprobe injection as compared to animals that were injected with EGFR-targeted SERRS-nanoprobes and were not pre-injected with cetuximab.

Discussion Effective biomedical imaging requires low limits of detection and high specificity for biological targets. Raman imaging has surfaced as an optical imaging modality that has the promise to enable both. While the Raman effect is relatively weak (1 in 10⁷ photons), the Raman scattering cross section of a molecule can be massively amplified by noble metal surfaces. Here, we demonstrated that rational SERRS-reporter design afforded SERRS-nanoprobes with unprecedented limits of detection: 100 attomolar. This is to the best of our knowledge the lowest reported limit of detection at near-real-time detection (≦2.0 s acquisition times) for SERRS-nanoprobes that are compatible with a NIR light source. As a comparison non-resonant SERS-nanoprobes are in the 0.1-1.0 pM range (1,000-10,000-fold less sensitive), while reported detection limits of SERRS-nanoprobes are >17 fM at near real-time detection. Others have reported a 0.4 fM detection limit, however, this was acquired through cumulative data acquisition with an acquisition time ≧60 s, which is not practical for biomedical imaging applications.

We believe the unprecedented limit of detection of our novel SERRS-nanoprobe is due to several factors. First, we demonstrate that rational design and optimization of the SERRS-reporter is important to achieve efficient “loading” on the nanoparticle. Our results demonstrate that the counterion and gold surface affinity are important considerations. For instance, while the chaotropic PF₆⁻ anions stabilized the dye-nanoparticle system during silica shell formation in ethanol, the system becomes more destabilized with Cl⁻ (more kosmotropic) ions present. Chloride-induced aggregation of colloidal dispersions in relation to SERS has been studied. Natan et al. demonstrated that the strongest enhancements were obtained from aggregates with effective diameters of less than 200 nm and aggregates with sizes >200 nm did not generate appreciable SERS intensities. The aggregates that were induced by the chloride counterion in our system were >200 nm, which might explain the reduced SERRS-signal when chloride is used as a counterion. Others have shown that the kosmotropic chloride-ion could induce reorientation of the dye on the surface, which could also contribute to the reduced SERRS intensities. However, while we did observe a decrease in the SERRS-signal intensity when chloride is present, we did not find any appreciable differences between the Raman spectra of the dyes when different counterions were use, which would have been expected if the molecule had reoriented on the surface. Since the most chaotropic counterion, PF₆⁻, induced the least aggregation and generated robust SERRS-signal intensities, we used PF₆⁻ as a counterion.

Next, we showed that an increase in affinity of the SERRS-reporter for the gold nanoparticle surface via incorporation of 2-thienyl functional groups considerably increased the SERRS-signal without inducing aggregation. Others have reported the functionalization of NIR dyes with thiol or lipoic acid functional groups. In contrast to a 2-thienyl substituent, thiol and lipoic acid functional groups offer no benefit to the optical properties of the dye, and as a tether, do not allow the dye to be as close to the gold surface. Moreover, based on the absorption spectra of reported lipoic-acid modified cyanine dye-gold nanoparticle conjugates, it is clear that lipoic-acid modified dyes promote aggregation.

Finally, the strategy chosen to stabilize the SERRS-nanoprobe is a key factor. Others have reported using either surfactants or thiolated-polymers to stabilize their SERRS-nanoparticles. However, such stabilizing agents compete with the SERRS-reporter for the surface of the nanoparticle, which leads to relatively low SE(R)RS-signal. We achieved very low limits of detection by using a primerless silication procedure in which the silica not only served as a stabilizing agent, but also as a matrix to contain our optimized CP-based SERRS-reporter. Since silica has much lower affinity for the gold than the applied SERRS-reporters, attomolar limits of detection were achieved.

The chalcogenopyrylium dyes represent a new class of SERRS-reporters. Selection of the right combination of chaotropic counterions and increased affinity of the SERRS-reporter for the gold nanoparticle's surface produces stable SERRS-nanoprobes with exceptionally low limits of detection (attomolar range). The low limit of detection (i.e. close to single nanoparticle detection) in combination with the high resolution of Raman imaging, enables highly sensitive and specific, near-real-time tumor delineation and, as a result of the fingerprint like spectra of the different SERRS-nanoprobes, can offer multiplexed disease marker detection in vivo.

Methods. Dye synthesis and characterization. SERRS-nanoprobe synthesis. Gold nanoparticles were synthesized through addition of 7.5 ml 1% (w/v) sodium citrate to 1000 ml boiling 0.25 mM HAuCl₄. The as-synthesized gold nanoparticles were concentrated by centrifugation (10 min, 7500×g, 4° C.) and dialyzed overnight (3.5 kDa MWCO; 5 L 18.2 MΩ·cm). The dialyzed gold nanoparticles (100 μL; 2.0 nM) were added to 1000 μL absolute ethanol in the presence of 30 μL 99.999% tetraethylorthosilicate (Sigma Aldrich), 15 μL 28% (v/v) ammonium hydroxide (Sigma Aldrich) and 1 μL chalcogenopyrylium dye (1-10 mM) in N,N-dimethylformamide. After shaking (375 rpm) for 25 min at ambient conditions in a plastic container, the SERRS-nanoprobes were collected by centrifugation, washed with ethanol, and redispersed in water to yield 2.0 nM SERRS-nanoprobes.

SERRS-nanoprobe characterization. The as-synthesized SERRS-nanoprobes were characterized by transmission electron microscopy (TEM; JEOL 1200ex-II, 80 kV, 150,000× magnification) to study the SERRS-nanoprobe structural morphology. The size and concentration of the SERRS-nanoprobes were determined on a Nanoparticle Tracking Analyzer (NTA; Malvern Instruments, Malvern, UK). Absorption spectra to determine possible nanoparticle aggregation (typically detectable at wavelengths >600 nm) were measured on an M1000Pro spectrophotometer (Tecan Systems Inc. San Jose, Calif.). Finally Raman spectra were acquired on a Renishaw InVIA system equipped with a 785-nm laser (Renishaw Inc, Hoffman Estates, Ill.). All measurements were performed at a laser power of 50 μW/cm² (1.0 s acquisition time, 5× objective).

SERRS-nanoprobe limit of detection. SERRS-nanoprobes were synthesized as described above in the presence of an equimolar (1.0 μM) amount of 3 or IR792. SERRS imaging to determine the limit of detection was performed at 100 mW/cm² (2.0 s acquisition time (StreamLime™), 5× objective) on a phantom that consisted of a serial diluted IR792- or chalcogenopyrylium dye (3)-based SERRS-nanoprobe redispersed in 10 μL water (concentration range 3000-0.003 fM; n=3). The Raman maps were generated by WiRE 3.4 software (Renishaw) by applying a direct classical least square (DCLS) algorithm. The Raman image was analyzed with ImageJ software and plotted in GraphPad Prism (GraphPad Software Inc., La Jolla, Calif.).

Serum stability. The SERRS-nanoprobes (2.0 nM) were incubated in triplicate in 50% mouse serum (Abd Serotec, Raleigh, N.C.) at 37° C. At the indicated time points, a Raman spectrum was taken (50 μW/cm²; 1.0 s acquisition time; 5× objective). The intensities of the 1600 cm⁻¹ were plotted in GraphPad Prism (GraphPad Software Inc., La Jolla, Calif.).

Animal studies. All animal experiments were approved by the Institutional Animal Care and Use Committees of Memorial Sloan Kettering Cancer Center.

In vivo comparison of EGFR-targeted CP-3- or IR792-SERRS-nanoprobes. Female athymic nude mice (n=5) were inoculated with the EGFR-overexpressing cell line A431 (1×10⁶ cells). After 2 weeks, the mice were injected with an equimolar amount (15 fmol/g) of EGFR-targeted IR792- and 3-based SERRS-nanoprobes. The EGFR-targeted SERRS-nanoprobes were synthesized as described above in the presence of an equimolar (1.0 μM) amount of 3 or IR792. The as-synthesized SERRS-nanoprobes were subsequently functionalized with sulfhydryl-groups by heating the SERRS-nanoprobes in 5 mL 2% (v/v) mercaptotrimethoxysilane (MPTMS) in ethanol at 70° C. for 2 hours. The sulfhydryl-functionalized SERRS-nanoprobes were washed and conjugated to an EGFR-targeting antibody (cetuximab; Genentech, South San Francisco, Calif.) through a 4000 Da heterobifunctional maleimide/N-hydroxysuccinimide polyethylene glycol linker. Eighteen hours later, the mice were sacrificed by CO₂-asphyxiation. The tumor was exposed and scanned by Raman imaging (10 mW/cm², 1.5 s acquisition time (StreamLime™), 5× objective). The Raman maps were generated by WiRE 3.4 software (Renishaw) by applying a direct classical least square (DCLS) algorithm.

Immunohistochemical staining. The tissues from the imaging studies were collected and fixed in 4% paraformaldehyde, 4° C. overnight and subsequently processed to be embedded in paraffin. The Discovery XT biomarker platform (Ventana, Tucson, Ariz.) was used to stain the tissue sections (5 μm). Heat-induced epitope retrieval was performed using the citrate buffer (pH 6.0). The primary anti-EGFR antibody (D38B1, Cell Signaling Technology, Danvers, Mass.) was diluted 1:150. The biotin-labeled secondary anti-rabbit antibody (BA-1000, Vector Laboratories) was diluted 1:300.

### Example 3

In this example, we describe the design of SERS nanotags that operate with 1280-nm excitation. The nanotags are based on hollow gold nanoshells (HGNs) and reporter molecules selected from a small library of (chalcogenopyranyl)chalcogenopyrylium monomethine (1-8) and trimethine dyes (9-14) substituted with phenyl, 2-thienyl, and 2-selenophenyl substituents at the 2- and 6-positions of the pyrylium/pyranyl rings (Scheme 1 in Example 1). Dye 14 with two sulfur atoms in the thiopyrylium/thiopyranyl core and four 2-selenophenyl substituents at the 2,2′,6,6′-positions was exceptionally bright in this library of reporters. All fourteen members of the reporter library can be uniquely identified by principal component analysis of their SERS spectra.

Results. The syntheses of 1-14 are shown in Scheme 1 and the library was constructed by condensation of 4-methylthiopyrylium and 4-methylselenopyrylium salts 15 either with chalcogenopyranones 16 or with (4-chalcogenopyranylidene)acetaldehyde derivatives 17 in acetic anhydride to give monomethine dyes 1-8 or trimethine dyes 9-14, respectively. 4-Methylthiopyrylium and 4-methylselenopyrylium salts 15 were prepared by the addition of MeMgBr to the corresponding chalcogenopyranone 16 followed by treatment with aqueous HPF₆. Synthetic details are provided in the Supporting Information. Values of absorption maxima, λₘₐₓ, in CH₂Cl₂ for 1-8 varied from 653 nm for 1 to 724 nm for 6 and values of the molar extinction coefficient, ε, were in the range of 1.1×10⁵ to 1.5×10⁵ M⁻¹ cm⁻¹. For trimethine dyes 9-14, values of λₘₐₓ in CH₂Cl₂ varied from 784 nm for dye 10 to 826 nm for dye 14 while values of E were in the range of 2.0×10⁵ to 2.8×10⁵ M⁻¹ cm⁻¹. The interchange of S and Se atoms in the chalcogenopyrylium backbone, the use of monomethine and trimethine bridges, and the interchange of phenyl, 2-thienyl, and 2-selenophenyl substituents at the 2-,2′-, 6-, and 6′-positions allow the fine tuning of wavelengths of absorption and allow each dye to have a unique Raman fingerprint.

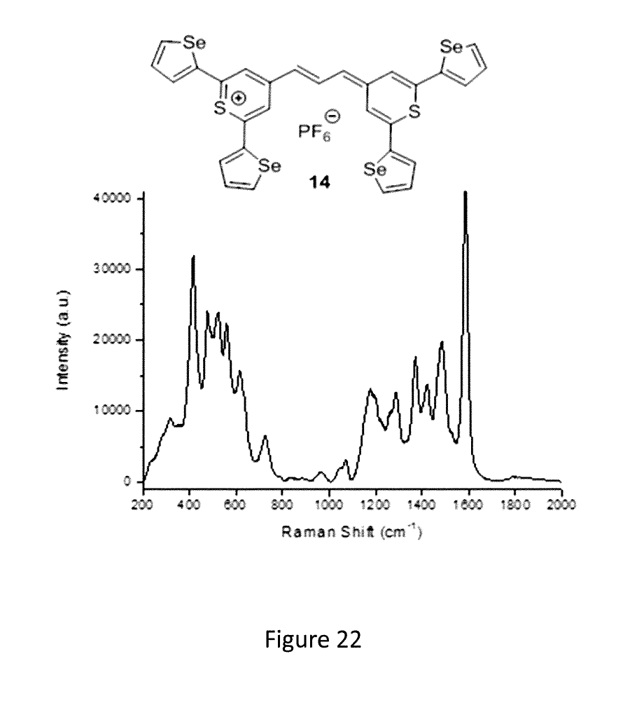

Raman scattering tends to be weak in the near infrared (NIR) region due to its dependence on the 4th power of the excitation frequency. However, the scattering effect can be significantly enhanced by trapping molecules close to the roughened surface of metallic nano-substrates and in this case, the gold surface of HGNs. The enhancement obtained from SERS is related to the frequency of the surface plasmon excited on the metal rather than the 4th power law. Therefore, to make SERS a viable method in the NIR region, and specifically at 1280 nm where no SERS nanotags have previously been reported to be compatible, the surface plasmon resonance (SPR) must be resonant with the NIR excitation source. We engineered the combination of HGNs and dyes 1-14 as SERS nanotags to produce SERS signals with 1280-nm excitation. The SERS spectrum of dye 14 is shown in FIG. 22.

There are three important components which make up these '1280 SERS nanotags, the first being the SERS substrate. For this study we have chosen HGNs as these nanostructures have strong SERS properties. Also, HGNs have desirable characteristics such as small size (usually from 50-80 nm), spherical shape and a strong tunable plasmon band from the visible to the NIR region. Commonly, Ag and Au spherical nanoparticles that have plasmon bands in the visible region are used as SERS substrates. However, these nanoparticles in conjunction with dyes 1-14 produced much weaker SERS signals than the HGNs due to their lack of red-shifted SPR.

The second necessary component of SERS nanotags is the Raman reporter. The thiophene and selenophene-substituted chalcogenopyrylium dyes were specifically designed as Raman reporters for use in the NIR region. Since the SERS effect decreases exponentially as a function of distance from the nanoparticle, it is important that the Raman reporter be near the Au surface. The dyes 1-14 incorporate S and Se atoms in the chalcogenopyrylium core to provide attachment to Au and the 2-thienyl and 2-selenophenyl groups on select members of this library provide novel attachment points to Au for Raman reporters. Earlier studies have shown that thiophenes and selenophenes are both capable of forming self-assembled monolayers on Au. Selenolates have also been shown to have greater affinity for Au than thiolates.

It can be seen in FIG. 22 that these dyes are highly aromatic and produce vibrationally rich and intense SERS spectra with a laser excitation of 1280 nm. The trimethine dyes 9-14 produce more intense signals than their monomethine counterparts (dyes 1-8), with the selenophene-substituted reporters producing stronger SERS spectra than the thiophene-substituted dyes. The SERS spectra for dyes 1-13 were acquired with a 7-s acquisition time with the 1280-nm laser. The SERS spectrum of dye 14 with four 2-selenophenyl substituents was collected with only 3-s acquisition time due to the signal intensity saturating the spectrometer. Dye 13 with four 2-thienyl substituents gave a weaker SERS signal compared to dye 14 with four 2-selenophenyl substituents. This suggests that the selenophene group adheres more effectively to the gold surface than thiophene and supports previous reports where selenolates have shown a greater affinity for gold surfaces than thiolates.

Both dye 13 and dye 14 are significantly red-shifted with light absorption maxima >800 nm, making them NIR active. Another benefit of these dyes is the multiple S and Se atoms incorporated into their structures allowing them to adsorb onto the HGN surface very strongly and experience a larger enhancement.

X-ray structural studies have shown that the chalcogenopyrylium/chalcogenopyranyl rings and the methine carbon of chalcogenopyrylium monomethine dyes related to 1-8 are coplanar and computational studies predict similar coplanarity in chalcogenopyrylium trimethine dyes 9-14. Other studies have shown that a 2-thienyl group can be coplanar with an attached thiopyranyl ring. X-ray crystallographic analysis of single crystals of dye 14 indicate that the thiopyrylium/thiopyranyl trimethine core and the four 2-selenophenyl substituents are coplanar as shown in FIG. 1. In essence, all six chalcogen atoms can be involved in binding the reporter to the Au surface. Furthermore, the 2-selenophenyl substituents can rotate from coplanar with the thiopyrylium/thiopyranyl trimethine core to any angle to give the strongest binding to the HGN surface.

The third component in the SERS nanotag is the aggregating agent, usually a simple inorganic salt such as potassium chloride (KCl) that screens the Coloumbic repulsion energy between the nanoparticles, allowing the reporter molecules to adhere more closely to the nanoparticle surface. Although the aggregating agent was necessary for most of the dyes, it is important to note that with chalcogenopyrylium dyes 13 and 14, KCl was not required for intense signals to be observed. This is possibly due to a strong interaction occurring between the reporter and HGN surface inducing self-aggregation. This partial aggregation observed from these nanotags perhaps widens the scope for future SERS applications where aggregating agents are not required and the aggregation of the nanoparticles comes solely from a biological recognition event such as DNA-DNA interactions, DNA-protein interactions, peptide-protein interactions or sugar-protein interactions. Furthermore, these nanotags could be used as alternative reporters in biological applications such as photothermal ablation therapy or optical coherence tomography where there is a great need for NIR active materials.

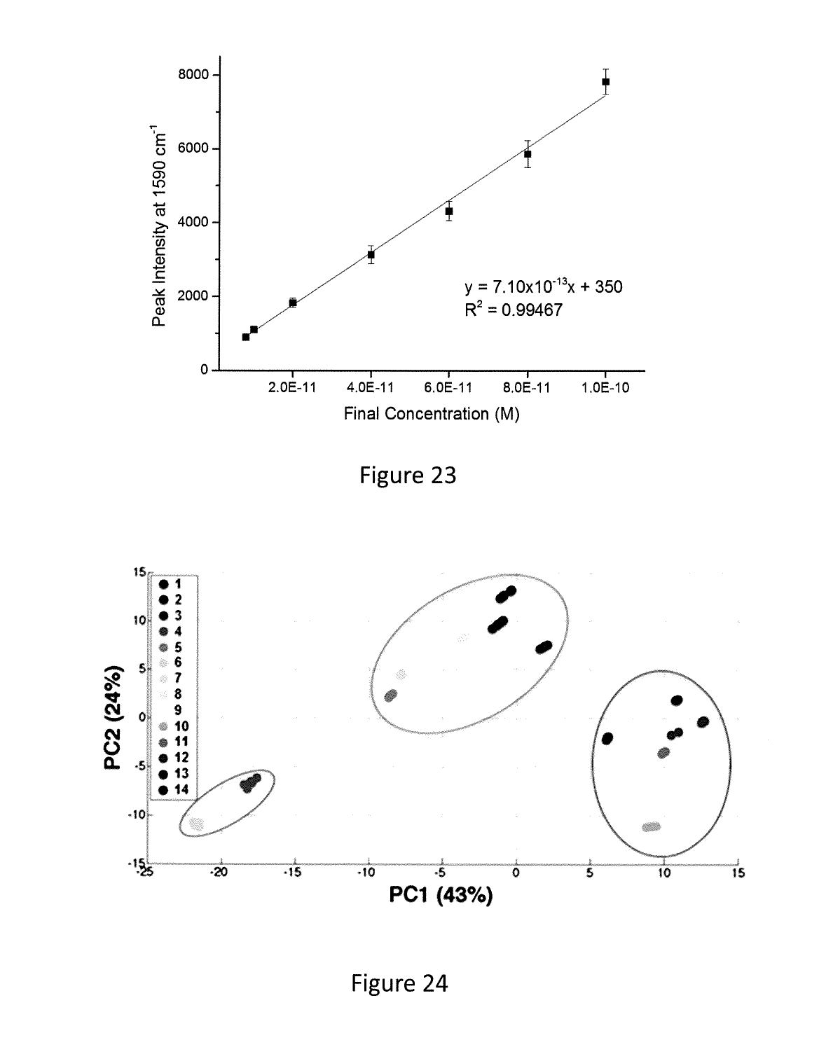

Due to the exceptional response obtained with dye 14 and HGNs, particle dilution studies were conducted in order to calculate a limit of detection (LOD) for this dye at this extremely red-shifted laser wavelength. The LOD study was carried out by initially using the optimum conditions (those used in FIG. 22 to obtain SERS at 1280 nm and detailed in the Supporting Information) in which the dye concentration was 1.93 nM and then subsequent dilutions in water were made until no signal from dye 14 was observed. The peak at 1590 cm⁻¹, which should arise from heterocyclic aromatic ring stretching within the molecule, was used to calculate the LOD since it was the most intense peak in the spectrum. FIG. 23 shows that a linear response was followed and a LOD of 1.47 pM was calculated. The limit of detection was calculated to be 3 times the standard deviation of the blank, divided by the gradient of the straight line in FIG. 23.

In addition, the non-resonant commercial dyes BPE (1,2-bis(4-pyridyl)ethylene) and AZPY (4,4-azopyridine), which are commonly used with Au nanosubstrates for SERS analysis were also tested with the HGNs at this laser wavelength but failed to produce a SERS signal. Until now, no SERS nanotags compatible with the critical laser excitation wavelength of 1280 nm have been reported. We have demonstrated a range of nanotags that show excellent SERS properties and a LOD in the picomolar range. This work provides the basis for advancement in the SERS field with these nanotags showing promise for future use in a wide range of optical applications.

Furthermore, these dyes can be separated out and individually identified in a reproducible manner based on their unique structures and SERS spectra by performing multivariate analysis in the form of principal component analysis (PCA). PCA is employed to reduce the dimensionality of the spectroscopic data while making it easier to identify variations in the SERS spectra. PCA was carried out on 14 data sets consisting of the spectra obtained from each individual dye experiment. The resulting principal component (PC) scores plot (FIG. 24) clearly illustrates three unique groupings. The red cluster contains the trimethine dyes 9-14; these reporter molecules produced the most intense SERS signals and all contain 3 sp² carbons in their structural backbone. The blue clustering highlights the monomethine dyes (1-3,5,7-8) which are good Raman reporters and produce intense SERS spectra with HGNs and KCl while the green cluster contains the two dyes which didn't produce any SERS response (dyes 4 and 6) when excited with the 1280 nm laser. The monomethine dyes only contain 1 sp² carbon in their backbone and this simple difference in molecular structure could be responsible for the variation in signal intensities observed between the trimethine and monomethine dyes. Moreover, this simple structural change can affect the distance, orientation and/or the polarizability of the reporter which ultimately affects the SERS response. Additionally it can be observed that within the three groupings all 14 dyes can be individually identified by PCA and classified according to their unique structure and SERS spectra. The replicates for each dye are tightly clustered illustrating the excellent reproducibility of the SERS spectra. Therefore, a further benefit for these 1280 SERS nanotags is that they could be employed in future multiplexing systems, where multiple analytes need to be identified simultaneously, such as in chemical or medical detection assays.

A new extreme red shifted SERS nanotag was designed and synthesized to demonstrate unprecedented performance using 1280 nm excitation. This was achieved by combining a set of chalcogen dyes with hollow gold nanoshells to provide a unique performance at this longer wavelength of excitation. These dyes with the more widely used Au nanoparticles or HGNs with conventional Raman reporters such as BPE were unable to match the combined performance of the chalcogenopyrylium dyes and HGNs indicating the unexpected and superior performance of SERS nanotags based on the combination of these dyes and the tunable HGNs. This significant result now makes SERS nanotags available for use at wavelengths suitable for deep tissue analysis.

The preceding description provides specific examples of the present disclosure. Those skilled in the art will recognize that routine modifications to these embodiments can be made which are intended to be within the spirit and scope of the disclosure.

1. A compound having the following structure: 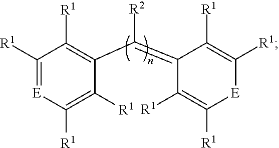
wherein,
each E is, at each occurrence in the compound, independently charged or neutral and is independently selected from S, Se, O, or Te, wherein at least one E is S or Se;
each R¹ is, at each occurrence in the compound, independently selected from the group consisting of —H, C₁₋₈ alkyl group, halo group, —CN, aryl group, and heteroaryl group and adjacent R¹ groups can combine to form C₅₋₈ aryl groups;
each R², at each occurrence in the compound, is independently selected from the group consisting of —H, C₁₋₈ alkyl group, halo group, —CN, and aryl group, R² groups beta to each other can combine to form C₅₋₈ cycloalkyl groups, C₅₋₈ aryl groups or C₅₋₈ heteroaryl groups, and n is an odd number from 1 to 7; and
Z is optionally present and is a counter ion.

2. The compound of claim 1, wherein the compound has one of the following structures:

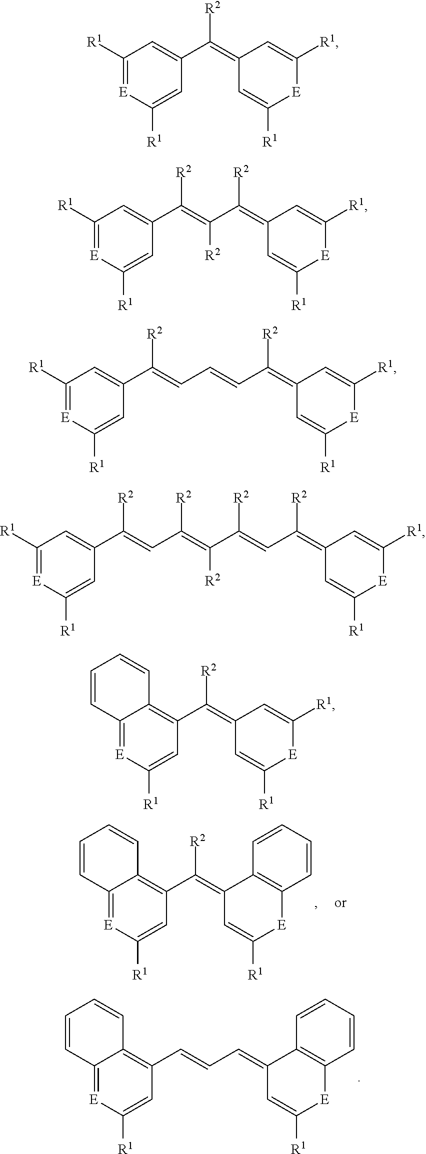

3. The compound of claim 1, wherein the compound has one of the following structures:

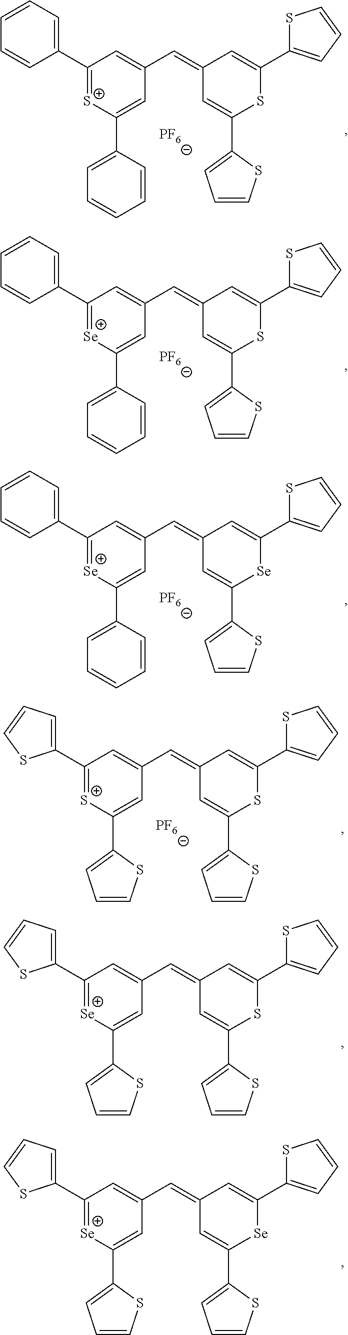

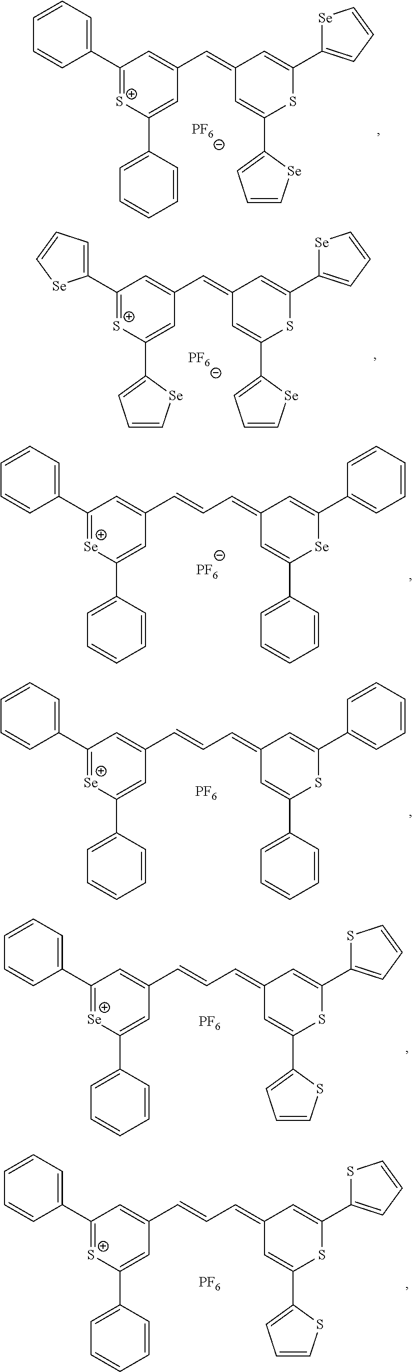

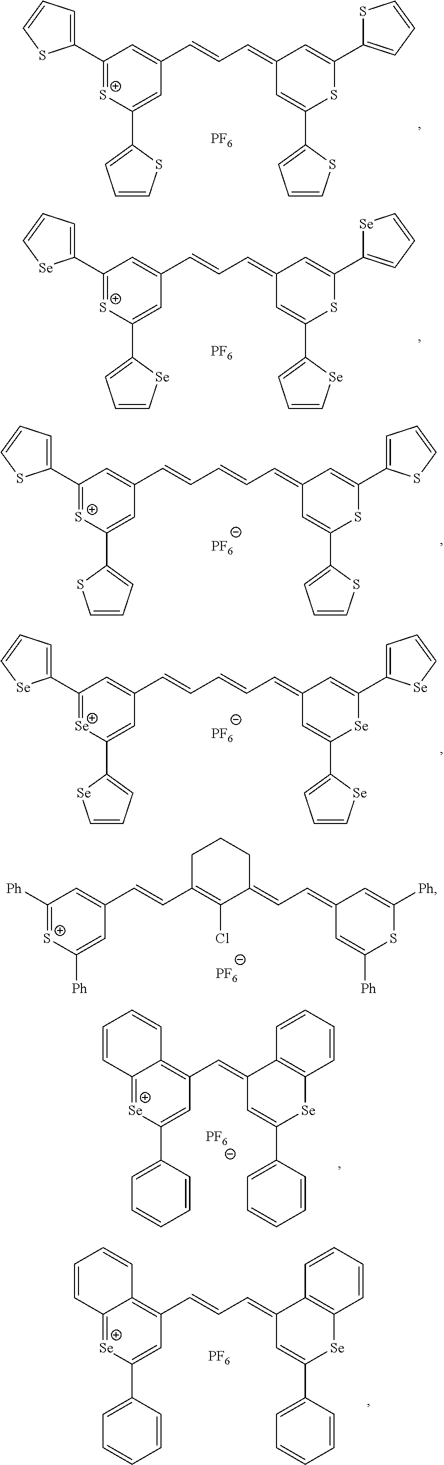

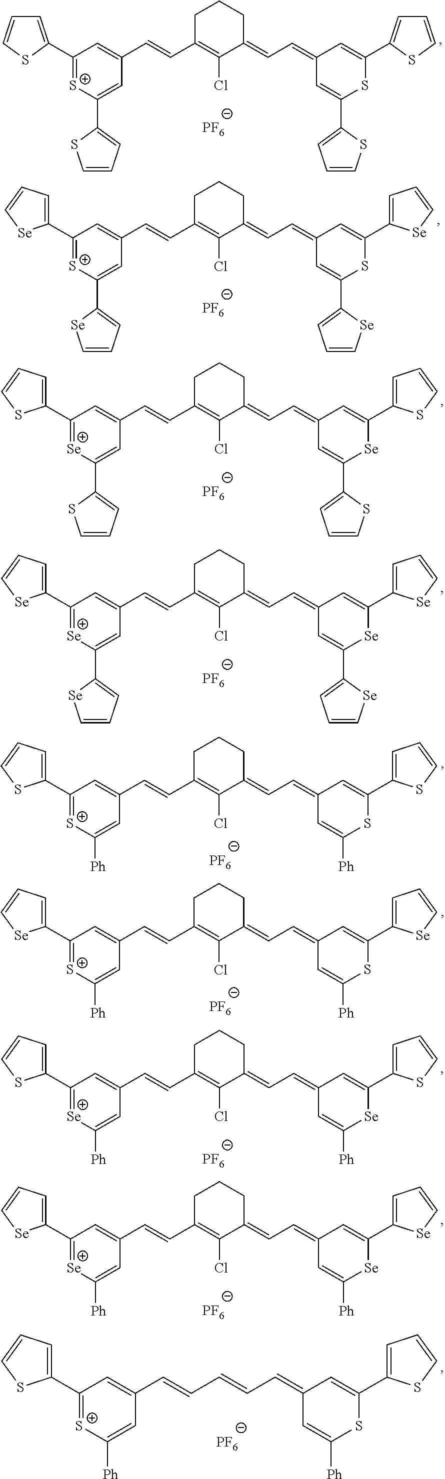

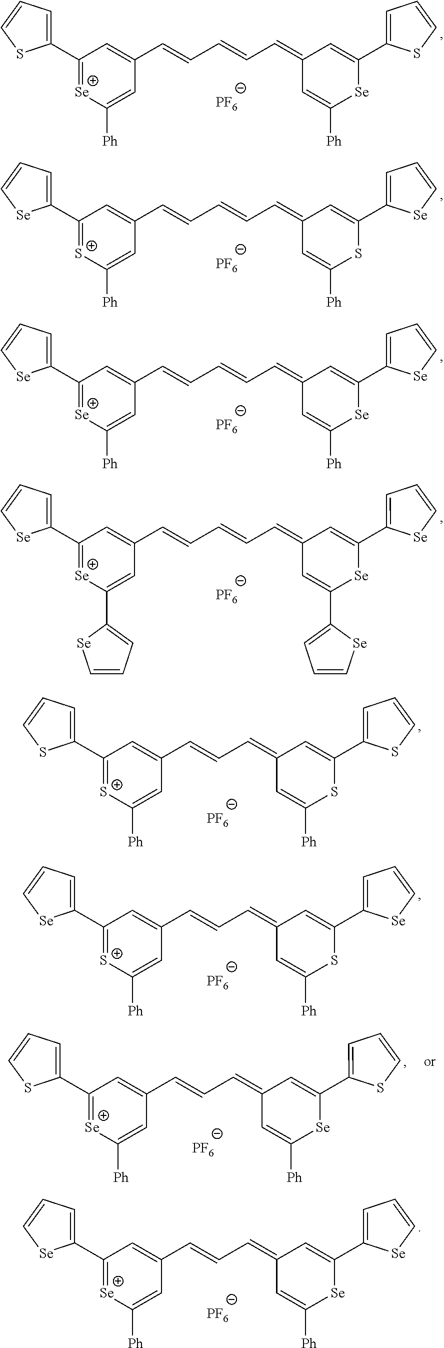

4. A composite nanostructure comprising:
a core comprising a nanomaterial;
one or more reporter molecules having the structure of claim 1, wherein each of the reporter molecules is independently, at each occurrence in the composite nanostructure, directly covalently bound to the core or covalently bound to the core via a linking group to the core; and
optionally, an encapsulating material that at least partially encapsulates the core and the one or more reporter molecules.

5. The composite nanostructure of claim 4, wherein the core comprises a metal nanomaterial.

6. The composite nanostructure of claim 4, wherein the core is a hollow gold nanoshell.

7. The composite nanostructure of claim 4, wherein the nanomaterial is a nanoparticle and the nanoparticle size is 15 to 300 nm.

8. The composite nanostructure of claim 4, wherein the nanostructure morphology is selected from the group consisting of sphere, rod, star, raspberry, and hollow shell.

9. The composite nanostructure of claim 4, wherein the encapsulating material is an inorganic material, polyethylene glycol (PEG), or organic polymer.

10. The composite nanostructure of claim 4, further comprising one or more targeting moieties directly covalently bound to the core or covalently bound to the core via a linking group,
wherein the encapsulating material, if present, at least partially encapsulates the core, the one or more reporter molecules, and the one or more targeting moieties, if present, are directly bound or covalently bound via a linking group to an outer surface of the encapsulating material.

11. A method of making a composite nanostructure of claim 4, comprising binding one or more reporter molecules of the present invention to a core, and optionally, encapsulating the core and reporter molecule within an encapsulating material.

12. A method for detecting one or more target molecules in a sample comprising:
contacting an individual with one or more composite nanostructures of claim 10,
obtaining surface-enhanced Raman spectroscopy data of a portion of the individual after contact of the portion of the individual with the one or more said composite nanostructures, wherein observation of surface-enhanced Raman spectroscopy data attributable to a particular composite nanostructure of the one or more said composite nanostructures indicates the presence of the target molecule in the portion of the individual corresponding to the targeting moiety of the particular nanostructure.

13. The method of claim 12, further comprising obtaining surface-enhanced Raman spectroscopy data of one or more additional portions of the individual after contact of the one or more additional portions of the individual with the one or more said composite nanostructures.

14. The method of claim 12, further comprising generating an image of at least a portion of the individual using the surface-enhanced Raman spectroscopy data from the portion and, optionally, additional portions of the individual.
